(19) United States
(12) Patent Application Publication
(10) Pub. No.: US 2024/0423210 A1
(43) Pub. Date: Dec. 26, 2024

(54) PREDATORY MITES WITH IMPROVED TRAITS

(71) Applicant: BIO-BEE SDE ELIYAHU LTD., Emmek, Hama’yanot (IL)

(72) Inventors: Arnon TABIC, Bet Keshet (IL); Reut BEN AMI, Kibbutz Neve Ur (IL); Shimon STEINBERG, Timrat (IL)

(73) Assignee: BIO-BEE SDE ELIYAHU LTD., Emmek, Hama’yanot (IL)

(21) Appl. No.: 18/689,937

(22) PCT Filed: Sep. 7, 2022
(86) PCT No.: PCT/IL2022/050974

§ 371 Date: Mar. 7, 2024

Related U.S. Application Data
(60) Provisional application No. 63/260,955, filed on Sep. 7, 2021.

(51) Int. Cl. A01N 63/16; A01K 67/033; A01P 7/02; C12Q 1/6888

(52) U.S. Cl. CPC A01N 63/16; A01K 67/033; A01P 7/02; C12Q 1/6888; A01K 2227/70; C12Q 2600/156

(57) ABSTRACT

The present invention discloses a biological control composition comprising a cultivated rearing population of Phytoseiulus persimilis (P. persimilis) predatory mite individuals, and optionally a carrier material, wherein at least 5% of the P. persimilis individuals comprise within their genome at least one Single Nucleotide Polymorphism (SNP) marker associated with the trait of improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

### FIELD OF THE INVENTION

The present invention relates to the field of biological control agents for crop protection, and more particularly to novel means and methods for rearing biological control agents against plant pests.

### BACKGROUND OF THE INVENTION

The use of arthropods (insects and mites) as Biological Control Agents (BCA) is an expanding field with many advantages over chemical pest control. Arthropod BCA's are able to naturally control other arthropod species that act as pests on the crop.

Phytoseiulus is a genus of predatory mites in the family Phytoseiidae. Mite species of this genus are the most frequently used BCA to control two-spotted spider mites in greenhouses and outdoor crops grown in mild environments. A Phytoseiulus mite can consume up to seven adult spider mites or several dozens of their eggs in a day. A well-fed female lays about 50 eggs in her lifetime. The genus Phytoseiulus contains five known species, namely: P. persimilis, P. longipes, P. macropilis, P. fragariae and P. robertsi. All species of the genus Phytoseiulus are considered Type I Specialized Predators of Tetranychus Species, i.e. highly specific to a diet consisting of spider mites, preferably of the genus Tetranychus (McMurtry and Croft 1997). This is further strengthened by the publication of McMurtry et al. (2013), supporting that Phytoseiulus species (Type Ta predators) only feed and reproduce on their natural prey. This publication recites “Subtype I-a—Specialized predators of Tetranychus (Tetranychidae)”. This category is considered to be genus-specific with regard to prey preference and is represented only by Phytoseiulus species. The most frequently used species of this genus for biological control of spider mites is Phytoseiulus persimilis.

Phytoseiulus persimilis Athias-Henriot (Acari: Phytoseiidae) is a specialist predator on Tetranychid mites, especially on the two-spotted spider mite, Tetranychus urticae Koch (Acari: Tetranychidae).

From a commercial point of view, a significant disadvantage of mass producing a predatory mite that exclusively feeds on phytophagous mites, such as spider mites, is that it requires rearing prey mites on plants, which involves high rearing costs and complex operations (such as providing appropriate plants in sufficient abundance, under greenhouse conditions).

WO2019171374 and WO2021044404 publications report for the first time successful rearing of Phytoseiulus mite species on a non-Tetranychid arthropod prey.

However, although predatory mites (Acari: Phytoseiidae) are the most important beneficial arthropods used in biological pest control of protected crops around the world, the genomes of mites are far less understood than, for example, those of insects.

In view of the above, there is a long felt and unmet need for generation of predatory Phytoseiidae mites with improved genetic traits.

### SUMMARY OF THE INVENTION

The present invention relates to the field of insect control and more specifically to a system and method for rearing biological control agents against plant pests.

It is one object of the present invention to disclose a biological control composition comprising a cultivated rearing population of Phytoseiulus persimilis (P. persimilis) predatory mite individuals, and optionally a carrier material, wherein at least 5% of said P. persimilis individuals comprise within their genome at least one Single Nucleotide Polymorphism (SNP) marker selected from: (a) SNP at position 34739 of SEQ ID NO:84 or a variant, portion or complementary sequence thereof, (b) at least one SNP at a position selected from 105452, 109775, 115020, 115073, 125940, 131548 and 131555, of SEQ ID NO:85 or a variant, portion or complementary sequence thereof, (c) at least one SNP at a position selected from 99333, 99595, 99605, 115325 and 115354, of SEQ ID NO:86 or a variant, portion or complementary sequence thereof, (d) at least one SNP at a position selected from 4508, 11697, 12229, 13135, 32979, 38081, 38220, of SEQ ID NO:87 or a variant, portion or complementary sequence thereof, (e) at least one SNP at a position selected from 30913, 34950, 38409, 38458, 42427, 42604 and 43158 of SEQ ID NO:88 or a variant, portion or complementary sequence thereof, (f) SNP at position 1032 of SEQ ID NO:89 or a variant, portion or complementary sequence thereof, (g) SNP at position 173977 and/or SNP at position 228195 of SEQ ID NO:90 or a variant, portion or complementary sequence thereof, and (h) at least one SNP at a position selected from 857, 15689, 40135 and 49492 of SEQ ID NO:91 or a variant, portion or complementary sequence thereof.

It is a further object of the present invention to disclose the biological control composition as defined above, wherein the at least one SNP is selected from: (a) SNP of G at position 34739 of SEQ ID NO:84 or a variant, portion or complementary sequence thereof, (b) at least one SNP selected from C at position 105452, G at position 109775, A at position 115020, T at position 115073, C at position 125940, G at position 131548, G at position 131555, of SEQ ID NO:85 or a variant, portion or complementary sequence thereof, (c) at least one SNP selected from A at position 99333, G at position 99595, T at position 99605, A at position 115325, C at position 115354, of SEQ ID NO:86 or a variant, portion or complementary sequence thereof, (d) at least one SNP selected from A at position 4508, A at position 11697, C at position 12229, A at position 13135, C at position 32979, G at position 38081, C at position 38220 of SEQ ID NO:87 or a variant, portion or complementary sequence thereof, (e) at least one SNP selected from A at position 30913, C at position 34950, C at position 38409, A at position 38458, T at position 42427, G at position 42604, T at position 43158 of SEQ ID NO:88 or a variant, portion or complementary sequence thereof, (f) SNP of G at position 1032 of SEQ ID NO:89 or a variant, portion or complementary sequence thereof, (g) SNP of A at position 173977 and/or SNP of T at position 228195 of SEQ ID NO:90 or a variant, portion or complementary sequence thereof, and (h) at least one SNP selected from C at position 857, T at position 15689, C at position 40135, G at position 49492 of SEQ ID NO:91 or a variant, portion or complementary sequence thereof.

It is a further object of the present invention to disclose the biological control composition as defined in any above, wherein said variant comprises at least 90% sequence similarity to the corresponding SEQ ID nucleotide sequence.

It is a further object of the present invention to disclose the biological control composition as defined in any of the above, wherein at least 5.5%, such as at least 10%, 20%, 30%, 40%, 50%, 60%, 70%, 80%, at least 90% or 100% of said P. persimilis predatory individuals of said cultivated population comprise said at least one SNP marker.

It is a further object of the present invention to disclose the biological control composition as defined in any of the above, wherein the at least 5% of said P. persimilis predatory individuals comprise within their genome a polynucleotide genomic sequence selected from SEQ ID NO:1-28 and SEQ ID NO:84-91, and any combination thereof.

It is a further object of the present invention to disclose the biological control composition as defined in any of the above, wherein the at least 5% of said P. persimilis predatory individuals comprise within their genome a polynucleotide genomic sequence selected from SEQ ID NO:1 to SEQ ID NO:91, and any combination thereof.

It is a further object of the present invention to disclose the biological control composition as defined in any of the above, wherein the cultivated rearing population of P. persimilis predatory mite individuals is characterized by improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

It is a further object of the present invention to disclose the biological control composition as defined in any of the above, wherein the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

It is a further object of the present invention to disclose the biological control composition as defined in any of the above, wherein the composition further comprises as a food source for the P. persimilis predatory mites of the cultivated rearing population, a non-Tetranychid arthropod prey, preferably an immobilized non-Tetranychid arthropod prey, such as on an Astigmatid prey, most preferably on an immobilized Astigmatid prey.

It is a further object of the present invention to disclose the biological control composition as defined in any of the above, wherein the non-Tertranychid arthropod prey is selected from an Astigmatid mite species, an Artemia species, species from the Phytoseiidae family, Thrips such as Frankliniella occidentalis, Tarsonemidae mite species, and lepidopteran eggs such as of Ephestia kunhiela.

It is a further object of the present invention to disclose the biological control composition as defined in any of the above, wherein said Astigmata prey species is selected from members of the family Acaridae, Carpoglyphidae, Glycyphagidae, and/or from the family Chortoglyphagidae.

It is a further object of the present invention to disclose the biological control composition as defined in any of the above, wherein the at least one species from the order Astigmata is selected from members of a genus such as Acarus, Tyrophagus, Aleuroglyphus, Lardoglyphus, Caloglyphus, Suidasia, Thyreophagus, Carpoglyphus, Glycyphagus, Lepidoglyphus, Blomia, and/or Chortoglyphus.

It is a further object of the present invention to disclose the biological control composition as defined in any of the above, wherein the at least one species from the order Astigmata is selected from: Acarus siro, Acarus farris, Acarus immobilis, Acarus chaetoxysilos, Tyrophagus longior, Tyrophagus similis, Tyrophagus putrescentiae, Tyrophagus communis, Aleuroglyphus ovatus, Lardoglyphus konoi, Caloglyphus mycophagus, Suidasia nesbitti, Thyreophagus entomophagus, Carpoglyphus lactis, Carpoglyphus munroi, Glycyphagus domesticus, Lepidoglyphus destructor, Blomia freeman, Blomia tropicalis and/or Chortoglyphus arcuatus.

It is a further object of the present invention to disclose the biological control composition as defined in any of the above, wherein said Astigmatid mite species is selected from:
i) Carpoglyphidae such as from the genus Carpoglyphus e.g. Carpoglyphus lactis;
ii) Pyroglyphidae such as from the genus Dermatophagoides e.g. Dermatophagoides pteronysinus, Dermatophagoides farinae; from the genus Euroglyphus e.g. Euroglyphus longior, Euroglyphus maynei; from the genus Pyroglyphus e.g. Pyroglyphus africanus;
iii) Glycyphagidae such as from the subfamily Ctenoglyphinae, such as from the genus Diamesoglyphus e.g. Diamesoglyphus intermedius, or from the genus Ctenoglyphus, e.g. Ctenoglyphus plumiger, Ctenoglyphus canestrinii, Ctenoglyphus palmifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glycyphagus domesticus, or from the genus Lepidoglyphus e.g. Lepidoglyphus michaeli, Lepidoglyphus fustifer, Lepidoglyphus destructor, or from the genus Austroglycyphagus, e.g. Austroglycyphagus geniculatus; from the subfamily Airoglyphinae, such as from the genus Airoglyphus, e.g. Airoglyphus robustus; from the subfamily Labidophorinae, such as from the genus Gohieria, e.g. Gohieria. fusca; or from the subfamily Nycteriglyphinae such as from the genus Coproglyphus, e.g. Coproglyphus stammeri or from the subfamily Chortoglyphidae, such as the genus Chortoglyphus e.g. Chortoglyphus arcuatus;
iv) Acaridae such as from the genus Tyrophagus e.g. Tyrophagus putrescentiae, Tyrophagus tropicus, Tyrophagus communis from the genus Acarus e.g. Acarus siro, Acarus farris, Acarus gracilis; from the genus Lardoglyphus e.g. Lardoglyphus konoi, from the genus Thyreophagus, such as Thyreophagus entomophagus; from the genus Aleuroglyphus, e.g. Aleuroglyphus ovatus;
v) Suidasiidae such as from the genus Suidasia, such as Suidasia nesbiti, Suidasia pontifica or Suidasia medanensis.

It is a further object of the present invention to disclose the biological control composition as defined in any of the above, wherein the non-Tetranychid arthropod prey comprises immobilized life stages comprising immobilized eggs.

It is a further object of the present invention to disclose the biological control composition as defined in any of the above, wherein said immobilization is carried out by a treatment selected from: thermal treatment, such as freezing, freeze-drying, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof.

It is a further object of the present invention to disclose the biological control composition as defined in any of the above, wherein the improved rearing properties of the cultivated rearing P. persimilis population on a non-Tetranychid arthropod prey, are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals characterized by less than 5% individuals comprising within their genome said at least one SNP marker.

It is a further object of the present invention to disclose the biological control composition as defined in any of the above, wherein said carrier material is selected from particles or elements of sawdust, vermiculite, bran, wheat bran, buckwheat husks, rice husks or millet husks, or comprising a mixture thereof, or any other inert carrier material and/or any carrier having carrier elements comprising surfaces to which mites can adhere or carrier elements comprising mite shelters.

It is a further object of the present invention to disclose the biological control composition as defined in any of the above, wherein said composition is contained in a container such as a sachet, a bottle or any other packaging type container or device configured to holding the biological control composition.

It is a further object of the present invention to disclose a method for obtaining a biological control composition comprising a cultivated rearing population of Phytoseiulus persimilis (P. persimilis) predatory mite individuals as defined in any of the above, wherein said method comprises steps of: (a) providing a rearing population of a P. persimilis predatory mite species, the rearing population comprising individuals of the P. persimilis species, reared on a suitable food source for the Phytoseiulus individuals, said food source comprising a prey species selected from the Tetranychidae species; (b) providing a population of individuals of preselected non-Tetranychid arthropod species, preferably an Astigmatid mite species, most preferably an immobilized Astigmatid mite species having immobilized life stages comprising immobilized eggs; (c) rearing the P. persimilis individuals on the preselected non-Tetranychid arthropod species as a food source; (d) screening the genome of a sample of P. persimilis individuals for the presence of at least 5% of at least one SNP selected from: (i) SNP of G at position 34739 of SEQ ID NO:84 or a variant, portion or complementary sequence thereof, (ii) at least one SNP selected from C at position 105452, G at position 109775, A at position 115020, T at position 115073, C at position 125940, G at position 131548, G at position 131555, of SEQ ID NO:85 or a variant, portion or complementary sequence thereof, (iii) at least one SNP selected from A at position 99333, G at position 99595, T at position 99605, A at position 115325, C at position 115354, of SEQ ID NO:86 or a variant, portion or complementary sequence thereof, (iv) at least one SNP selected from A at position 4508, A at position 11697, C at position 12229, A at position 13135, C at position 32979, G at position 38081, C at position 38220 of SEQ ID NO:87 or a variant, portion or complementary sequence thereof, (v) at least one SNP selected from A at position 30913, C at position 34950, C at position 38409, A at position 38458, T at position 42427, G at position 42604, T at position 43158 of SEQ ID NO:88 or a variant, portion or complementary sequence thereof, (vi) SNP of G at position 1032 of SEQ ID NO:89 or a variant, portion or complementary sequence thereof, (vii) SNP of A at position 173977 and/or SNP of T at position 228195 of SEQ ID NO:90 or a variant, portion or complementary sequence thereof, and (viii) at least one SNP selected from C at position 857, T at position 15689, C at position 40135, G at position 49492 of SEQ ID NO:91 or a variant, portion or complementary sequence thereof; (e) optionally, alternatingly rearing selected Phytoseiulus individuals in a sequence of:
rearing for at least 2, such as between 5 and 50, generations while using a food source comprising the preselected non-Tetranychid arthropod species;
rearing for at least 2, such as between 5 and 50, generations while using a food source comprising the prey species selected from the Tetranychidae
screening the genome of a sample of P. persimilis individuals for the presence of at least 5% of the at least one SNP.

It is a further object of the present invention to disclose the method as defined above, further comprises steps of: (a) separating eggs and/or juvenile developmental stages such as larvae and/or nymphs from the preselected non-Tetranychid arthropod prey rearing population; (b) mixing the separated eggs and/or juvenile developmental stages such as larvae and/or nymphs with a carrier material, such as a carrier material selected from sawdust, wheat bran, buckwheat husks, rice husks or millet husks, or comprising a mixture thereof; (c) freezing the mixture of step (b); and (d) rearing the predatory mite individuals on the mixture as a food source.

It is a further object of the present invention to disclose the method as defined in any of the above, wherein the provided rearing population is a population composed of a number of sub-populations, wherein said sub-populations are from distinct sources, such as from distinct production populations and/or from natural populations isolated from distinct geographical locations.

It is a further object of the present invention to disclose the method as defined in any of the above, wherein the provided rearing population comprises at least 100 individuals, such as between 200 and 5000 individuals, preferably between 500 and 1500 individuals.

It is a further object of the present invention to disclose the method as defined in any of the above, wherein the sample for genome screening comprises at least 50 individuals, such as between 100 and 300 individuals.

It is a further object of the present invention to disclose use of a biological control composition as defined in any of the above, for controlling a pest in a crop.

It is a further object of the present invention to disclose a method for controlling a pest in a crop comprising providing the crop with a biological control composition as defined in any of the above.

It is a further object of the present invention to disclose a method for screening for or identifying a biological control composition as defined in any of the above, the method comprises steps of: (a) obtaining a sample of said population of individuals, the sample preferably comprise at least 50 individuals; (b) pooling the genome said individuals and screening the pooled genome for the presence of at least 5% of at least one SNP selected from:
i. SNP of G at position 34739 of SEQ ID NO:84 or a variant, portion or complementary sequence thereof,
ii. at least one SNP selected from C at position 105452, G at position 109775, A at position 115020, T at position 115073, C at position 125940, G at position 131548, G at position 131555, of SEQ ID NO:85 or a variant, portion or complementary sequence thereof,
iii. at least one SNP selected from A at position 99333, G at position 99595, T at position 99605, A at position 115325, C at position 115354, of SEQ ID NO:86 or a variant, portion or complementary sequence thereof,
iv. at least one SNP selected from A at position 4508, A at position 11697, C at position 12229, A at position 13135, C at position 32979, G at position 38081, C at position 38220 of SEQ ID NO:87 or a variant, portion or complementary sequence thereof,
v. at least one SNP selected from A at position 30913, C at position 34950, C at position 38409, A at position 38458, T at position 42427, G at position 42604, T at position 43158 of SEQ ID NO:88 or a variant, portion or complementary sequence thereof,
vi. SNP of G at position 1032 of SEQ ID NO:89 or a variant, portion or complementary sequence thereof,
vii. SNP of A at position 173977 and/or SNP of T at position 228195 of SEQ ID NO:90 or a variant, portion or complementary sequence thereof, and
viii. at least one SNP selected from C at position 857, T at position 15689, C at position 40135, G at position 49492 of SEQ ID NO:91 or a variant, portion or complementary sequence thereof.

It is a further object of the present invention to disclose a cultivated Phytoseiulus persimilis (P. persimilis) mite individual, wherein said cultivated Phytoseiulus predatory individual comprises within its genome at least one single nucleotide polymorphism (SNP) marker selected from:
a. SNP1 at position corresponding to position 163 of SEQ ID NO:1 or a variant, portion or complementary sequence thereof,
b. SNP2 at position 181 of SEQ ID NO:2 or a variant, portion or complementary sequence thereof,
c. SNP3 at position 197 of SEQ ID NO:3 or a variant, portion or complementary sequence thereof,
d. SNP4 at position 111 and/or SNP5 at position 165 of SEQ ID NO:4 or a variant, portion or complementary sequence thereof,
e. SNP6 at position 193 of SEQ ID NO:5 or a variant, portion or complementary sequence thereof,
f. SNP7 at position 186 and/or SNP8 at position 193 of SEQ ID NO:6 or a variant, portion or complementary sequence thereof,
g. SNP9 at position 161 of SEQ ID NO:7 or a variant, portion or complementary sequence thereof,
h. SNP10 at position 203 and/or SNP11 at position 213 of SEQ ID NO:8 or a variant, portion or complementary sequence thereof,
i. SNP12 at position 163 and/or SNP13 at position 192 of SEQ ID NO:9 or a variant, portion or complementary sequence thereof,
j. SNP14 at position 208 of SEQ ID NO:10 or a variant, portion or complementary sequence thereof,
k. SNP15 at position 199 of SEQ ID NO:11 or a variant, portion or complementary sequence thereof,
l. SNP16 at position 266 of SEQ ID NO:12 or a variant, portion or complementary sequence thereof,
m. SNP17 at position 216 of SEQ ID NO:13 or a variant, portion or complementary sequence thereof,
n. SNP18 at position 187 of SEQ ID NO:14 or a variant, portion or complementary sequence thereof,
o. SNP19 at position 279 of SEQ ID NO:15 or a variant, portion or complementary sequence thereof,
p. SNP20 at position 188 of SEQ ID NO:16 or a variant, portion or complementary sequence thereof,
q. SNP21 at position 165 of SEQ ID NO:17 or a variant, portion or complementary sequence thereof,
r. SNP22 at position 183 of SEQ ID NO:18 or a variant, portion or complementary sequence thereof,
s. SNP23 at position 133 and/or SNP24 at position 182 of SEQ ID NO:19 or a variant, portion or complementary sequence thereof,
t. SNP25 at position 146 and/or SNP26 position 323 of SEQ ID NO:20 or a variant, portion or complementary sequence thereof,
u. SNP27 at position 241 of SEQ ID NO:21 or a variant, portion or complementary sequence thereof,
v. SNP28 at position 236 of SEQ ID NO:22 or a variant, portion or complementary sequence thereof,
w. SNP29 at position 199 of SEQ ID NO:23 or a variant, portion or complementary sequence thereof,
x. SNP30 at position 185 of SEQ ID NO:24 or a variant, portion or complementary sequence thereof,
y. SNP31 at position 158 of SEQ ID NO:25 or a variant, portion or complementary sequence thereof,
z. SNP32 at position 143 of SEQ ID NO:26 or a variant, portion or complementary sequence thereof,
aa. SNP33 at position 157 of SEQ ID NO:27 or a variant, portion or complementary sequence thereof, and
bb. SNP34 at position 161 of SEQ ID NO:28 or a variant, portion or complementary sequence thereof, and any combination thereof.

It is a further object of the present invention to disclose the cultivated P. persimilis mite individual as defined in any of the above, wherein said variant comprises at least 90% sequence similarity to the corresponding nucleotide SEQ ID sequence.

It is a further object of the present invention to disclose the cultivated P. persimilis mite individual as defined in any of the above, wherein said P. persimilis predatory individual comprising within it's genome a polynucleotide sequence selected from SEQ ID NO:1 to SEQ ID NO:28, and any combination thereof.

It is a further object of the present invention to disclose the cultivated P. persimilis mite individual as defined in any of the above, wherein the at least one SNP is detected using primers comprising pairs of sequences selected from: SEQ ID NO:29 and SEQ ID NO:30 for detection of SNP1, SEQ ID NO:31 and SEQ ID NO:32 for detection of SNP2, SEQ ID NO:33 and SEQ ID NO:34 for detection of SNP3, SEQ ID NO:35 and SEQ ID NO:36 for detection of SNP4-5, SEQ ID NO:37 and SEQ ID NO:38 for detection of SNP6, SEQ ID NO:39 and SEQ ID NO:40 for detection of SNP7-8, SEQ ID NO:41 and SEQ ID NO:42 for detection of SNP9, SEQ ID NO:43 and SEQ ID NO:44 for detection of SNP10-11, SEQ ID NO:45 and SEQ ID NO:46 for detection of SNP12-13, SEQ ID NO:47 and SEQ ID NO:48 for detection of SNP14, SEQ ID NO:49 and SEQ ID NO:50 for detection of SNP15-16, SEQ ID NO:51 and SEQ ID NO:50 for detection of SNP16, SEQ ID NO:52 and SEQ ID NO:53 for detection of SNP17, SEQ ID NO:54 and SEQ ID NO:55 for detection of SNP18, SEQ ID NO:56 and SEQ ID NO:57 for detection of SNP19, SEQ ID NO:58 and SEQ ID NO:59 for detection of SNP20, SEQ ID NO:60 and SEQ ID NO:61 for detection of SNP21, SEQ ID NO:62 and SEQ ID NO:63 for detection of SNP22, SEQ ID NO:64 and SEQ ID NO:65 for detection of SNP23-24, SEQ ID NO:66 and SEQ ID NO:67 for detection of SNP25-26, SEQ ID NO:68 and SEQ ID NO:69 for detection of SNP27, SEQ ID NO:70 and SEQ ID NO:71 for detection of SNP28, SEQ ID NO:72 and SEQ ID NO:73 for detection of SNP29, SEQ ID NO:74 and SEQ ID NO:75 for detection of SNP30, SEQ ID NO:76 and SEQ ID NO:77 for detection of SNP31, SEQ ID NO:78 and SEQ ID NO:79 for detection of SNP32, SEQ ID NO:80 and SEQ ID NO:81 for detection of SNP33, and SEQ ID NO:82 and SEQ ID NO:83 for detection of SNP34.

It is a further object of the present invention to disclose an isolated cell of the cultivated Phytoseiulus persimilis (P. persimilis) predatory mite as defined in any of the above.

It is a further object of the present invention to disclose a Phytoseiulus persimilis rearing mite population, wherein at least 5% of the population are the cultivated P. persimilis mite individuals as defined in any of the above.

It is a further object of the present invention to disclose the Phytoseiulus persimilis rearing mite population as defined in any of the above, wherein the population further comprises a food source for the P. persimilis predatory mites, said food source comprises a non-Tetranychid arthropod as a factitious host, preferably an immobilized non-Tetranychid arthropod prey, such as on an Astigmatid prey, most preferably on an immobilized Astigmatid prey.

It is a further object of the present invention to disclose the Phytoseiulus persimilis rearing mite population as defined in any of the above, wherein the population is characterized by improved rearing properties on a non-Tetranychid arthropod prey as a food source, preferably on an immobilized non-Tetranychid arthropod prey, such as on an Astigmatid prey, most preferably on an immobilized Astigmatid prey.

It is a further object of the present invention to disclose the Phytoseiulus persimilis rearing mite population as defined in any of the above, wherein the improved rearing properties on a non-Tetranychid arthropod prey are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals characterized by less than 5% individuals comprising within their genome said at least one SNP marker.

It is a further object of the present invention to disclose the Phytoseiulus persimilis rearing mite population as defined in any of the above, wherein the population is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

It is a further object of the present invention to disclose use of a Phytoseiulus persimilis rearing mite population as defined in any of the above, for controlling a pest in a crop.

It is a further object of the present invention to disclose a method for controlling a pest in a crop comprising providing the crop with a Phytoseiulus persimilis rearing mite population as defined in any of the above.

It is a further object of the present invention to disclose device for releasing mite individuals of a Phytoseiulus persimilis predatory mite species, said device comprising a container holding a biological control composition as defined in any of the above, or a Phytoseiulus persimilis rearing mite population as defined in any of the above, wherein the device comprises an exit for mobile life stages of the P. persimilis predatory mite species, preferably an exit suitable for providing a sustained release of a number of mobile life stages.

It is a further object of the present invention to disclose use of a biological control composition as defined in any of the above, or a Phytoseiulus persimilis rearing mite population as defined in any of the above, preferably in a device as defined in any of the above, for crop protection.

It is a further object of the present invention to disclose use of a non-Tetranychid arthropod species, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, as a food source, preferably as a rearing prey, for a Phytoseiulus persimilis rearing mite population as defined in any of the above.

It is a further object of the present invention to disclose the use as defined in any of the above, wherein said use comprises applying individuals of a non-Tetranychid arthropod species to a target plant, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, or a mixture of immobilized life stages comprising eggs and mobile stages of the non-Tetranychid arthropod species.

It is a further object of the present invention to disclose a method for identifying and/or selecting Phytoseiulus persimilis (P. persimilis) predatory mite individuals characterized by improved rearing properties on a non-Tetranychid arthropod prey as a food source, preferably on an immobilized non-Tetranychid arthropod prey, such as on an Astigmatid prey, most preferably on an immobilized Astigmatid prey, wherein said method comprises detecting in the genome of the P. persimilis predatory mite individuals, the presence of a genomic haplotype comprising a single nucleotide polymorphism (SNP) marker at a position corresponding to at least one of: SNP1 at position 163 of SEQ ID NO:1 or a variant, portion or complementary sequence thereof, SNP2 at position 181 of SEQ ID NO:2 or a variant, portion or complementary sequence thereof, SNP3 at position 197 of SEQ ID NO:3 or a variant, portion or complementary sequence thereof, SNP4 at position 111 of SEQ ID NO:4 or a variant, portion or complementary sequence thereof, SNP5 at position 165 of SEQ ID NO:4 or a variant, portion or complementary sequence thereof, SNP6 at position 193 of SEQ ID NO:5 or a variant, portion or complementary sequence thereof, SNP7 at position 186 of SEQ ID NO:6 or a variant, portion or complementary sequence thereof, SNP8 at position 193 of SEQ ID NO:6 or a variant, portion or complementary sequence thereof, SNP9 at position 161 of SEQ ID NO:7 or a variant, portion or complementary sequence thereof, SNP10 at position 203 of SEQ ID NO:8 or a variant, portion or complementary sequence thereof, SNP11 at position 213 of SEQ ID NO:8 or a variant, portion or complementary sequence thereof, SNP12 at position 163 of SEQ ID NO:9 or a variant, portion or complementary sequence thereof, SNP13 at position 192 of SEQ ID NO:9 or a variant, portion or complementary sequence thereof, SNP14 at position 208 of SEQ ID NO:10 or a variant, portion or complementary sequence thereof, SNP15 at position 199 of SEQ ID NO:11 or a variant, portion or complementary sequence thereof, SNP16 at position 266 of SEQ ID NO:12 or a variant, portion or complementary sequence thereof, SNP17 at position 216 of SEQ ID NO:13 or a variant, portion or complementary sequence thereof, SNP18 at position 187 of SEQ ID NO:14 or a variant, portion or complementary sequence thereof, SNP19 at position 279 of SEQ ID NO:15 or a variant, portion or complementary sequence thereof, SNP20 at position 188 of SEQ ID NO:16 or a variant, portion or complementary sequence thereof, SNP21 at position 165 of SEQ ID NO:17 or a variant, portion or complementary sequence thereof, SNP22 at position 183 of SEQ ID NO:18 or a variant, portion or complementary sequence thereof, SNP23 at position 133 of SEQ ID NO:19 or a variant, portion or complementary sequence thereof, SNP24 at position 182 of SEQ ID NO:19 or a variant, portion or complementary sequence thereof, SNP25 at position 146 of SEQ ID NO:20 or a variant, portion or complementary sequence thereof, SNP26 at position 323 of SEQ ID NO:20 or a variant, portion or complementary sequence thereof, SNP27 at position 241 of SEQ ID NO:21 or a variant, portion or complementary sequence thereof, SNP28 at position 236 of SEQ ID NO:22 or a variant, portion or complementary sequence thereof, SNP29 at position 199 of SEQ ID NO:23 or a variant, portion or complementary sequence thereof, SNP30 at position 185 of SEQ ID NO:24 or a variant, portion or complementary sequence thereof, SNP31 at position 158 of SEQ ID NO:25 or a variant, portion or complementary sequence thereof, SNP32 at position 143 of SEQ ID NO:26 or a variant, portion or complementary sequence thereof, SNP33 at position 157 of SEQ ID NO:27 or a variant, portion or complementary sequence thereof, SNP34 at position 161 of SEQ ID NO:28 or a variant, portion or complementary sequence thereof, and any combination thereof.

It is a further object of the present invention to disclose the method as defined in any of the above, wherein said variant comprises at least 90% similarity to the corresponding nucleotide SEQ ID sequence.

It is a further object of the present invention to disclose the method as defined in any of the above, wherein said Phytoseiulus predatory individual comprising a polynucleotide genomic sequence selected from SEQ ID NO:1 to SEQ ID NO:28, and any combination thereof.

It is a further object of the present invention to disclose the method as defined in any of the above, wherein the detection is performed using a primer pair selected from: SEQ ID NO:29 and SEQ ID NO:30 for detection of SNP1, SEQ ID NO:31 and SEQ ID NO:32 for detection of SNP2, SEQ ID NO:33 and SEQ ID NO:34 for detection of SNP3, SEQ ID NO:35 and SEQ ID NO:36 for detection of SNP4-5, SEQ ID NO:37 and SEQ ID NO:38 for detection of SNP6, SEQ ID NO:39 and SEQ ID NO:40 for detection of SNP7-8, SEQ ID NO:41 and SEQ ID NO:42 for detection of SNP9, SEQ ID NO:43 and SEQ ID NO:44 for detection of SNP10-11, SEQ ID NO:45 and SEQ ID NO:46 for detection of SNP12-13, SEQ ID NO:47 and SEQ ID NO:48 for detection of SNP14, SEQ ID NO:49 and SEQ ID NO:50 for detection of SNP15-16, SEQ ID NO:51 and SEQ ID NO:50 for detection of SNP16, SEQ ID NO:52 and SEQ ID NO:53 for detection of SNP17, SEQ ID NO:54 and SEQ ID NO:55 for detection of SNP18, SEQ ID NO:56 and SEQ ID NO:57 for detection of SNP19, SEQ ID NO:58 and SEQ ID NO:59 for detection of SNP20, SEQ ID NO:60 and SEQ ID NO:61 for detection of SNP21, SEQ ID NO:62 and SEQ ID NO:63 for detection of SNP22, SEQ ID NO:64 and SEQ ID NO:65 for detection of SNP23-24, SEQ ID NO:66 and SEQ ID NO:67 for detection of SNP25-26, SEQ ID NO:68 and SEQ ID NO:69 for detection of SNP27, SEQ ID NO:70 and SEQ ID NO:71 for detection of SNP28, SEQ ID NO:72 and SEQ ID NO:73 for detection of SNP29, SEQ ID NO:74 and SEQ ID NO:75 for detection of SNP30, SEQ ID NO:76 and SEQ ID NO:77 for detection of SNP31, SEQ ID NO:78 and SEQ ID NO:79 for detection of SNP32, SEQ ID NO:80 and SEQ ID NO:81 for detection of SNP33, SEQ ID NO:82 and SEQ ID NO:83 for detection of SNP34 and any combination of primer pairs.

It is a further object of the present invention to disclose the method as defined in any of the above, wherein the improved rearing properties on a non-Tetranychid arthropod prey are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals lacking the at least one SNP and or the polynucleotide sequence selected from SEQ ID NO:1 to SEQ ID NO:28.

It is a further object of the present invention to disclose a method for identifying and/or selecting a cultivated rearing population of Phytoseiulus persimilis (P. persimilis) predatory mite individuals characterized by improved rearing properties on a non-Tetranychid arthropod prey as a food source, preferably on an immobilized non-Tetranychid arthropod prey, such as on an Astigmatid prey, most preferably on an immobilized Astigmatid prey, wherein said method comprises detecting in the genome of at least 5% of said P. persimilis individuals at least one Single Nucleotide Polymorphism (SNP) marker selected from:
a. SNP of G at position 34739 of SEQ ID NO:84 or a variant, portion or complementary sequence thereof,
b. at least one SNP selected from C at position 105452, G at position 109775, A at position 115020, T at position 115073, C at position 125940, G at position 131548, G at position 131555, of SEQ ID NO:85 or a variant, portion or complementary sequence thereof,
c. at least one SNP selected from A at position 99333, G at position 99595, T at position 99605, A at position 115325, C at position 115354, of SEQ ID NO:86 or a variant, portion or complementary sequence thereof,
d. at least one SNP selected from A at position 4508, A at position 11697, C at position 12229, A at position 13135, C at position 32979, G at position 38081, C at position 38220 of SEQ ID NO:87 or a variant, portion or complementary sequence thereof,
e. at least one SNP selected from A at position 30913, C at position 34950, C at position 38409, A at position 38458, T at position 42427, G at position 42604, T at position 43158 of SEQ ID NO:88 or a variant, portion or complementary sequence thereof,
f. SNP of G at position 1032 of SEQ ID NO:89 or a variant, portion or complementary sequence thereof,
g. SNP of A at position 173977 and/or SNP of T at position 228195 of SEQ ID NO:90 or a variant, portion or complementary sequence thereof, and
h. at least one SNP selected from C at position 857, T at position 15689, C at position 40135, G at position 49492 of SEQ ID NO:91 or a variant, portion or complementary sequence thereof.

It is a further object of the present invention to disclose the method as defined in any of the above, wherein said variant comprises at least 90% sequence similarity to the corresponding SEQ ID nucleotide sequence.

It is a further object of the present invention to disclose the method as defined in any of the above, wherein at least 5.5%, such as at least 10%, 20%, 30%, 40%, 50%, 60%, 70%, 80%, at least 90% or 100% of said P. persimilis predatory individuals of said cultivated population comprise said at least one SNP marker.

It is a further object of the present invention to disclose the method as defined in any of the above, wherein the at least 5% of said P. persimilis predatory individuals comprise within their genome a polynucleotide genomic sequence selected from SEQ ID NO:1-28 and SEQ ID NO:84-91, and any combination thereof.

It is a further object of the present invention to disclose the method as defined in any of the above, wherein the at least 5% of said P. persimilis predatory individuals comprise within their genome a polynucleotide genomic sequence selected from SEQ ID NO:1 to SEQ ID NO:91, and any combination thereof.

It is a further object of the present invention to disclose the method as defined in any of the above, wherein the cultivated rearing population of P. persimilis predatory mite individuals is characterized by improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

It is a further object of the present invention to disclose the method as defined in any of the above, wherein the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

It is a further object of the present invention to disclose a method for obtaining a Phytoseiulus persimilis rearing mite population characterized by improved rearing properties on a non-Tetranychid arthropod prey as a factious host, preferably on an immobilized non-Tetranychid arthropod prey, such as on an Astigmatid prey, most preferably on an immobilized Astigmatid prey, wherein at least 5% of the population are the cultivated P. persimilis mite individuals as defined in any of the above, said method comprises steps of:
a. providing a rearing population of a P. persimilis predatory mite species, the rearing population comprising individuals of the P. persimilis species, reared on a suitable food source for the Phytoseiulus individuals, said food source comprising a prey species selected from the Tetranychidae species;
b. providing a population of individuals of preselected non-Tetranychid arthropod species, preferably an Astigmatid mite species, most preferably an immobilized Astigmatid mite species having immobilized life stages comprising immobilized eggs;
c. rearing the P. persimilis individuals on the preselected non-Tetranychid arthropod species as a food source;
d. screening the genome of a sample of P. persimilis individuals for the presence of at least 5% of at least one SNP selected from:
   i SNP of G at position 34739 of SEQ ID NO:84 or a variant, portion or complementary sequence thereof,
   ii at least one SNP selected from C at position 105452, G at position 109775, A at position 115020, T at position 115073, C at position 125940, G at position 131548, G at position 131555, of SEQ ID NO:85 or a variant, portion or complementary sequence thereof,
   iii at least one SNP selected from A at position 99333, G at position 99595, T at position 99605, A at position 115325, C at position 115354, of SEQ ID NO:86 or a variant, portion or complementary sequence thereof,
   iv at least one SNP selected from A at position 4508, A at position 11697, C at position 12229, A at position 13135, C at position 32979, G at position 38081, C at position 38220 of SEQ ID NO:87 or a variant, portion or complementary sequence thereof,
   v at least one SNP selected from A at position 30913, C at position 34950, C at position 38409, A at position 38458, T at position 42427, G at position 42604, T at position 43158 of SEQ ID NO:88 or a variant, portion or complementary sequence thereof,
   vi SNP of G at position 1032 of SEQ ID NO:89 or a variant, portion or complementary sequence thereof,
   vii SNP of A at position 173977 and/or SNP of T at position 228195 of SEQ ID NO:90 or a variant, portion or complementary sequence thereof, and
   viii at least one SNP selected from C at position 857, T at position 15689, C at position 40135, G at position 49492 of SEQ ID NO:91 or a variant, portion or complementary sequence thereof;
e. optionally, alternatingly rearing selected Phytoseiulus individuals in a sequence of:
   rearing for at least 2, such as between 5 and 50, generations while using a food source comprising the preselected non-Tetranychid arthropod species;
   rearing for at least 2, such as between 5 and 50, generations while using a food source comprising the prey species selected from the Tetranychidae
   screening the genome of a sample of P. persimilis individuals for the presence of at least 5% of the at least one SNP.

It is a further object of the present invention to disclose the method as defined in any of the above, further comprises steps of:
a. separating eggs and/or juvenile developmental stages such as larvae and/or nymphs from the preselected non-Tetranychid arthropod prey rearing population;
b. mixing the separated eggs and/or juvenile developmental stages such as larvae and/or nymphs with a carrier material, such as a carrier material selected from sawdust, wheat bran, buckwheat husks, rice husks or millet husks, or comprising a mixture thereof;
c. freezing the mixture of step (b); and
d. rearing the predatory mite individuals on the mixture as a food source.

It is a further object of the present invention to disclose the method as defined in any of the above, wherein the provided rearing population is a population composed of a number of sub-populations, wherein said sub-populations are from distinct sources, such as from distinct production populations and/or from natural populations isolated from distinct geographical locations.

It is a further object of the present invention to disclose the method as defined in any of the above, wherein the provided rearing population comprises at least 100 individuals, such as between 200 and 5000 individuals, preferably between 500 and 1500 individuals.

It is a further object of the present invention to disclose the method as defined in any of the above, wherein the sample for genome screening comprises at least 50 individuals, such as between 100 and 300 individuals.

It is a further object of the present invention to disclose the method as defined in any of the above, wherein the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

It is a further object of the present invention to disclose an isolated nucleic acid sequence comprising at least 90% similarity to a polynucleotide sequence selected from SEQ ID NO:1 to SEQ ID NO:91 or a portion thereof and any combination thereof.

It is a further object of the present invention to disclose use of a nucleic acid sequence selected from SEQ ID NO:84 to SEQ ID NO:91 or a portion thereof to identify a rearing population of Phytoseiulus persimilis (P. persimilis) predatory mite individuals characterized by improved rearing properties on a non-Tetranychid arthropod prey as a factitious host, preferably on an immobilized non-Tetranychid arthropod prey, such as on an Astigmatid prey, most preferably on an immobilized Astigmatid prey, wherein at least 5% of said P. persimilis individuals comprise within their genome at least one Single Nucleotide Polymorphism (SNP) marker selected from:
a. SNP of G at position 34739 of SEQ ID NO:84 or a variant, portion or complementary sequence thereof,
b. at least one SNP selected from C at position 105452, G at position 109775, A at position 115020, T at position 115073, C at position 125940, G at position 131548, G at position 131555, of SEQ ID NO:85 or a variant, portion or complementary sequence thereof,
c. at least one SNP selected from A at position 99333, G at position 99595, T at position 99605, A at position 115325, C at position 115354, of SEQ ID NO:86 or a variant, portion or complementary sequence thereof,
d. at least one SNP selected from A at position 4508, A at position 11697, C at position 12229, A at position 13135, C at position 32979, G at position 38081, C at position 38220 of SEQ ID NO:87 or a variant, portion or complementary sequence thereof,
e. at least one SNP selected from A at position 30913, C at position 34950, C at position 38409, A at position 38458, T at position 42427, G at position 42604, T at position 43158 of SEQ ID NO:88 or a variant, portion or complementary sequence thereof,
f. SNP of G at position 1032 of SEQ ID NO:89 or a variant, portion or complementary sequence thereof,
g. SNP of A at position 173977 and/or SNP of T at position 228195 of SEQ ID NO:90 or a variant, portion or complementary sequence thereof, and
h. at least one SNP selected from C at position 857, T at position 15689, C at position 40135, G at position 49492 of SEQ ID NO:91 or a variant, portion or complementary sequence thereof.

It is a further object of the present invention to disclose the use as defined in any of the above, wherein said variant comprises at least 90% sequence similarity to the corresponding SEQ ID nucleotide sequence.

It is a further object of the present invention to disclose the use as defined in any of the above, wherein at least 5.5%, such as at least 10%, 20%, 30%, 40%, 50%, 60%, 70%, 80%, at least 90% or 100% of said P. persimilis predatory individuals of said population comprise said at least one SNP marker.

It is a further object of the present invention to disclose the use as defined in any of the above, wherein the rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

It is a further object of the present invention to disclose use of a nucleic acid sequence selected from SEQ ID NO:1 to SEQ ID NO:28 or a portion thereof to identify Phytoseiulus persimilis (P. persimilis) predatory mite individuals characterized by improved rearing properties on a non-Tetranychid arthropod prey as a factitious host, preferably on an immobilized non-Tetranychid arthropod prey, such as on an Astigmatid prey, most preferably on an immobilized Astigmatid prey, as compared to Phytoseiulus persimilis (P. persimilis) predatory mite individuals lacking the at least one sequence.

It is a further object of the present invention to disclose the use as defined in any of the above, wherein the improved rearing properties on a non-Tetranychid arthropod prey are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals lacking the at least one SNP and or the polynucleotide sequence selected from SEQ ID NO:1 to SEQ ID NO:28.

It is a further object of the present invention to disclose an isolated nucleic acid primer pair sequence comprising at least 90% similarity to a polynucleotide sequence selected from SEQ ID NO:29 to SEQ ID NO:83 and any combination thereof.

It is a further object of the present invention to disclose use of the isolated nucleic acid primer pair sequence as defined above, for identifying and/or selecting and/or obtaining Phytoseiulus persimilis (P. persimilis) predatory mite individual characterized by improved rearing properties on a non-Tetranychid arthropod prey as a factitious host, preferably on an immobilized non-Tetranychid arthropod prey, such as on an Astigmatid prey, most preferably on an immobilized Astigmatid prey, and/or for obtaining the Phytoseiulus persimilis (P. persimilis) predatory mite population as defined in any of the above, or mite individual as defined in any of the above.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings; wherein:

FIG. 1 is a schematic representation of P+ line genetic marker denomination;

FIG. 2 is graphically presenting the rate of P+ allele in P+, P− and Alternation P. persimilis populations based on population-wide high-throughput genomic DNA sequencing data;

FIG. 3 is graphically alternatively presenting results of population-wide high-throughput genomic DNA sequencing showing percentage of similarity to P+ reference allele tested in different sampling times (events) and different population sources (P+, P− and Alternation). The top part of the graph shows average results of the different markers tested, and in the bottom part of the graph, each dot represents the result for a specific marker for each sample; and

FIG. 4 is graphically presenting Lambda (λ) values of various P+‘continuous’ and ‘Alternating’ population lines.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

In the description of the present application, each of the verbs, “comprise” “include” and “have”, and conjugates thereof, are used to indicate that the object or objects of the verb are not necessarily a complete listing of components, elements or parts of the subject or subjects of the verb. Unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of an embodiment as described. In addition, the word “or” is considered to be the inclusive “or” rather than the exclusive or, and indicates at least one of, or any combination of items it conjoins.

The two-spotted spider mite, Tetranychus urticae Koch, is the major spider mite pest of ornamental plants and vegetable crops grown in greenhouses. Furthermore, this ubiquitous spider mite is a serious pest of numerous ornamental plants in home landscapes and is of considerable importance as a pest of food and fiber crops throughout the world (van de Vrie et al., 1972). The predacious phytoseiid mite Phytoseiulus persimilis (P. persimilis) is the major species used to control twos-potted spider mites in greenhouse as well as open field crops.

Phytoseiulus persimilis is a predatory mite, which specializes on a diet of spider mites. Spider mites are vegetarian mites (phytophagous mites) and therefore require rearing on plants, which involves complex operations and high rearing costs.

Rearing Phytoseiulus persimilis on other preys (e.g. non-phytophagous preys), which are cheaper to produce, would be therefore highly desirable. The alternative prey mites could be, for example, Astigmatic mites that feed on stored products and are therefore significantly cheaper to produce (see WO2019171374 and WO2021044404 incorporated herein by their entirety).

It is herein emphasized that up until the current invention there was no availability or publication of sequence information relating to the nuclear genome (or DNA) of P. persimilis, which can be used as reference or benchmark for assessing genetic variations unique for desirable traits such as development and reproduction on a non-Tetranychid factitious host. There is very limited information in NCBI, mostly of few partial genes.

The present invention provides for the first time whole genome sequencing information of a P. persimilis mite species. Based on the herein generated reference P. persimilis genome, novel and unique genomic haplotypes and molecular markers were identified for P. persimilis mites characterized by the desirable trait of development and reproduction (i.e. improved rearing) on a non-Tetranychid arthropod species (e.g. Astigmatic mite species, e.g. Carpoglyphus lactis).

The approach used by the present invention for performing the genetic analysis was de novo assembly of whole-genome shotgun sequencing data. This technique enables assembly of a novel genome where there is no reference genome sequence available for alignment. Sequence reads are assembled as contigs, and the coverage quality of the de novo assembly relates to the size and continuity of the contigs (i.e. the number of gaps in the data). This population-wide high-throughput genomic DNA sequencing technology enables fast and accurate characterization of a predetermined species.

According to embodiments of the present invention, the genetic profiling results of P. persimilis present newly identified haplotypes and genetic markers comprising combinations of genetic variants unique for and associated with P. persimilis populations, which exhibit significantly improved development and reproduction parameters when preying on non-Tetranychid arthropod species, such as Astigmatid mite species (P+ line). It should be emphasized that these unique genetic markers (Single Nucleotide Polymorphisms [SNPs]) were absent in any P. persimilis population tested that does not exhibit the trait of improved development and reproduction on non-Tetranychid arthropod species (P− line).

According to some embodiments of the present invention, the improved rearing properties on a non-Tetranychid arthropod prey include parameters such as increased daily reproduction rate (λ), increased daily oviposition rate, high percentage of female individuals capable of reproduction on the non-Tetranychid arthropod prey, and enhanced juvenile survival rate, as compared to a non-P+ population growing on non-Tetranychid as the sole prey or food source.

It is emphasized that the P. persimilis population of the present invention comprising the herein identified unique genomic markers, exhibits a similar and even better ability to control their natural prey, spider mites.

According to one embodiment, the present invention provides a biological control composition comprising a cultivated rearing population of Phytoseiulus persimilis (P. persimilis) predatory mite individuals, and optionally a carrier.

According to a further embodiment, at least 5% of the P. persimilis individuals of the population comprise within their genome at least one Single Nucleotide Polymorphism (SNP) marker selected from: SNP at position 34739 of SEQ ID NO:84 or a variant, portion or complementary sequence thereof, at least one SNP at a position selected from 105452, 109775, 115020, 115073, 125940, 131548 and 131555, of SEQ ID NO:85 or a variant, portion or complementary sequence thereof, at least one SNP at a position selected from 99333, 99595, 99605, 115325 and 115354, of SEQ ID NO:86 or a variant, portion or complementary sequence thereof, at least one SNP at a position selected from 4508, 11697, 12229, 13135, 32979, 38081, 38220, of SEQ ID NO:87 or a variant, portion or complementary sequence thereof, at least one SNP at a position selected from 30913, 34950, 38409, 38458, 42427, 42604 and 43158 of SEQ ID NO:88 or a variant, portion or complementary sequence thereof, SNP at position 1032 of SEQ ID NO:89 or a variant, portion or complementary sequence thereof, SNP at position 173977 and/or SNP at position 228195 of SEQ ID NO:90 or a variant, portion or complementary sequence thereof, and at least one SNP at a position selected from 857, 15689, 40135 and 49492 of SEQ ID NO:91 or a variant, portion or complementary sequence thereof.

It is within scope of the present invention that the variant comprises at least 90% sequence similarity to the corresponding SEQ ID nucleotide sequence.

It is further within the scope of the present invention that at least 5.5%, such as at least 10%, 20%, 30%, 40%, 50%, 60%, 70%, 80%, at least 90% or 100% of said P. persimilis predatory individuals of said cultivated population comprise said at least one SNP marker.

According to further aspects of the present invention, the at least 5% of the P. persimilis predatory individuals comprise within their genome a polynucleotide genomic sequence selected from SEQ ID NO:1-28 and SEQ ID NO:84-91, and any combination thereof.

According to further aspects of the present invention, the at least 5% of the P. persimilis predatory individuals comprise within their genome a polynucleotide genomic sequence selected from SEQ ID NO:1 to SEQ ID NO:91, and any combination thereof.

According to main aspects of the present invention, the cultivated rearing population of P. persimilis predatory mite individuals is characterized by improved rearing properties on a non-Tetranychid arthropod prey as a factious host or as a food source, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

According to a further embodiment, the present invention provides a method for identifying and/or selecting Phytoseiulus persimilis (P. persimilis) predatory mite individuals characterized by improved rearing properties on a non-Tetranychid arthropod prey as a factitious host, preferably on an immobilized non-Tetranychid arthropod prey, such as on an Astigmatid prey, most preferably on an immobilized Astigmatid prey.

Unless defined otherwise, all technical and scientific terms used herein have the same meaning as commonly understood to one of ordinary skill in the art to which the presently disclosed subject matter belongs.

As used herein the term “about” denotes ±10% of the defined amount or measure or value.

As used herein the term “similar” denotes a correspondence or resemblance range of about +/−20%, particularly +/−15%, more particularly about +/−10% and even more particularly about +/−5%.

As used herein the term “average” refers to the mean value as obtained by measuring a predetermined parameter in each individual of a certain population and calculating the mean value according to the number of individuals in said population.

The term “consists essentially of” (and grammatical variants thereof), as applied to a polynucleotide sequence of this invention, means a polynucleotide sequence that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together.

A marker or genomic or genetic sequence is “associated with” a trait, phenotype or characteristic when it is linked to it and when the presence of the marker is an indicator of whether and/or to what extent the desired trait or trait form will occur in an organism (mite species)/germplasm comprising the marker. Similarly, a marker is “associated with” an allele when it is linked to it and when the presence of the marker is an indicator of whether the allele is present in an organism (mite species)/germplasm comprising the marker. For example, in the context of the current invention, a genetic marker associated with improved rearing on a non-Tetranychid arthropod prey, refers to a marker whose presence or absence can be used to predict whether and/or to what extent a P. persimilis predatory mite will have the characteristic of developing and reproducing on a non-Tetranychid arthropod prey, such as an immobilized Astigmatid prey (e.g. any SNP allele as described in Table 1 are “associated with” the characteristic of the P. persimilis).

The term “rearing”, as used herein, broadly refers to breeding, reproducing, multiplication, ovipositing, surviving and growing of individuals, and includes the propagation and increase of a Phytoseiulus population by sexual reproduction.

The first composition and method of the invention is directed to rearing a predatory mite from the family Phytoseiulus, specifically of the P. persimilis species, characterized by a specific haplotype (at least one SNP) associated with the trait of improved reproducing on a non-Tetranychid arthropod prey, such as an immobilized Astigmatid prey.

As used herein, the term “improved rearing on a non-Tetranychid arthropod prey” or “improved rearing properties on a non-Tetranychid arthropod prey” refers in the context of the current invention to a phenotypic characteristic of individual P. persimilis predatory mites (cultivated P. persimilis) carrying the genomic haplotype comprising at least one of the genomic markers as described in Tables 2, 3, 5 and 6 (or a complementary sequence thereof), and/or to a population P. persimilis predatory mite individuals (cultivated population) wherein at least 5% of the individuals in the population carry the genomic haplotype comprising at least one of the genomic markers as described in Tables 1, 4 and 7 (or a complementary sequence thereof). The phenotypic characteristic of the P. persimilis predatory mites is defined by exhibiting enhanced development and reproduction while being fed on a non-Tetranychid arthropod prey, such as Astigmatid mite species, preferably immobilized Astigmatid mite species, e.g. comprising immobilized eggs. This phenotypic characteristic or trait is as compared to a population of P. persimilis predatory mite individuals, wherein less than 5% of the individuals in the population carry the genomic haplotype comprising at least one of the genomic markers as described in Tables 1, 4 and 7 (or a complementary sequence thereof).

In some aspects, the improved rearing phenotype or trait refers to enhanced development and reproduction non-Tetranychid arthropod prey as compared to P. persimilis predatory mites lacking the genomic haplotype comprising at least one of the genomic markers as described in Tables 2, 3, 5 and 6 (or a complementary sequence thereof). In further aspects of the present invention, the improved rearing on a non-Tetranychid arthropod prey include properties such as daily reproduction rate (λ), daily oviposition rate, survival rate, mortality rate, juvenile and/or female survival rate, percentage of female individuals reproducing on said prey, and predatory behavior towards a Tetranychidae species, while preying on a non-Tetranychid arthropod prey.

The term “population-wide high-throughput genomic DNA sequencing” refers generally to any high-throughput sequencing technology, such as Next Generation Sequencing (NGS) method, population-wide whole-genome shotgun sequencing technology and any large-scale genetic analysis technology.

As used herein, the term “variant” with respect to a sequence, e.g., a polypeptide or nucleic acid sequence such as—for example—a genomic region nucleotide sequence of the invention, especially, a polynucleotide sequence flanking both sides of the SNP nucleotide position (towards the 5′ and 3′ ends) within genomic regions, contigs or SNPs as listed in Tables 1-7 (or complementary sequences thereof, or having SEQ ID NO:1-91), is intended to mean substantially similar sequences. For nucleotide sequences comprising an open reading frame, variants include those sequences that, because of the degeneracy of the genetic code, encode the identical amino acid sequence of the native protein. Allelic variants can be identified with the use of well-known molecular biology techniques, as, for example, with polymerase chain reaction (PCR), sequencing techniques and hybridization techniques. Variant nucleotide sequences also include synthetically derived nucleotide sequences, such as those generated, for example, by using site-directed mutagenesis. Generally, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity or sequence similarity to the nucleotide sequence of SEQ ID NO: SEQ ID NO: 1-91.

The term “cultivated” is herein understood to refer to a mite or mite population, that is no longer in the natural state but has been developed by human care and for human use (e.g. biological control purposes) and/or growing purposes and/or consumption. The term “cultivated” is further understood to exclude those wild-type species, which comprise the trait being subject of this invention as a natural trait and/or part of their natural genetics.

A cultivated mite population as used herein refers to a P. persimilis population in which at least 5% of its individuals comprise within their genome at least one Single Nucleotide Polymorphism (SNP) marker selected from:
a. SNP at position 34739 of SEQ ID NO:84 or a variant, portion or complementary sequence thereof,
b. at least one SNP at a position selected from 105452, 109775, 115020, 115073, 125940, 131548 and 131555, of SEQ ID NO:85 or a variant, portion or complementary sequence thereof,
c. at least one SNP at a position selected from 99333, 99595, 99605, 115325 and 115354, of SEQ ID NO:86 or a variant, portion or complementary sequence thereof,
d. at least one SNP at a position selected from 4508, 11697, 12229, 13135, 32979, 38081, 38220, of SEQ ID NO:87 or a variant, portion or complementary sequence thereof,
e. at least one SNP at a position selected from 30913, 34950, 38409, 38458, 42427, 42604 and 43158 of SEQ ID NO:88 or a variant, portion or complementary sequence thereof,
f. SNP at position 1032 of SEQ ID NO:89 or a variant, portion or complementary sequence thereof,
g. SNP at position 173977 and/or SNP at position 228195 of SEQ ID NO:90 or a variant, portion or complementary sequence thereof, and
h. at least one SNP at a position selected from 857, 15689, 40135 and 49492 of SEQ ID NO:91 or a variant, portion or complementary sequence thereof.

It is within the scope of the present invention that a cultivated P. persimilis population includes within its meaning mixed cultivated and non-cultivated P. persimilis populations, “Alternating” populations, “continuous” populations and any P+ type population as described herein characterized in that at least 5% of its individuals comprise within their genome at least one Single Nucleotide Polymorphism (SNP) marker selected from:
a. SNP at position 34739 of SEQ ID NO:84 or a variant, portion or complementary sequence thereof,
b. at least one SNP at a position selected from 105452, 109775, 115020, 115073, 125940, 131548 and 131555, of SEQ ID NO:85 or a variant, portion or complementary sequence thereof,
c. at least one SNP at a position selected from 99333, 99595, 99605, 115325 and 115354, of SEQ ID NO:86 or a variant, portion or complementary sequence thereof,
d. at least one SNP at a position selected from 4508, 11697, 12229, 13135, 32979, 38081, 38220, of SEQ ID NO:87 or a variant, portion or complementary sequence thereof,
e. at least one SNP at a position selected from 30913, 34950, 38409, 38458, 42427, 42604 and 43158 of SEQ ID NO:88 or a variant, portion or complementary sequence thereof,
f. SNP at position 1032 of SEQ ID NO:89 or a variant, portion or complementary sequence thereof,
g. SNP at position 173977 and/or SNP at position 228195 of SEQ ID NO:90 or a variant, portion or complementary sequence thereof, and
h. at least one SNP at a position selected from 857, 15689, 40135 and 49492 of SEQ ID NO:91 or a variant, portion or complementary sequence thereof.

According to main embodiments, the cultivated rearing population of P. persimilis predatory mite individuals is characterized by improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

It is further within the scope that the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

A cultivated P. persimilis individual mite refers herein to a Phytoseiulus persimilis (P. persimilis) mite individual comprising within its genome at least one single nucleotide polymorphism (SNP) marker selected from:
a. SNP1 at position corresponding to position 163 of SEQ ID NO:1 or a variant, portion or complementary sequence thereof,
b. SNP2 at position 181 of SEQ ID NO:2 or a variant, portion or complementary sequence thereof,
c. SNP3 at position 197 of SEQ ID NO:3 or a variant, portion or complementary sequence thereof,
d. SNP4 at position 111 and/or SNP5 at position 165 of SEQ ID NO:4 or a variant, portion or complementary sequence thereof,
e. SNP6 at position 193 of SEQ ID NO:5 or a variant, portion or complementary sequence thereof,
f. SNP7 at position 186 and/or SNP8 at position 193 of SEQ ID NO:6 or a variant, portion or complementary sequence thereof,
g. SNP9 at position 161 of SEQ ID NO:7 or a variant, portion or complementary sequence thereof,
h. SNP10 at position 203 and/or SNP11 at position 213 of SEQ ID NO:8 or a variant, portion or complementary sequence thereof,
i. SNP12 at position 163 and/or SNP13 at position 192 of SEQ ID NO:9 or a variant, portion or complementary sequence thereof,
j. SNP14 at position 208 of SEQ ID NO:10 or a variant, portion or complementary sequence thereof,
k. SNP15 at position 199 of SEQ ID NO:11 or a variant, portion or complementary sequence thereof,
l. SNP16 at position 266 of SEQ ID NO:12 or a variant, portion or complementary sequence thereof,
m. SNP17 at position 216 of SEQ ID NO:13 or a variant, portion or complementary sequence thereof,
n. SNP18 at position 187 of SEQ ID NO:14 or a variant, portion or complementary sequence thereof,
o. SNP19 at position 279 of SEQ ID NO:15 or a variant, portion or complementary sequence thereof,
p. SNP20 at position 188 of SEQ ID NO:16 or a variant, portion or complementary sequence thereof,
q. SNP21 at position 165 of SEQ ID NO:17 or a variant, portion or complementary sequence thereof,
r. SNP22 at position 183 of SEQ ID NO:18 or a variant, portion or complementary sequence thereof,
s. SNP23 at position 133 and/or SNP24 at position 182 of SEQ ID NO:19 or a variant, portion or complementary sequence thereof,
t. SNP25 at position 146 and/or SNP26 position 323 of SEQ ID NO:20 or a variant, portion or complementary sequence thereof,
u. SNP27 at position 241 of SEQ ID NO:21 or a variant, portion or complementary sequence thereof,
v. SNP28 at position 236 of SEQ ID NO:22 or a variant, portion or complementary sequence thereof,
w. SNP29 at position 199 of SEQ ID NO:23 or a variant, portion or complementary sequence thereof,
x. SNP30 at position 185 of SEQ ID NO:24 or a variant, portion or complementary sequence thereof,
y. SNP31 at position 158 of SEQ ID NO:25 or a variant, portion or complementary sequence thereof,
z. SNP32 at position 143 of SEQ ID NO:26 or a variant, portion or complementary sequence thereof,
aa. SNP33 at position 157 of SEQ ID NO:27 or a variant, portion or complementary sequence thereof, and
bb. SNP34 at position 161 of SEQ ID NO:28 or a variant, portion or complementary sequence thereof, and any combination thereof.

The cultivated P. persimilis individual mite is capable of rearing and reproducing on a non-Tetranychid arthropod prey as a food source (i.e. sole food source), preferably on an immobilized non-Tetranychid arthropod prey, such as on an Astigmatid prey, most preferably on an immobilized Astigmatid prey.

The term “crop”, as used herein, refers to a plant of economic importance and/or a men-grown plant.

In a particular embodiment, the plant or crop is selected from the group consisting of vegetables (including cucumber, pepper, eggplant, zucchini, melon, watermelon, tomato, leaf vegetables like lettuce or rocket, potato, beans and onion), orchards, vineyard, olive trees, citrus trees, stone fruit trees, berries, ornamental plants, including pot plants and cut flowers, aromatic plants, and plants for pharmaceutic use like Cannabis. In a more particular embodiment, the plant or crop is selected from the group consisting of pepper, tomato, cucumber and chrysanthemums.

The term “controlled release” refers hereinafter to slow release, sustained-release, rapid release, designed to release in a prolonged controlled mode or fashion. In the context of the present invention, it refers to predatory mite release to the crop plant gradually over a specified period of time, e.g. throughout the day or over a week or weeks.

The term “device” or “container” refers hereafter to an apparatus, a unit, a device, a compartment, a member, a strip, sachet, packet, pouch, pocket, sack, a bottle or a bag or any other housing or means for holding or rearing beneficial insects or predatory mites available or known in the art, mite individuals or a mite composition or population. In the context of the present invention, such a container may comprise (i) Phytoseiulus predatory mites, (ii) Phytoseiulus predatory mites with a factitious host (dead Astigmatid mite life-stages or other non-Tetranychid arthropod prey) (iii) a non-Tetranychid arthropod prey, preferably an immobilized non-Tetranychid arthropod prey, such as a non-phytophagous prey, preferably an Astigmatid prey, most preferably an immobilized Astigmatid prey, such as an immobilized Astigmatid prey, in particular a Carpoglyhus prey, having immobilized life stages comprising immobilized eggs, and (iv) optionally a carrier.

The term “slow release system” or a “device” or a “container” may further refer hereafter to a sachet-type release system, e.g. a sachet, packet, pouch, pocket, sack, a bottle or a bag or any other device or means for releasing the composition or formulation of the present invention. In the context of the present invention, such a composition may comprise (i) Phytoseiulus predatory mites, (ii) Phytoseiulus predatory mites with a factitious host (dead Astigmatid mite life-stages or other non-Tetranychid arthropod prey) (iii) a non-Tetranychid arthropod prey, preferably an immobilized non-Tetranychid arthropod prey, such as a non-phytophagous prey, preferably an Astigmatid prey, most preferably an immobilized Astigmatid prey, such as an immobilized Astigmatid prey, in particular a Carpoglyhus prey, having immobilized life stages comprising immobilized eggs, and (iv) optionally a carrier. It is further included within the scope of the present invention that such a system or container refers to an apparatus, a unit, a device, a compartment, a member, strip or housing for slow release of beneficial insects or predatory mites available or known in the art, which gradually releases the beneficial insects or predatory mites. Having knowledge of such systems, the skilled person will understand that such a gradual release is opposed to immediate release.

It is also within the scope of the present invention that the Phytoseiulus predatory mite rearing or releasing system may be of any suitable type. In general the mite rearing or releasing system may comprise a container suitable for holding the individuals of the Phytoseiulus predatory mite (e.g. P. persimilis) and individuals of the factitious host mite (e.g. dead or immobilized Asigmatid eggs or juvenile stages). A container for releasing the predatory mites may comprise an opening and/or means for generating an exit opening for mobile stages of the Phytoseiulus predatory mite. Releasing systems of this type are known to the skilled person and/or are commercially available on the market, e.g. sachet-type releasing systems and other suitable types of releasing systems, which are included within the scope of the present invention.

According to some aspects of the invention, a use of a non-Tetranychid arthropod species comprises an application of individuals of a non-Tetranychid arthropod species on a target crop, preferably an immobilized non-Tetranychid arthropod species, such as a non-phytophagous prey, preferably an Astigmatid species, most preferably an immobilized Astigmatid species, such as an immobilized Astigmatid species, in particular a Carpoglyhus species, having immobilized life stages comprising immobilized eggs (for example a mixture of dead life stages, including dead eggs).

In a further embodiment, a mixture of eggs and mobile stages of the non-tetranychid prey is applied to a crop plant to be inhabited with Phytoseiulus predatory mites. The purpose of applying the prey directly on the plant is to support a population of P. persimilis or other natural enemy to be established on the plant when Tetranychid prey (the natural host of Phytoseiulus predatory species) is scarce. According to specific embodiments, devices for releasing the mobile stages of the prey, as disclosed in the application, are used.

The term “rearing composition” as used herein generally refers to a composition suitable for breeding, bringing up, raising, upbringing or propagating a mite species by sexual reproduction. Such a rearing composition and process is performed under artificial conditions, such as in a production (e.g. mass production) setup or system or container and preferably not in the natural growth habitat of the mite species. For example, in the context of the present invention, the rearing composition of P. persimilis may comprise Astigmatid mite species, preferably immobilized Astigmatic mite as a factitious host species for the P. persimilis predators. A rearing composition comprises a rearing population of the mite species, in particular the Phytoseiulus species. A rearing population may comprise sexually mature adults from both sexes, and/or individuals of both sexes of other life stages, e.g. eggs, larvae and/or nymphs, which can mature to sexually mature adults. Alternatively, the rearing population may comprise one or more fertilized females. In essence, a rearing population is capable of increasing the number of its individuals by means of sexual reproduction. More specifically, the term “rearing composition” refers to a composition suitable for the commercial rearing of mites. It is herein acknowledged that mass rearing systems for predatory mites heavily depend on the availability of suitable prey for the predators. Therefore, there is a continuing need to improve rearing systems of both predatory mites and mites suitable as rearing prey. To solve this problem, the present invention provides a composition or system specifically adapted for effectively and efficiently rearing mite species of the genus Phytoseiulus, especially Phytoseiulus persimilis with the unique genomic markers, a highly important predatory mite used for crop pest (spider mites) biological control. For the first time, whole genome information and genomic Single Nucleotide Polymorphism (SNPs) is provided for Phytoseiulus persimilis and especially Phytoseiulus persimilis shown to complete its life cycle and reproduce, i.e. for at least 2 generations, being reared on Astigmatid mite species.

The term “carrier” refers hereafter to an inactive or inert substance or particles or vehicle. In a preferred embodiment the rearing composition of the present invention comprises a carrier for the individuals of the mite species. The carrier can be any solid material which is suitable to provide a carrier surface to the mite individuals. Examples of suitable carriers are plant materials such as bran (e.g. wheat), sawdust (e.g. fine sawdust), corn cob grits, vermiculite, Poaceae husks, such as millet husks, or rice husks, etc. According to further aspects of the invention, a carrier material may include sawdust, wheat bran, buckwheat husks, rice husks or millet husks, or comprising a mixture thereof, preferably a carrier having elements comprising mite shelters and/or comprising surfaces to which mites can adhere.

The term “Phytoseiulus” as used hereafter refers to a genus of mites in the Phytoseiidae family. This genus of predatory mites is most frequently used to control two-spotted spider mites in greenhouses and outdoor crops. It is within the scope of the present invention that the genus Phytoseiulus contains the following species: Phytoseiulus fragariae, Phytoseiulus longipes, Phytoseiulus macropilis, Phytoseiulus persimilis, Phytoseiulus robertsi and Mesoseiulus longipes (e.g. see https://www.benemite.com/mlongipes.htm). The Phytoseiulus predatory mites are known as specialists for spider mites (mites of the family Tetranychidae) which are phytophagous mites.

The term “Phytoseiulus persimilis” or “P. persimilis” as used hereafter refers to a predatory mite population comprising the Phytoseiulus persimilis (P. persimilis). Phytoseiulus is a genus of mites in the Phytoseiidae family. This predatory mite is the mite predator most frequently used to control two-spotted spider mites in greenhouses and outdoor crops grown in mild environments.

P. persimilis is generally used for spider mite control and management. They are voracious predators of most of the spider mite pests (Tetranychus spp). Some of the species they impact include: the two-spotted mite Tetranychus urticae, the carmine red mite T. cinnabarinus, and the Pacific mite T. pacificus. Unlike Neoseiulus calfornicus (Order: Mesostigmata, Family: Phytoseiidae, Subfamily: Amblyseiinae) which may not eat for relatively long periods, Phytoseiulus persimilis must have fresh feed. Furthermore, according to existing knowledge Phytoseiulus persimilis is not flexible by its diet as other available predatory mite species for spider mite control, since it is known to only feed upon specific Tetranychus species, but not all of them.

The present invention discloses for the first time genomic sequences, haplotype and SNPs for P. persimilis populations successfully developed and reproduced on non-Tetranychid arthropod prey, in particular selected from the order Astigmata and from the immobilized Phytoseiidae. On the basis of the surprising finding that Phytoseiulus species, contrary to the general conviction in the art, can develop and reproduce on non-Tetranychid arthropod prey, new genetic markers are provided herein to identify and select for the desirable trait. Such unique and novel SNPs provide genetic tools that previously were not known and available for production, selection and for much more effective rearing and breeding of P. persimilis on the alternative diet compared to rearing P. persimilis on its conventional diet, which consists of phytophagous mites.

The term “factitious host” generally refers hereafter to an unnatural host or host other than the target host for the predatory mite; one that biocontrol practitioners may more readily rear under laboratory conditions compared to the natural host. In the context of the present invention, factitious host or prey refers to organisms unlikely to be attacked by a natural enemy or predatory mite in its natural habitat, but that is artificially used to support the natural enemy's development and/or reproduction. Usually it is a species that is easier and less expensive to rear. Examples within the scope of the present invention include storage mites (such as Astigmatid mites) for predatory mites (such as Phytoseiulus mite species), mite eggs for predatory insects and mites. According to further aspects, the term factitious host is used when a biological control agent is forced to feed on an insect or mite that it would not feed upon in nature. This can allow higher production levels. The present invention shows for the first time that species of commercially available Phytoseiulus mites can be mass reared using Astigmatid mites (Acari: Astigmata) as factitious prey.

The term “juvenile mite” or “juvenile mites” refers hereafter to mite developmental life stages or mite developmental phases or instar including egg, larva, nymphs e.g. protonymph and deutonymph (third instar) individuals.

The term “individual” or “individuals” or “mite individuals” refers in the context of the present invention to mite developmental stages including, but not limited to eggs, juvenile mite stages such as larva, protonymph and deutonymph (third instar) and adult individuals.

The term “mobile stages” refers hereafter to mite developmental stages including larva, protonymph, deutonymph (third instar) and adult stages.

The term “immobilized” used hereafter generally means that the non-Tetranychid arthropod prey individuals, preferably Astigmatid individuals, have been subjected to an immobilization treatment. An immobilization treatment should be construed to mean a treatment which impairs the motility that a prey individual has in any of its life stages (including immobile stages, i.e. eggs and any mobile developmental stage). Motility being the capability of moving spontaneously and independently. As the skilled person is aware of, life stages of mites which are motile are larvae, nymphs and adults. Thus treatments that impair the motility of any of these stages should be considered to be an immobilization treatment. In addition, treatments that prevent individuals to develop from a non-motile life stage, such as from the egg stage, to a motile life stage, should also be considered an immobilization treatment. According to a preferred embodiment, the immobilized mite individuals comprise eggs, larvae, nymphs or adults, preferably live stages comprising eggs, most preferably eggs combined with juvenile live stages. According to a further preferred embodiment, the prey individuals are permanently immobilized. A treatment rendering the prey individuals, preferably Astigmatid mites, “non-viable” (i.e causing death) may be considered a permanently immobilizing treatment. According to some embodiments of the present invention, the immobilized, preferably non-viable mite individuals are produced by or exposed to a treatment including, but not limited to, thermal treatment, such as freezing, freeze-drying, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof. WO2013/103294 further discloses immobilized Astigmatid mites and methods for obtaining them. As the non-tetranychod prey, non-hatching eggs of Astigmatid mites (e.g. immobilized by freezing or by radiation treatment), more preferably in combination with immobilized, in particular non-viable, juvenile Astigmatid live stages are most preferred in the context of the present invention.

The term “non-viable” used hereafter generally means not capable of living, growing, developing, or functioning. According to main aspects of the present invention it refers to dead or not alive or non-living or immobilized mites (i.e. any mite developmental stage or phase) or mite eggs. In a specific embodiment of the present invention, non-viable Astigmata mites and/or eggs are used as a prey for predatory mites of the Phytoseiulus genus.

According to a specific embodiment, the composition of the present invention comprises C. lactis eggs and/or mites and/or larvae or nymphs, immobilized by freezing, used as a prey or a food source for predatory mites of the Phytoseiulus genus. According to an aspect of the invention, the eggs mites and/or larvae may be non-viable or dead.

The term “Astigmatid” or “Astigmata” or “Astigmatic mites” or “Astigmatina” as used herein refers to mites order within the Subclass: Acari. The Astigmatina are a “cohort” of mites. Astigmatina belongs to the Sarcoptiformes, which contains the “biting” Acariformes. The Astigmata order contains superfamilies with over thousands of genera. Non limiting examples of such superfamilies and families, within the scope of the present invention may include:
Suborder: Acaridia
Superfamilies:
Schizoglyphoidea: examples of families include: Schizoglyphidae
Histiostomatoidea: examples of families include: Histiostomatidae, Guanolichidae
Canestrinioidea: examples of families include: Chetochelacaridae, Lophonotacaridae, Canestriniidae, Heterocoptidae
Hemisarcoptoidea: examples of families include: Chaetodactylidae, Hyadesiidae, Carpoglyphidae, Algophagidae, Hemisarcoptidae, Winterschmidtiidae
Glycyphagoidea: examples of families include: Euglycyphagidae, Chortoglyphidae, Pedetropodidae, Echimyopodidae, Aeroglyphidae, Rosensteiniidae, Glycyphagidae
Acaroidea: examples of families include: Sapracaridae, Suidasiidae, Lardoglyphidae, Glycacaridae, Gaudiellidae
Acaridae: examples of families include: Hypoderoidea, Hypoderidae
Suborder: Psoroptidia
Superfamilies:
Pterolichoidea: examples of families include: Oconnoriidae, Ptiloxenidae
Pterolichidae: examples of families include: Cheylabididae, Ochrolichidae, Gabuciniidae, Falculiferidae, Eustathiidae, Crypturoptidae, Thoracosathesidae, Rectijanuidae, Ascouracaridae, Syringobiidae, Kiwilichidae, Kramerellidae
Freyanoidea: examples of families include: Freyanidae, Vexillariidae, Caudiferidae
Analgoidea: examples of families include: Heteropsoridae, Analgidae, Xolalgidae, Avenzoariidae, Pteronyssidae, Proctophyllodidae, Psoroptoididae, Trouessartiidae, Alloptidae, Thysanocercidae, Dermationidae, Epidermoptidae, Apionacaridae, Dermoglyphidae, Laminosioptidae, Knemidokoptidae, Cytoditidae
Pyroglyphoidea: examples of families include: Pyroglyphidae, Turbinoptidae
Psoroptoidea: examples of families include: Psoroptidae, Galagalgidae, Lobalgidae, Myocoptidae, Rhyncoptidae, Audycoptidae, Listrophoridae, Chirodiscidae, Atopomelidae, Chirorhynchobiidae, Gastronyssidae, Lemurnyssidae, Pneumocoptidae, Sarcoptidae.

The claims further present Astigmatid species suitable as the non-Tetranychid arthropod prey in embodiments of the different aspects of the invention. According to many embodiments of the different aspects of the invention, selection of a non-Tetranychid arthropod prey from an Astigmatid species is most preferred. When used as a food source for the Phytoseiulus species, Astigmatid individuals are most preferably used in immobilized form, in particular in an immobilized form having immobilized life stages comprising immobilized (non-hatching) eggs. Immobilization by freezing is in particular suitable and is the most preferred method of immobilization for Astigmatid individuals. Immobilization by irradiation treatment is an alternative highly favorable immobilization method.

A preferable Astigmatid mite species used by the biological control system of the present invention as a factitious host population for the Phytoseiulus predatory mite, e.g. P. persimilis, is a mite species of the Carpoglyphidae family, more preferably Carpoglyphus lactis (C. lactis).

Carpoglyphidae is a mite family in the order Astigmatina, containing four genera: Carpoglyphus, Coproglyphus, Dichotomiopus and Pullea.

Carpoglyphus lactis), which is most preferably used by the present invention as a diet for rearing P. persimilis, belongs to the Carpoglyphus genus. Carpoglyphus lactis is acknowledged herein as a stored product mite, infesting saccharide-rich stored commodities including dried fruits, wine, beer, milk products, jams and honey. Since C. lactis is capable of feeding on stored products, it is highly desirable and cost effective to raise P. persimilis on this mite species, as shown for the first time by the present invention. When used as a food source for the Phytoseiulus species, Carpoglyphus lactis individuals are most preferably used in an immobilized form, in particular having immobilized life stages comprising immobilized (non-hatching) eggs (and/or immobilized mites). Immobilization by freezing is in particular suitable and the most preferred method of immobilization for Carpoglyphus lactis.

In a further embodiment of the different aspects of the present invention, the Phytoseiulus predatory mite, e.g. P. persimilis, can complete its life cycle and reproduce when feeding on immobilized, in particular non-viable mites and/or eggs of the species Carpoglyphus lactis and/or Dermatophagoides farinae both belonging to the Astigmata order.

The term “phenotype” refers hereafter to the composite of the organism's observable characteristics or traits, including its morphology or physical form and structure; its developmental processes; its biochemical and physiological properties; its behavior, and the products of behavior (diet or food source). An organism's phenotype may result from two basic factors: the expression of an organism's genetic code, or its genotype or genomic composition, and the influence of environmental factors, which may interact, further affecting phenotype. It is within the scope of the current invention that an improved development and reproduction on a non-Tetranychid arthropod prey is a phenotype or phenotypic characteristic or trait of the predatory Phytoseiulus mite individuals of the invention. As used herein, the terms “phenotype,” “phenotypic trait” or “trait” refer to one or more traits and/or manifestations of an organism. The phenotype can be a manifestation that is observable to the naked eye, or by any other means of evaluation known in the art, e.g., microscopy, biochemical analysis, behavior such as feeding or diet restrictions or capabilities, or an electromechanical assay. In some cases, a phenotype or trait is directly controlled by a single gene or genetic locus, i.e., a “single gene trait.” In other cases, a phenotype or trait is the result of several genes.

The term “trait” refers hereafter to characteristic or phenotype. A phenotypic trait may refer to the appearance or other detectable characteristic of an individual, resulting from the interaction of its genome, proteome and/or metabolome with the environment. For example, in the context of the present invention improved reproduction or an increased reproduction rate as described herein is a phenotypical trait characterizing the predatory mites of the present invention. According to a further embodiment of the present invention, a trait may also arise from interaction between the mite and its associated microorganisms. A trait may be inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment. A dominant trait results in a complete phenotypic manifestation at heterozygous or homozygous state; conventionally, a recessive trait manifests itself only when present at homozygous state. A trait may be associated, co-segregated, in linkage or in coupling phase with a nucleic acid sequence of a gene or one or more genomic loci, or a genetic marker such as Single Nucleotide Polymorphism (SNP), an inversion, a deletion or an indel.

The term “genetic linkage” is understood within the scope of the invention to refer to an association of characters in inheritance due to location of genes in proximity on the same chromosome, measured by percent recombination between loci (centiorgan, cM).

As used herein, the term “linkage” refers to the degree with which one marker locus is associated with another marker locus or some other locus (for example, improved rearing on a non-Tetranychid arthropod prey locus). The linkage relationship between a molecular marker and a phenotype may be given as a “probability” or “adjusted probability.” Linkage can be expressed as a desired limit or range. For example, in some embodiments, any marker is linked (genetically and physically) to any other marker when the markers are separated by less than about 50, 40, 30, 25, 20, or 15 map units (or cM). For example, embodiments of the invention herein, provide for marker loci closely linked to chromosomal regions comprising a nucleotide sequence of any one of SEQ ID NOs 1-91, associated with the trait of improved rearing (development and reproduction) on a non-Tetranychid arthropod prey.

A centimorgan (“cM”) is a unit of measure of recombination frequency. One cM is equal to a 1% chance that a marker at one genetic locus will be separated from a marker at a second locus due to crossing over in a single generation.

As used herein, the term “population” refers to a plurality of individuals. According to some embodiments the term includes a genetically heterogeneous collection of mites sharing a common genetic derivation or to a line.

In the context of the current invention, two distinct Phytoseiulus species lines, e.g. P. persimilis populations are herein disclosed. A line of populations, designated as P+ in which at least 5% of said P. persimilis individuals comprise within their genome at least one Single Nucleotide Polymorphism (SNP) marker selected from:
a. SNP at position 34739 of SEQ ID NO:84 or a variant, portion or complementary sequence thereof,
b. at least one SNP at a position selected from 105452, 109775, 115020, 115073, 125940, 131548 and 131555, of SEQ ID NO:85 or a variant, portion or complementary sequence thereof,
c. at least one SNP at a position selected from 99333, 99595, 99605, 115325 and 115354, of SEQ ID NO:86 or a variant, portion or complementary sequence thereof,
d. at least one SNP at a position selected from 4508, 11697, 12229, 13135, 32979, 38081, 38220, of SEQ ID NO:87 or a variant, portion or complementary sequence thereof,
e. at least one SNP at a position selected from 30913, 34950, 38409, 38458, 42427, 42604 and 43158 of SEQ ID NO:88 or a variant, portion or complementary sequence thereof,
f. SNP at position 1032 of SEQ ID NO:89 or a variant, portion or complementary sequence thereof,
g. SNP at position 173977 and/or SNP at position 228195 of SEQ ID NO:90 or a variant, portion or complementary sequence thereof, and
h. at least one SNP at a position selected from 857, 15689, 40135 and 49492 of SEQ ID NO:91 or a variant, portion or complementary sequence thereof.

In the context of the present invention, a P+ type population include “Alternating” population and “continuous” application.

This line of populations is reared and selected on a non-Tetranychid arthropod prey, preferably on an immobilized non-Tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs. In some aspects of the invention, the P+ population is characterized by improved reproduction on the non-Tetranychid arthropod prey, defined herein by parameters such as daily reproduction rate, daily oviposition rate, female and/or juvenile survival rate and percentage of female individuals capable of reproducing on the non-Tetranychid arthropod prey. This phenotypic trait is as compared to a population of P. persimilis individuals characterized by less than 5% individuals comprising within their genome said at least one SNP marker (P− population or non-P+ population).

Such a P+ type population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

The second line of populations (designated non-P+, or P−) is a population in which less than 5% of the individuals comprising within their genome said at least one SNP marker selected from:
a. SNP at position 34739 of SEQ ID NO:84 or a variant, portion or complementary sequence thereof,
b. at least one SNP at a position selected from 105452, 109775, 115020, 115073, 125940, 131548 and 131555, of SEQ ID NO:85 or a variant, portion or complementary sequence thereof,
c. at least one SNP at a position selected from 99333, 99595, 99605, 115325 and 115354, of SEQ ID NO:86 or a variant, portion or complementary sequence thereof,
d. at least one SNP at a position selected from 4508, 11697, 12229, 13135, 32979, 38081, 38220, of SEQ ID NO:87 or a variant, portion or complementary sequence thereof,
e. at least one SNP at a position selected from 30913, 34950, 38409, 38458, 42427, 42604 and 43158 of SEQ ID NO:88 or a variant, portion or complementary sequence thereof,
f. SNP at position 1032 of SEQ ID NO:89 or a variant, portion or complementary sequence thereof,
g. SNP at position 173977 and/or SNP at position 228195 of SEQ ID NO:90 or a variant, portion or complementary sequence thereof, and
h. at least one SNP at a position selected from 857, 15689, 40135 and 49492 of SEQ ID NO:91 or a variant, portion or complementary sequence thereof.

The P. persimilis P− line is continuously reared on its natural host, namely Tetranychid arthropod prey, or spider mites as the sole food source. In further aspects, the P. persimilis P− line is absent of the improved rearing properties on a non-Tetranychid arthropod as a food source, preferably on an immobilized non-Tetranychid arthropod prey, such as on an Astigmatid prey, most preferably on an immobilized Astigmatid prey. The P. persimilis P− line is further characterized by a daily reproduction rate (λ) equal or lower than 1.06.

The P− population is also referred to as wild type or the traditionally reared or commercially or naturally available Phytoseiulus or P. persimilis population, or conventionally reared population or non-selected population or control population or a Phytoseiulus population.

The term “contig” refers hereafter to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequence and to overlapping physical segments (fragments) contained in clones depending on the context.

The term “scaffold” generally refers herein to a portion of the genome sequence reconstructed from end-sequenced whole-genome shotgun clones. Scaffolds are composed of contigs and gaps. A contig is a contiguous length of genomic sequence in which the order of bases is known to a high confidence level. Gaps occur where reads from the two sequenced ends of at least one fragment overlap with other reads in two different contigs (as long as the arrangement is otherwise consistent with the contigs being adjacent). Since the lengths of the fragments are roughly known, the number of bases between contigs can be estimated.

As used herein, the phrase “genetic marker” or “molecular marker” or “genomic marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. A marker may be, but is not limited to, an allele, a gene or a haplotype. Genetic markers or molecular markers include, for example, Single Nucleotide Polymorphisms (SNPs), indels (i.e. insertions deletions), Simple Sequence Repeats (SSRs), Restriction Fragment Length Polymorphisms (RFLPs), Random Amplified Polymorphic DNAs (RAFDs), Cleaved Amplified Polymorphic Sequence (CAPS) markers, Diversity Arrays Technology (DArT) markers, and Amplified Fragment Length Polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

A genetic marker can be physically located in a position on a chromosome that is within or outside of the genetic locus with which it is associated (i.e., is intragenic or extragenic, respectively).

According to A genomic marker may be in a heterozygous state or in a homozygous state within the genome.

As used herein, the terms “marker” and “genetic marker” are used interchangeably to refer to a nucleotide and/or a nucleotide sequence that has been associated with a phenotype, trait or trait form. In some embodiments, a marker may be associated with an allele or alleles of interest and may be indicative of the presence or absence of the allele or alleles of interest in a cell or organism. A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art.

Markers corresponding to genetic polymorphisms between members of a population can be detected by methods well-established in the art. These include, e.g., nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASF1), detection of amplified variable sequences of the organism genome, detection of self-sustained sequence replication, detection of simple sequence repeats (SSRs), detection of single nucleotide polymorphisms (SNPs), and/or detection of amplified fragment length polymorphisms (AFLPs).

A “marker allele,” also described as an “allele of a marker locus,” can refer to one of a plurality of polymorphic nucleotide sequences found at a marker locus in a population that is polymorphic for the marker locus.

“Marker-assisted selection” (MAS) is a process by which phenotypes are selected based on marker genotypes. In some embodiments, marker genotypes are used to identify individuals that will be selected for a breeding program. In some embodiments, marker genotypes are used to identify individuals that will not be selected for a breeding program (i.e., counter-selected organisms or individuals), allowing them to be removed from the breeding population.

As used herein, the terms “marker locus” and “marker loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the terms “marker probe” and “probe” refer to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides may be used for nucleic acid hybridization. Alternatively, in some aspects, a marker probe refers to a probe of any type that is able to distinguish (i.e., genotype) the particular allele that is present at a marker locus.

As used herein, the terms “molecular marker” or “genetic marker” may be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Any suitable marker detection technology may be used to identify such a hybridization marker, e.g., SNP technology is used in the examples provided herein.

A non-naturally occurring P. persimilis line or population is any P. persimilis genome of a line or population that does not naturally exist in nature. This non-naturally occurring P. persimilis contain (or at least 5% of the population contain) the haplotype/markers of the present invention (see Tables 1-7).

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence are known in the art, and include, for example, cloning and restriction of appropriate sequences and direct chemical synthesis. Primers diagnostic (i.e. able to identify or select based on presence of the unique P. persimilis genomic regions associate with improved development and reproduction on a non-Tetranychid arthropod prey) can be created to any favorable SNP as described in any one of Tables 1-2. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non specific/adventitious binding. Conditions that affect hybridization, and that select against non-specific binding are known in the art.

The term “homology” is meant DNA sequences that are similar. For example, a “region of homology to a genomic region” is a region of DNA that has a similar sequence to a given “genomic region” in the cell or organism genome. A region of homology can be of any length that is sufficient to promote homologous recombination at the cleaved target site, such that the region of homology has sufficient homology to undergo homologous recombination with the corresponding genomic region. “Sufficient homology” indicates that two polynucleotide sequences have sufficient structural similarity to act as substrates for a homologous recombination reaction or have the same function. The structural similarity includes overall length of each polynucleotide fragment, as well as the sequence similarity of the polynucleotides.

“Sequence similarity” can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range. The amount of homology can also be described by percent sequence identity over the full aligned length of the two polynucleotides which includes percent sequence identity (or similarity) of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology-Hybridization with Nucleic Acid Probes, (Elsevier, New York).

The structural similarity between a given genomic region and the corresponding region of homology (e.g. found on the donor DNA) can be of any degree of sequence identity that allows for homologous recombination to occur. For example, the amount of homology or sequence identity shared by the “region of homology” a corresponding DNA (e.g. of the donor DNA) and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

As used herein, “homologous recombination” includes the exchange of DNA fragments between two DNA molecules at the sites of homology.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

As used herein, a “genomic region” is a segment of a chromosome in the genome of a cell that is present on either side of the genomic marker (SNP). Such genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases and it may have at its two ends primer sequences for amplifying the genomic region. In further aspects, the term “genomic region” used in reference to particular loci/SNP and/or alleles, refers to a chromosomal interval delimited by and encompassing the stated loci/SNP/alleles.

As used herein, the term “homozygous” refers to a genetic condition or configuration existing when two identical or like alleles reside at a specific locus, but are positioned individually on corresponding pairs of homologous chromosomes in the cell of a diploid organism. In other words, it means a genetic status wherein identical alleles reside at corresponding loci on homologous chromosomes.

Conversely, as used herein, the term “heterozygous” means a genetic condition or configuration existing when two different or unlike alleles reside at a specific locus, but are positioned individually on corresponding pairs of homologous chromosomes in the cell of a diploid organism. It further means a genetic status wherein different alleles reside at corresponding loci on homologous chromosomes.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing. It is also within the scope that the term “genotype” refers to the genetic constitution of a cell or organism. An individual's genotype includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest. Thus, in some embodiments a genotype comprises a summary of one or more alleles present within an individual at one or more genetic loci. In some embodiments, a genotype is expressed in terms of a haplotype.

As used herein, the term “germplasm” refers to the totality of the genotypes of a population or other group of individuals (e.g., a species).

According to further aspects, the term “germplasm” refers to genetic material of or from an individual (e.g. a mite), a group of individuals (e.g. a mite population or line, species, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture.

The term “population” or “rearing population”, as used herein, refers to a population that can increase their number by sexual reproduction. A rearing population can comprise alive sexually mature individuals of both sexes and/or other stages of life, for example, eggs and/or nymph from both sexes that can mature into sexually mature adults. Alternatively, a rearing population can comprise one or more fertilized females.

The term “pest”, as used herein, refers to plant pest, and is understood to include any species, strain or biotype of plant, animal or pathogenic agent injurious for plant or plant products. The pests that can be controlled by the method of the invention are animals that are prey for the predator P. persimilis. In a particular embodiment, the pest is selected from the group consisting of Tetranychidae mites, Thrips, whiteflies, aphids and moths.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment. It is also within the scope of the present invention that the term “haplotype” (haploid genotype) refers to a group of alleles in an organism that are inherited together from a single parent. In other aspects of the invention it is used to mean a set of linked DNA sequences, such as Single-Nucleotide Polymorphism (SNP) alleles, indels, that tend to occur together (i.e., that are associated statistically). According to further aspects of the invention, it refers to a collection of specific mutations within a given genetic segment.

The term haplotype further means a set of DNA variations, or polymorphisms, that tend to be inherited together. A haplotype can refer to a combination of alleles or to a set of Single Nucleotide Polymorphisms (SNPs) or other genetic variations found on the same chromosome.

According to some embodiments, the P. persimilis haplotype of the present invention is associated with the characteristic of improved rearing on a non-Tetranychid arthropod prey as compared to P. persimilis lacking the haplotype, which characterizes naturally occurring P. persimilis populations and/or P. persimilis predatory mites traditionally or conventionally reared on Tetranychid prey species (e.g. spider mites).

The term “allele(s)” used herein means any of one or more alternative or variant forms of a gene or a genetic unit at a particular locus, all of which alleles relate to one trait or characteristic at a specific locus. It further means one of two or more different nucleotides or nucleotide sequences that occur at a specific locus. In a diploid cell of an organism, alleles of a given gene are located at a specific location, or locus (loci in plural) on a chromosome. One allele is present on each chromosome of the pair of homologous chromosomes. Such alternative or variant forms of alleles may be the result of single nucleotide polymorphisms, insertions, indels, inversions, translocations or deletions, or the consequence of gene regulation caused by, for example, chemical or structural modification, transcription regulation or post-translational modification/regulation. An allele associated with a qualitative trait may comprise alternative or variant forms of various genetic units including those that are identical or associated with a single gene or multiple genes or their products or even a gene disrupting or controlled by a genetic factor contributing to the phenotype represented by the locus.

As used herein, the terms “desired allele”, “favorable allele” and “allele of interest” are used interchangeably to refer to an allele associated with a desired trait (e.g. improved rearing properties on a non-Tetranychid arthropod prey). As used herein, the term “locus” means a specific place or places or region or a site on a chromosome where for example a gene or genetic marker element or factor is found. In specific embodiments, such a genetic element is contributing to a trait A “locus” is a position on a chromosome where a gene or marker or allele is located. In some embodiments, a locus may encompass one or more nucleotides.

The term “cultivar” or “strain” used herein means a group of similar mite individuals that by features (e.g. genetic features and performance) can be identified from other strains within the same species e.g. identified from the natural existing strain. A cultivated mite or mite population or line refers hereafter to a modified mite individual or plurality of individuals or a population, e.g. subjected to a breeding and selection processes and/or comprising genetic sequences that differentiate them from the natural or existing corresponding mite populations or individuals that were not subjected to the breeding and selection processes and to the artificial growth conditions.

The term “genetic background” used herein refers to any inbreeding group, including taxonomic subgroups such as subspecies, taxonomically subordinate to species and superordinate to a race or subrace and marked by a pre-determined profile of latent factors of hereditary traits.

As used herein, the term “breeding” and grammatical variants thereof, refer to any process that generates a progeny individual. Breeding can be sexual or asexual, or any combination thereof. Exemplary non-limiting types of breeding include crossing, introgressing, selfing, backcrossing, doubled haploid derivative generation, and combinations thereof.

The term “genetic determinant” as used herein refers to genetic determinants such as genes, alleles, genetic loci, QTLs or traits.

Introgression of a genetic determinant means the incorporation of genes, alleles, genetic loci, QTLs or traits into a line wherein essentially all of the desired morphological and physiological characteristics of the line are recovered, in addition to the genetically introgressed determinant. Such a process is often used in cultivar/line/species/variety development, in which one or a few genetic determinants are transferred to a desired genetic background, preferably by using backcrossing.

In further aspects of the present invention, the term introgression or breeding refers to the transmission of a desired allele or genomic region or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, where at least one of the donors has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, a marker associated with enhanced rearing on a non-Tetranychid arthropod prey (e.g. enhanced development and reproduction) may be introgressed from a donor into a recurrent parent that lacks this characteristic. The resulting offspring could then be repeatedly backcrossed and selected until the progeny possess the desired allele(s) in the recurrent parent background.

As used herein, the term “co-segregate” is understood within the scope of the invention to refer to the tendency for genes, traits and/or genetic markers or genetic variants to segregate, to be in coupling phase, or to be inherited together. Alternatively, two or more genes, gene alleles or genetic markers that are linked on the same chromosome are transmitted to the same daughter cell leading to the inheritance by the offspring of these genes or alleles together.

According to one embodiments, the genomic haplotype is associated or in coupling phase with the characteristic of improved rearing properties on a non-Tetranychid arthropod as a factitious host, preferably on an immobilized non-Tetranychid arthropod prey, such as on an Astigmatid prey, most preferably on an immobilized Astigmatid prey.

According to one aspect, the improved reproduction is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

The term “fungus reducing agent” or “fungal reducing agent” refers hereafter to chemical fungus reducing agents such as a natural or synthetic fungicide, or to a biological fungus reducing agent such as a population of a mite species producing antifungal exudates, or a population of mycophagous mites, in particular selected from the Astigmata, for example populations of living Carpoglyphus lactis or Lepidoglyphus destructor individuals. Such fungus reducing mite populations are disclosed in WO2013/103294.

It is within the scope of the current invention that the rearing composition as defined in any of the above is absent of or is lacking a fungus reducing agent. The claimed Phytoseiulus mite population of the present invention is characterized in that at least 5% of said P. persimilis individuals comprise within their genome at least one Single Nucleotide Polymorphism (SNP) marker selected from:
a. SNP at position 34739 of SEQ ID NO:84 or a variant, portion or complementary sequence thereof,
b. at least one SNP at a position selected from 105452, 109775, 115020, 115073, 125940, 131548 and 131555, of SEQ ID NO:85 or a variant, portion or complementary sequence thereof,
c. at least one SNP at a position selected from 99333, 99595, 99605, 115325 and 115354, of SEQ ID NO:86 or a variant, portion or complementary sequence thereof,
d. at least one SNP at a position selected from 4508, 11697, 12229, 13135, 32979, 38081, 38220, of SEQ ID NO:87 or a variant, portion or complementary sequence thereof,
e. at least one SNP at a position selected from 30913, 34950, 38409, 38458, 42427, 42604 and 43158 of SEQ ID NO:88 or a variant, portion or complementary sequence thereof,
f. SNP at position 1032 of SEQ ID NO:89 or a variant, portion or complementary sequence thereof,
g. SNP at position 173977 and/or SNP at position 228195 of SEQ ID NO:90 or a variant, portion or complementary sequence thereof, and
h. at least one SNP at a position selected from 857, 15689, 40135 and 49492 of SEQ ID NO:91 or a variant, portion or complementary sequence thereof.

In such a population at least 5% of the individuals are capable of completing their life cycle and reproducing for at least 2 generations when fed on immobilized Astigmata individuals including mites at any developmental stage and/or eggs. It is noted that non-viable Astigmata mite developmental stages are incapable of producing or secreting a fungus reducing agent.

Such a population may be further characterized by the following embodiments:

According to one embodiment, rearing by an alternative method for rearing P. persimilis and other mite species of the genus Phytoseiulus. The mite species of the genus Phytoseiulus, e.g. P. persimilis is shown to broaden its dietary range, and be reared on other preys, which are cheaper to produce and therefore much more desirable. The alternative prey mites are mostly Astigmatic mites that feed on stored products and are therefore significantly cheaper to produce.

According to a further embodiment, a system and method for using mites (especially dead or otherwise immobilized mites) of the species Carpoglyphus lactis (Cl) or other Astigmatic mite as an alternative food for mite species of the Phytoseiulus genus, such as Phytoseiulus persimilis, is provided.

According to a further embodiment, it is shown by the present invention that mite species Phytoseiulus persimilis comprising the genetic markers of the present invention can complete its life cycle and reproduce when feeding on dead mites belonging to the order Astigmata (within the Arachnida class).

It is further within the scope to develop a system for the production of mite species of the genus Phytoseiulus, e.g. Phytoseiulus persimilis, comprising the genetic markers of the present invention, on a diet comprising Astigmatic mites. The system is based on the following components:
1. The predator—specifically Phytoseiulus persimilis and more generally mites of the genus Phytoseiulus.
2. The prey—a mite species, possibly Carpoglyphus lactis, Glyciphagus domesticus, Lepidoglyphus destructor, Dermatophagoides farinae, Dermatophagoides pteronisinus or other Astigmatic mite, or other mite species such as Amblyseius swirskii.
3. The rearing system—the specific setup in which the mites are reared, comprising the rearing media, the way the prey mite is presented to the predator, the prey developmental stage and other factors.

The following rearing methods are within the scope of the present invention:
1. The predator is reared on a living mixture of prey mites.
2. The predator receives a mixture of immobilized prey mites by means of freezing or by other means such as irradiation.
3. A certain developmental stage of the prey mite is extracted from the prey mite's population, and then served alive or dead as food to the predator.

It is noted that in all of the above optional rearing methods, the prey mite could be either the above mentioned Astigmatic mites, or other species.

With respect to the final biological control product, the following is within the scope of the present invention:
1. A mixture which contains both the predator and the prey mites, or the predator and specific stages of the prey mites used for feeding the predator.
2. A further option is extracting only the predators, so that the final product contains only the predators.

According to one embodiment, the present invention provides a biological control composition comprising a cultivated rearing population of Phytoseiulus persimilis (P. persimilis) predatory mite individuals, and optionally a carrier material, wherein at least 5% of said P. persimilis individuals comprise within their genome at least one Single Nucleotide Polymorphism (SNP) marker selected from:
a. SNP at position 34739 of SEQ ID NO:84 or a variant, portion or complementary sequence thereof,
b. at least one SNP at a position selected from 105452, 109775, 115020, 115073, 125940, 131548 and 131555, of SEQ ID NO:85 or a variant, portion or complementary sequence thereof,
c. at least one SNP at a position selected from 99333, 99595, 99605, 115325 and 115354, of SEQ ID NO:86 or a variant, portion or complementary sequence thereof,
d. at least one SNP at a position selected from 4508, 11697, 12229, 13135, 32979, 38081, 38220, of SEQ ID NO:87 or a variant, portion or complementary sequence thereof,
e. at least one SNP at a position selected from 30913, 34950, 38409, 38458, 42427, 42604 and 43158 of SEQ ID NO:88 or a variant, portion or complementary sequence thereof,
f. SNP at position 1032 of SEQ ID NO:89 or a variant, portion or complementary sequence thereof,
g. SNP at position 173977 and/or SNP at position 228195 of SEQ ID NO:90 or a variant, portion or complementary sequence thereof, and at least one SNP at a position selected from 857, 15689, 40135 and 49492 of SEQ ID NO:91 or a variant, portion or complementary sequence thereof.

According to a further aspect, the present invention provides a predatory mite population comprising Phytoseiulus predatory individuals (preferably comprising any of the herein disclosed genetic markers or any combination thereof), wherein the population is characterized by a daily oviposition rate of at least 0.55, such as ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female while preying on non-Tetranychid arthropod prey. This aspect of the invention includes embodiments wherein the percentage of female individuals capable of reproduction on a non-Tetranychid arthropod prey is not specified (is unspecified).

According to yet a further aspect, the invention relates to a biological control composition wherein the composition comprises:
a. a predatory mite population comprising individuals of at least one mite species of the genus Phytoseiulus (preferably comprising any of the herein disclosed genetic markers or any combination thereof) capable of development and reproduction on a non-Tetranychid arthropod prey, preferably on an immobilized non-Tetranychid arthropod prey, such as on a non-phytophagous prey, preferably on an Astigmatid prey, most preferably on an immobilized Astigmatid prey, such as an immobilized Astigmatid prey, in particular a Carpoglyhus prey, having immobilized life stages comprising immobilized eggs; and
b. a prey mite population comprising individuals of a non-Tetranychid arthropod prey, preferably an immobilized non-Tetranychid arthropod prey, such as a non-phytophagous prey, preferably an Astigmatid prey, most preferably an immobilized Astigmatid prey, such as an immobilized Astigmatid prey, in particular a Carpoglyhus prey, having immobilized life stages comprising immobilized eggs, and
c. optionally a carrier, such as a carrier material selected from sawdust, wheat bran, buckwheat husks, rice husks or millet husks, or comprising a mixture thereof, preferably a carrier having carrier elements comprising mite shelters and/or comprising surfaces to which mites can adhere to. A non-Tetranychid arthropod prey according to the present invention is a prey selected from arthropods other than Tetranychids. The non-Tetranychid arthropod prey may be a non-phytophagous prey, preferably an Astigmatid prey. In the present invention most preferably, an immobilized Astigmatid prey is used as the non-Tetranychid arthropod prey, in particular an immobilized Astigmatid prey having immobilized life stages comprising immobilized eggs.

A further aspect of the invention relates to a biological control composition comprising Phytoseiulus predatory individuals (preferably comprising any of the herein disclosed genetic markers or any combination thereof), wherein the population is characterized by a daily oviposition rate of at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female while preying on non-Tetranychid arthropod prey, preferably on an immobilized non-Tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs.

According to a further aspect, the invention relates to a biological control composition comprising Phytoseiulus predatory individuals (preferably comprising any of the herein disclosed genetic markers or any combination thereof), wherein the population is characterized by improved reproduction on non-Tetranychid arthropod prey, preferably on an immobilized non-Tetranychid arthropod prey, such as on a non-phytophagous prey, preferably on an Astigmatid prey, most preferably on an immobilized Astigmatid prey, such as an immobilized Astigmatid prey, in particular a Carpoglyphus prey, having immobilized life stages comprising immobilized eggs. The improved reproduction on non-Tetranychid arthropod prey is as compared to a control Phytoseiulus predatory population of the same species which lacks the genetic markers, traditionally reared on the P. persimilis natural host (spider mites).

According to certain embodiments of the different aspects of the invention, female individuals (preferably comprising any of the herein disclosed genetic markers or any combination thereof) capable of development and reproduction on the non-Tetranychid arthropod prey, are female individuals capable of oviposition on non-Tetranychid arthropod prey, preferably on immobilized Astigmatid prey having immobilized life stages comprising immobilized eggs. As the skilled person will understand, oviposition capability relates to the capability to lay or produce eggs. Determining oviposition rates is within the ambit of the skill of the skilled person. The oviposition capability of the females preferably is determined after being fed for at least 4 days on the non-Tetranychid prey, such as after 5 days or after 6 days.

The daily oviposition rate of a predatory mite population (preferably comprising any of the herein disclosed genetic markers or any combination thereof) according to various aspects of the invention may be at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female.

In some embodiments, the daily oviposition rate is at least 1 egg per day per female, particularly at least 1.4 eggs/day/female, more particularly between 1.4-2 eggs/day/female. In main aspects of the present invention, a daily oviposition rate of at least 1 egg per day per female, particularly at least 1.4 eggs/day/female, more particularly between 1.4-2 eggs/day/female, is achieved when using the non-Tetranychid arthropod prey as the sole food source for the Phytoseiulus predatory individuals (preferably comprising any of the herein disclosed genetic markers or any combination thereof). According to further aspects of the present invention, a daily oviposition rate of at least 1 egg per day per female, particularly at least 1.4 eggs/day/female, more particularly between 1.4-2 eggs/day/female, is achieved when using the non-Tetranychid arthropod prey as a food source for the Phytoseiulus predatory individuals in alternation with spider mites diet.

Within the present invention the term “at least” in the context of numerical values is considered equivalent with the meaning of the mathematical sign “≥”. The skilled person will understand that, being an average value for (the female part of) the population, the oviposition rate or egg production rate may have a fractional value not corresponding to whole eggs. The skilled person will also understand that a mite population having a daily oviposition rate of at least 0.50 eggs/day/female is capable to produce 0.5 eggs/day/female or more. Thus defined differently, a predatory mite population having a daily oviposition rate of ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female is capable of producing respectively 0.55, 0.60, 0.65, 0.70, 0.75, 0.80, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00 eggs/day/female. Again, if more eggs then the indicated numbers are produced, the indicated number of eggs are produced.

The daily oviposition rate of a predatory mite population (preferably comprising any of the herein disclosed genetic markers or any combination thereof) according to other embodiments of the different aspects of the invention may be at least 0.50, such as ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or at least 2.00 eggs/day/female, when using the non-Tetranychid arthropod prey as the sole food source or alternatively when preying on the non-Tetranychid arthropod prey. Also in this case, defined differently, a predatory mite population having a daily oviposition rate of ≥0.55, ≥0.60, ≥0.65, ≥0.70, ≥0.75, ≥0.80, ≥0.90, ≥0.95, ≥1.00, ≥1.05, ≥1.10, ≥1.15, ≥1.20, ≥1.25, ≥1.30, ≥1.35, ≥1.40, ≥1.45, ≥1.50, ≥1.55, ≥1.60, ≥1.65, ≥1.70, ≥1.75, ≥1.80, ≥1.85, ≥1.90, ≥1.95, or ≥2.00 eggs/day/female is capable of producing respectively 0.55, 0.60, 0.65, 0.70, 0.75, 0.80, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00 eggs/day/female when using the non-Tetranychid arthropod prey as the sole food source. When using the non-Tetranychid prey as the sole food source no other food is presented to the individuals of the predatory mite population. As stated before, if more eggs then the indicated numbers are produced, the indicated number of eggs are produced.

Capability to reproduce on non-Tetranychid arthropod prey according to certain embodiments of different aspects of the invention most preferably includes the capability of completing a full ontogenetic cycle, when using the non-Tetranychid arthropod prey as the sole food source. Completion of the ontogenic cycle, as the skilled person will understand is the capability of individuals to develop from the earlies life stage to a subsequent earlies life stage in a second generation, viz. for predatory mites development from a (parent) egg to an (offspring) egg in a next generation or defined differently development from an egg to a sexually mature female individual producing a number of eggs. The skilled person will know and understand that for many predatory mite species, including Phytoseiulus species, copulation with a male individual is required for egg production in females. If a population is capable to complete the ontogentic cycle on a certain food source, it can in theory perpetually cycle through multiple generations on that food source.

Capability to develop and reproduce on non-Tetranychid arthropod prey according to certain embodiments of different aspects of the invention, is characterized by capability of female individuals to produce female offspring in a number of subsequent generations. The number of subsequent generations is at least 1, such as at least 2, such as in at least 3, 4, 5, 6, 7, 8, 9 at least 10 generations. The skilled person will understand that in case the number of subsequent generations, is at least 2, a full ontogenetic cycle is completed, as the female offspring of the female has produced (female) offspring. Thus the number of subsequent generations preferably is at least 2, such that at least one ontogentic cycle is completed.

Capability to develop and reproduce on non-Tetranychid arthropod prey according to certain embodiments of different aspects of the invention may also include a juvenile and/or female survival rate of at least 40%, on the non-Tetranychid prey used as the sole food source. As the skilled person will understand, the juvenile survival rate is the percentage of juvenile life stages that is capable of developing to the adult stage. Juvenile survival rates in the context of the present invention are determined as the percentage post embryonic (post-egg) stages that reach adulthood. The juvenile survival rate is determined on the non-Tetranychid prey, preferably an immobilized Astigmatid prey having immobilized life stages comprising immobilized eggs, used as the sole food source. Juvenile survival is determined over a period of between 3 to 7 days, such as over a period of 2, 3, 4, 5, 6, or 7 days, most preferably during a period of 3 days. The female survival rate is the rate of mature females that survive on the non-Tetranychid prey, preferably an immobilized Astigmatid prey having immobilized life stages comprising immobilized eggs, when used as the sole food source. Female survival is determined over a period of 7 days. At least 40% for the juvenile survival rate may be between 40% and 95%, such as 45%-90%, 50%-90%, 55-90%, 60%-90%, 65%-90%, 70%-90%, 75%-90%, 45%-85%, 50%-85%, 55-85%, 60%-85%, 65%-85%, 70%-85%, 75%-85%. At least 40% for the female survival rate may be at least 45%, at least 50%, at least 55%, at least 60%, at least 65%, at least 70%, at least 75%, at least 80%, at least 85%, at least 90%, at least 95%, at least 99%. At least 40% and all higher percentages mentioned, include substantially all and 100%.

In some embodiments, the juvenile and/or female survival rate is at least 60%, particularly at least 80% and up to 100%. In main aspects of the present invention, a juvenile and/or female survival rate of at least 60%, particularly at least 80% and up to 100%, is achieved when using the non-Tetranychid arthropod prey as the sole food source for the Phytoseiulus predatory individuals (preferably comprising any of the herein disclosed genetic markers or any combination thereof). According to further aspects of the present invention, a juvenile and/or female survival rate of at least 60%, particularly at least 80% and up to 100%, is achieved when using the non-Tetranychid arthropod prey as a food source for the Phytoseiulus predatory individuals in alternation with spider mites diet.

Capability to reproduce on non-Tetranychid arthropod prey according to certain embodiments of different aspects of the invention may also be characterized by a daily multiplication (or reproduction) rate λ in the range of about 1.10-1.40, such as 1.15-1.40, 1.20-1.40, 1.25-1.40, 1.30-1.40, or 1.10-1.35, 1.10-1.30, 1.10-1.25, 1.10-1.20. The skilled person will understand that for values for λ above 1.0 the population increases, thus there is reproduction. The skilled person will further understand that due to starvation in a population, also under the circumstance that values for λ for a given population are somewhat below 1.0, individuals in the population may be reproducing (to a level that is not compensating for the level of mortality due to starvation). The daily multiplication rate according to preferred embodiments relates to daily multiplication rates when using the non-Tetranychid arthropod prey as the sole food source. Although daily multiplication (or reproduction) rates above 1.0 have been observed by the inventors of the present invention for Phytoseiulus populations reared on spider mites and not comprising the at least one genetic marker of the present invention, these Phytoseiulus populations do not have daily multiplication (or reproduction) rates of 1.10 or above.

In some embodiments, the daily multiplication (or reproduction) rate λ is at least 1.15, particularly at least 1.2, more particularly 1.2-1.4. In main aspects of the present invention, a daily multiplication (or reproduction) rate λ of at least 1.15, particularly at least 1.2, more particularly 1.2-1.4, is achieved when using the non-Tetranychid arthropod prey as the sole food source for the Phytoseiulus predatory individuals (preferably comprising any of the herein disclosed genetic markers or any combination thereof). According to further aspects of the present invention, a daily multiplication (or reproduction) rate λ of at least 1.15, particularly at least 1.2, more particularly 1.2-1.4, is achieved when using the non-Tetranychid arthropod prey as a food source for the Phytoseiulus predatory individuals (preferably comprising any of the herein disclosed genetic markers or any combination thereof) in alternation with spider mites diet.

In general, within the context of the present invention, life stages parameters, such as oviposition rates and survival rates, completion of the ontogentic cycle and population growth rates of predatory mites may be determined at 22 degrees Celsius and 85% relative humidity, while food (the non-Tetranychid arthropod prey) is not limiting (presented ad libidum).

According to preferred embodiments of the predatory mite population of the invention (preferably comprising any of the herein disclosed genetic markers or any combination thereof), predatory individuals have predatory behavior towards individuals of a Tetranychid species. Preferably, female individuals have predatory behavior towards Tetranychid individuals. If in the population at least 10% of the female individuals is capable of development and reproduction a non-Tetranychid arthropod prey, most preferably this at least 10% of the female individuals exhibit predatory behavior towards individuals of a Tetranychid species. By maintaining predatory behavior towards individuals of a Tetranychid species, the predatory mite individuals can be used as biocontrol agents against the Tetranychid species on which they predate. According to preferred embodiments, the predatory behavior towards individuals of a Tetranychid species may result in a daily oviposition rate of at least 10, preferably at least 15, more preferably at least 19 eggs per female per 5 days.

According to some embodiments of the present invention, the above described predatory behavior towards individuals of a Tetranychid species is achieved when using the non-Tetranychid arthropod prey as the sole food source for the reared Phytoseiulus predatory individuals (preferably comprising any of the herein disclosed genetic markers or any combination thereof). According to further aspects of the present invention the above described predatory behavior towards individuals of a Tetranychid species, is achieved when using the non-Tetranychid arthropod prey as a food source for the reared Phytoseiulus predatory individuals (preferably comprising any of the herein disclosed genetic markers or any combination thereof) in alternation with spider mites diet.

According to one embodiment of the different aspects of the invention, the invention provides a rearing composition comprising: a predatory mite population (preferably comprising any of the herein disclosed genetic markers or any combination thereof) comprising at least one mite species of the genus Phytoseiulus, and a prey mite population comprising individuals of at least one mite species from the order Astigmata, wherein said predatory mite population is capable of oviposition for at least 2 generations, further wherein said Astigmata prey is selected from the group consisting of immobilized mites, preferably non-viable mites, non-hatching (immobilized) eggs, preferably non-viable eggs and a combination thereof.

It is within the scope of the present invention that the predatory mite (preferably comprising any of the herein disclosed genetic markers or any combination thereof) is capable of oviposition for at least 10 generations and preferably more, having the Astigmata individuals as a prey.

It is further within the scope that the predatory mite population (preferably comprising any one of the herein disclosed genetic markers or any combination thereof) exhibits an increased reproduction rate trait, in particular when using Astigmatid mites as food source, as compared to a control predatory mite population, of the same species, lacking the aforementioned trait.

It is further within the scope that the predatory mite population of the present invention (preferably comprising any one of the herein disclosed genetic markers or any combination thereof) exhibits a daily reproduction rate in the range of about 1.15-1.2, in particular when using Astigmatid mites as food source.

It is further within the scope of the present invention that the predatory mite population (preferably comprising any one of the herein disclosed genetic markers or any combination thereof) is characterized by a beige-white color, when said Phytoseiulus predatory mite is reared upon said Astigmata prey as a food source.

It is within the scope of the present invention that the predators (preferably comprising any one of the herein disclosed genetic markers or any combination thereof) would have a different appearance than that of the common product containing P. persimilis mites reared on spider mites (white mites in the case of the present invention instead of the usual orange).

According to a further embodiment, the present invention shows that a population of P. persimilis comprising any one of the herein disclosed genetic markers or any combination thereof, successfully developed and reproduced on dead Carpoglyphus lactis for at least six months (about 25 generations).

It is emphasized that P. persimilis (preferably comprising any one of the herein disclosed genetic markers or any combination thereof) is herein surprisingly reported to complete its life cycle and reproduce on either non-phytophagous prey (prey that doesn't require to feed on living plants), or prey that doesn't consume phytophagous mites.

The present invention provides a mite composition which contains a Phytoseiulus persimilis rearing mite population (preferably comprising any one of the herein disclosed genetic markers or any combination thereof), and a factitious host mite population comprising at least one species from the order Astigmata or from the family Phytoseiidae.

The present invention solves the serious problem of rearing the main spider-mite controlling predator, Phytoseiulus persimilis, by rearing it in a cost effective and efficient way on a non-phytophagous alternative diet.

Accordingly, the invention provides a mite composition comprising: a rearing population of mite species of the genus Phytoseiulus, for example Phytoseiulus persimilis predatory mite species (preferably comprising any one of the herein disclosed genetic markers or any combination thereof), a population of at least one species from the order Astigmata or from the family Phytoseiidae, and optionally a carrier.

According to one embodiment of the different aspects, the present invention provides a rearing composition comprising: predatory mite population comprising at least one mite species of the genus Phytoseiulus (preferably comprising any one of the herein disclosed genetic markers or any combination thereof), and a prey mite population comprising at least one species from the order Astigmata.

According to a further embodiment of the different aspects, the present invention provides a method for rearing predatory mite population comprising at least one mite species of the genus Phytoseiulus (preferably comprising any one of the herein disclosed genetic markers or any combination thereof), the method comprising: (a) providing a composition comprising a predatory mite population comprising at least one mite species of the genus Phytoseiulus, and a prey mite population comprising at least one species from the order Astigmata; and (b) allowing individuals of the predatory mite population to prey on individuals of the Astigmatid population.

According to a further embodiment of the different aspects, the present invention provides a rearing composition comprising: predatory mite population comprising at least one mite species of the genus Phytoseiulus (preferably comprising any one of the herein disclosed genetic markers or any combination thereof), and a prey mite population comprising at least one species from the Phytoseiidae family.

According to a further embodiment of the different aspects, the present invention provides a method for rearing predatory mite population comprising at least one mite species of the genus Phytoseiulus (preferably comprising any one of the herein disclosed genetic markers or any combination thereof), the method comprising: (a) providing a composition comprising: predatory mite population comprising at least one mite species of the genus Phytoseiulus, and a prey mite population comprising at least one species from the Phytoseiidae family; and (b) allowing individuals of the predatory mite population to prey on individuals of the Phytoseiidae family population.

In some embodiments of the different aspects, the prey population i.e. species from the order Astigmata or species from the Phytoseiidae family, is immobilized and/or not alive.

It is further within the scope of the different aspects that the Phytoseiulus persimilis predatory mite (preferably comprising any one of the herein disclosed genetic markers or any combination thereof) is capable of reproducing for at least 2 generations, preferably at least 10 generations, more preferably for at least 15 generations or more generations, feeding on the aforementioned Astigmata or Phytoseiid population, in particular an immobilized population.

The composition of the present invention provides a considerable number of advantages over previous combinations. In one aspect, the food material used to feed the prey during predator production will no longer be plants or phytophagous mites, but mites that live upon stored products, therefore providing a substantial cost saving.

In another aspect, the present invention provides a rearing composition comprising: predatory mite population comprising at least one mite species of the genus Phytoseiulus (preferably comprising any one of the herein disclosed genetic markers or any combination thereof), and a prey mite population comprising at least one species from the Phytoseiidae family.

According to yet further embodiments of the different aspects of the present invention, the prey mite species is of the genus Amblyseius, e.g. Amblyseius swirskii.

According to further embodiments of the different aspects of the present invention, the rearing composition comprises immobilized prey mites.

According to further aspects of the present invention, the prey mites are immobilized or dead mites.

According to further aspects, the present invention provides a method for controlling a crop pest, the method comprising applying a composition as defined in any of the above to a field crop.

According to further aspects, the present invention provides use of the composition as defined in any of the above for controlling a crop pest.

According to further aspects, the present invention provides a biological control product for controlling crop pests comprising a mixture of (a) Phytoseiulus persimilis predatory mite individuals species from the Phytoseiidae family (preferably comprising any one of the herein disclosed genetic markers or any combination thereof) raised by the composition as defined in any of the above, (b) prey mite individuals comprising at least one species from the order Astigmata, and (c) optionally a carrier material.

The present invention further provides a slow release system (e.g. sachet) for mites, especially for mite species of the genus Phytoseiulus, particularly Phytoseiulus persimilis (P. persimilis) (preferably comprising any one of the herein disclosed genetic markers or any combination thereof) configured to be applied on a crop.

A core aspect of the innovative solution is that the predatory mites (preferably comprising any one of the herein disclosed genetic markers or any combination thereof) can reproduce within the system for several generations, while a certain proportion of the predatory mites continuously leaves the system and reaches the crop to control pests. This provides a continuous supply of mites to the crop without the need to apply them repeatedly by the farmer.

Embodiments of the slow release system provided by the present invention are based upon the following features:
1. Predatory mite individuals—P. persimilis or other mite species of the Phytoseiulus genus (preferably comprising any one of the herein disclosed genetic markers or any combination thereof).
2. Food source for the predatory mites—a factitious prey or host for example, frozen eggs of Carpoglyphus lactis (C. lactis) or another astigmatic mite.

The predatory mites are combined with their factitious host at the same physical location.

This is done by the following alternative approaches:
a. Providing the predatory mites (preferably comprising any one of the herein disclosed genetic markers or any combination thereof) with their factitious host in a container such as a sachet, packet, pouch, pocket, sack or a bag configured to be hung on the crop plant, from which the mites would slowly and continuously be released to the crop during a period of about three weeks.
b. Applying a mixture containing the predatory mites (preferably comprising any one of the herein disclosed genetic markers or any combination thereof), a carrier and the factitious host as a food source, directly on the crop leaves. From this mixture, the predatory mites would disperse in the crop.

It is noted that such slow release systems for predatory mites are highly desirable for P. persimilis since, P. persimilis was known as a specialist natural enemy of spider mites and therefore reared upon spider mites diet. However, spider mites are not suitable to be used in this kind of mite release systems for crop protection for the following reasons:
Spider mites are pests themselves, and if applied alive, they may damage the crop.
Spider mites cannot reproduce without being supplied with plant material, therefore can't reproduce in a sachet.
Without being supplied with a food source, living spider mites die rapidly and shrivel (e.g. within few days).
If served dead, spider mites quickly shrivel and loose their nutritional value.
Spider mites are expensive to produce.

The present invention enables the use of non-hatching (immobilized), in particular frozen, eggs of C. lactis or other Astigmatid mite species as a factitious host for P. persimilis. Contrary to spider mites, non-hatching eggs (e.g. due to immobilization by freezing) of Astigmatids, in particular C. lactis, maintain their nutritional value for about three weeks. This innovative solution enables the release of P. persimilis predatory mites (preferably comprising any one of the herein disclosed genetic markers or any combination thereof) from a container or a mixture combining the predatory mite (preferably comprising any one of the herein disclosed genetic markers or any combination thereof) with its factitious host, applied on the crop plant for controlling pests.

The present invention provides for the first time genomic sequences and markers that differentiate desirable modified or cultivated P. persimilis populations and lines that are adapted and selected for development and reproduction on non-Tetranychid arthropod prey as the sole food source, from the naturally occurring and non-modified modified or non-cultivated P. persimilis individuals using and reproducing on Tetranychid arthropod prey, that lack the genomic sequences and markers provided herewith.

It is evident by the results disclosed herein that the present invention provides novel and unique molecular markers for identifying a P. persimilis population characterized by improved and desirable development and reproduction capacity on an alternative diet of non-Tetranychid mite species. Each one of the identified genetic variants (SNPs) or any of their combinations may be in a homozygous configuration or in a heterozygous configuration.

According to further aspects, the present invention provides robust technique for examining whole genome sequence of populations of mites and molecular markers for multiple loci by using individual mites. The P. persimilis DNA template generated by the inventors can be used for multiple PCR reactions useful for genetic studies requiring the genotypes of individual mites.

The genetic profiling results of the present invention summarized in Table 1 (and primer sequences for identifying the genetic markers in Table 2) present newly identified haplotypes comprising combinations of genetic variants depicted in these tables. The haplotype combinations are unique for and associated with all tested P+ type P. persimilis populations. The newly identified genetic variants can be used to identify, detect, select and/or screen for the P+ type populations and lines which exhibits significantly improved development and reproduction parameters preying on Astigmatid species, as compared with the non-P+ populations type (P−), which herein show a different genetic profile and allelic haplotype. These development and reproduction parameters may include increased daily reproduction rate, increased daily oviposition rate, high percentage of female individuals capable of reproduction on the non-Tetranychid arthropod prey, and enhanced juvenile and adult female survival rate. The selected P. persimilis line of the present invention exhibits a similar and even improved ability to control their natural prey, spider mites.

The present invention provides genomic markers and sequences uniquely associated with and capable of detecting a P. persimilis populations adapted to and exhibiting increased yield and high reproduction characteristics when reared on non-Tetranychid arthropod prey, such as Astigmatid mite species, as compared to P. persimilis populations absent of the unique genomic markers and sequences. Therefore, these biomarkers are useful in detecting P. persimilis, which exhibits highly efficient development and reproduction on non-phytophagous prey, and screening out P. persimilis populations with low ability to develop and reproduce on such a non-Tetranychid alternative prey.

According to one embodiment, the present invention provides a biological control composition comprising a cultivated rearing population of Phytoseiulus persimilis (P. persimilis) predatory mite individuals, and optionally a carrier material, wherein at least 5% of said P. persimilis individuals comprise within their genome at least one Single Nucleotide Polymorphism (SNP) marker selected from: (a) SNP at position 34739 of SEQ ID NO:84 or a variant, portion or complementary sequence thereof, (b) at least one SNP at a position selected from 105452, 109775, 115020, 115073, 125940, 131548 and 131555, of SEQ ID NO:85 or a variant, portion or complementary sequence thereof, (c) at least one SNP at a position selected from 99333, 99595, 99605, 115325 and 115354, of SEQ ID NO:86 or a variant, portion or complementary sequence thereof, (d) at least one SNP at a position selected from 4508, 11697, 12229, 13135, 32979, 38081, 38220, of SEQ ID NO:87 or a variant, portion or complementary sequence thereof, (e) at least one SNP at a position selected from 30913, 34950, 38409, 38458, 42427, 42604 and 43158 of SEQ ID NO:88 or a variant, portion or complementary sequence thereof, (f) SNP at position 1032 of SEQ ID NO:89 or a variant, portion or complementary sequence thereof, (g) SNP at position 173977 and/or SNP at position 228195 of SEQ ID NO:90 or a variant, portion or complementary sequence thereof, and (h) at least one SNP at a position selected from 857, 15689, 40135 and 49492 of SEQ ID NO:91 or a variant, portion or complementary sequence thereof.

According to a further embodiment, the present invention provides the biological control composition as defined above, wherein the at least one SNP is selected from: (a) SNP of G at position 34739 of SEQ ID NO:84 or a variant, portion or complementary sequence thereof, (b) at least one SNP selected from C at position 105452, G at position 109775, A at position 115020, T at position 115073, C at position 125940, G at position 131548, G at position 131555, of SEQ ID NO:85 or a variant, portion or complementary sequence thereof, (c) at least one SNP selected from A at position 99333, G at position 99595, T at position 99605, A at position 115325, C at position 115354, of SEQ ID NO:86 or a variant, portion or complementary sequence thereof, (d) at least one SNP selected from A at position 4508, A at position 11697, C at position 12229, A at position 13135, C at position 32979, G at position 38081, C at position 38220 of SEQ ID NO:87 or a variant, portion or complementary sequence thereof, (e) at least one SNP selected from A at position 30913, C at position 34950, C at position 38409, A at position 38458, T at position 42427, G at position 42604, T at position 43158 of SEQ ID NO:88 or a variant, portion or complementary sequence thereof, (f) SNP of G at position 1032 of SEQ ID NO:89 or a variant, portion or complementary sequence thereof, (g) SNP of A at position 173977 and/or SNP of T at position 228195 of SEQ ID NO:90 or a variant, portion or complementary sequence thereof, and (h) at least one SNP selected from C at position 857, T at position 15689, C at position 40135, G at position 49492 of SEQ ID NO:91 or a variant, portion or complementary sequence thereof.

According to a further embodiment, the present invention provides the biological control composition as defined in any above, wherein said variant comprises at least 90% sequence similarity to the corresponding SEQ ID nucleotide sequence.

According to a further embodiment, the present invention provides the biological control composition as defined in any of the above, wherein at least 5.5%, such as at least 10%, 20%, 30%, 40%, 50%, 60%, 70%, 80%, at least 90% or 100% of said P. persimilis predatory individuals of said cultivated population comprise said at least one SNP marker.

According to a further embodiment, the present invention provides the biological control composition as defined in any of the above, wherein the at least 5% of said P. persimilis predatory individuals comprise within their genome a polynucleotide genomic sequence selected from SEQ ID NO:1-28 and SEQ ID NO:84-91, and any combination thereof.

According to a further embodiment, the present invention provides the biological control composition as defined in any of the above, wherein the at least 5% of said P. persimilis predatory individuals comprise within their genome a polynucleotide genomic sequence selected from SEQ ID NO:1 to SEQ ID NO:91, and any combination thereof.

According to a further embodiment, the present invention provides the biological control composition as defined in any of the above, wherein the cultivated rearing population of P. persimilis predatory mite individuals is characterized by improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

According to a further embodiment, the present invention provides the biological control composition as defined in any of the above, wherein the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

According to a further embodiment, the present invention provides the biological control composition as defined in any of the above, wherein the composition further comprises as a food source for the P. persimilis predatory mites of the cultivated rearing population, a non-Tetranychid arthropod prey, preferably an immobilized non-Tetranychid arthropod prey, such as on an Astigmatid prey, most preferably on an immobilized Astigmatid prey.

According to a further embodiment, the present invention provides the biological control composition as defined in any of the above, wherein the non-Tertranychid arthropod prey is selected from an Astigmatid mite species, an Artemia species, species from the Phytoseiidae family, Thrips such as Frankliniella occidentalis, Tarsonemidae mite species, and lepidopteran eggs such as of Ephestia kunhiela.

According to a further embodiment, the present invention provides the biological control composition as defined in any of the above, wherein said Astigmata prey species is selected from members of the family Acaridae, Carpoglyphidae, Glycyphagidae, and/or from the family Chortoglyphagidae.

According to a further embodiment, the present invention provides the biological control composition as defined in any of the above, wherein the at least one species from the order Astigmata is selected from members of a genus such as Acarus, Tyrophagus, Aleuroglyphus, Lardoglyphus, Caloglyphus, Suidasia, Thyreophagus, Carpoglyphus, Glycyphagus, Lepidoglyphus, Blomia, and/or Chortoglyphus.

According to a further embodiment, the present invention provides the biological control composition as defined in any of the above, wherein the at least one species from the order Astigmata is selected from: Acarus siro, Acarusfarris, Acarus immobilis, Acarus chaetoxysilos, Tyrophagus longior, Tyrophagus similis, Tyrophagus putrescentiae, Tyrophagus communis, Aleuroglyphus ovatus, Lardoglyphus konoi, Caloglyphus mycophagus, Suidasia nesbitti, Thyreophagus entomophagus, Carpoglyphus lactis, Carpoglyphus munroi, Glycyphagus domesticus, Lepidoglyphus destructor, Blomia freeman, Blomia tropicalis and/or Chortoglyphus arcuatus.

According to a further embodiment, the present invention provides the biological control composition as defined in any of the above, wherein said Astigmatid mite species is selected from:
i) Carpoglyphidae such as from the genus Carpoglyphus e.g. Carpoglyphus lactis;
ii) Pyroglyphidae such as from the genus Dermatophagoides e.g. Dermatophagoides pteronysinus, Dermatophagoides farinae; from the genus Euroglyphus e.g. Euroglyphus longior, Euroglyphus maynei; from the genus Pyroglyphus e.g. Pyroglyphus africanus;
iii) Glycyphagidae such as from the subfamily Ctenoglyphinae, such as from the genus Diamesoglyphus e.g. Diamesoglyphus intermedius, or from the genus Ctenoglyphus, e.g. Ctenoglyphus plumiger, Ctenoglyphus canestrinii, Ctenoglyphus palmifer; the subfamily Glycyphaginae, such as from the genus Blomia, e.g. Blomia freemani or from the genus Glycyphagus, e.g. Glycyphagus ornatus, Glycyphagus bicaudatus, Glycyphagus privatus, Glycyphagus domesticus, or from the genus Lepidoglyphus e.g. Lepidoglyphus michaeli, Lepidoglyphus fustifer, Lepidoglyphus destructor, or from the genus Austroglycyphagus, e.g. Austroglycyphagus geniculatus; from the subfamily Airoglyphinae, such as from the genus Airoglyphus, e.g. Airoglyphus robustus; from the subfamily Labidophorinae, such as from the genus Gohieria, e.g. Gohieria. fusca; or from the subfamily Nycteriglyphinae such as from the genus Coproglyphus, e.g. Coproglyphus stammeri or from the subfamily Chortoglyphidae, such as the genus Chortoglyphus e.g. Chortoglyphus arcuatus;
iv) Acaridae such as from the genus Tyrophagus e.g. Tyrophagus putrescentiae, Tyrophagus tropicus, Tyrophagus communis from the genus Acarus e.g. Acarus siro, Acarus farris, Acarus gracilis; from the genus Lardoglyphus e.g. Lardoglyphus konoi, from the genus Thyreophagus, such as Thyreophagus entomophagus; from the genus Aleuroglyphus, e.g. Aleuroglyphus ovatus;
v) Suidasiidae such as from the genus Suidasia, such as Suidasia nesbiti, Suidasia pontifica or Suidasia medanensis.

According to a further embodiment, the present invention provides the biological control composition as defined in any of the above, wherein the non-Tetranychid arthropod prey comprises immobilized life stages comprising immobilized eggs.

According to a further embodiment, the present invention provides the biological control composition as defined in any of the above, wherein said immobilization is carried out by a treatment selected from: thermal treatment, such as freezing, freeze-drying, heating, cold-shock or heat-shock treatment; chemical treatment, such as gas or fume treatment; radiation treatment, such as UV, microwave, gamma irradiation or X-ray treatment; mechanical treatment, such as vigorous shaking, or stirring, subjecting to shear forces, collision; gas pressure treatment, such as ultrasound treatment, pressure changes, pressure drops; electrical treatment, such as electrocution; immobilizing with an adhesive; immobilization by starvation, such as induced by water or food deprivation; immobilization by suffocation or anoxia treatment, such as by temporarily eliminating oxygen from the atmosphere or replacing oxygen by another gas and any combination thereof.

According to a further embodiment, the present invention provides the biological control composition as defined in any of the above, wherein the improved rearing properties of the cultivated rearing P. persimilis population on a non-Tetranychid arthropod prey, are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals characterized by less than 5% individuals comprising within their genome said at least one SNP marker.

According to a further embodiment, the present invention provides the biological control composition as defined in any of the above, wherein said carrier material is selected from particles or elements of sawdust, vermiculite, bran, wheat bran, buckwheat husks, rice husks or millet husks, or comprising a mixture thereof, or any other inert carrier material and/or any carrier having carrier elements comprising surfaces to which mites can adhere or carrier elements comprising mite shelters.

According to a further embodiment, the present invention provides the biological control composition as defined in any of the above, wherein said composition is contained in a container such as a sachet, a bottle or any other packaging type container or device configured to holding the biological control composition.

According to a further embodiment, the present invention provides a method for obtaining a biological control composition comprising a cultivated rearing population of Phytoseiulus persimilis (P. persimilis) predatory mite individuals as defined in any of the above, wherein said method comprises steps of: (a) providing a rearing population of a P. persimilis predatory mite species, the rearing population comprising individuals of the P. persimilis species, reared on a suitable food source for the Phytoseiulus individuals, said food source comprising a prey species selected from the Tetranychidae species; (b) providing a population of individuals of preselected non-Tetranychid arthropod species, preferably an Astigmatid mite species, most preferably an immobilized Astigmatid mite species having immobilized life stages comprising immobilized eggs; (c) rearing the P. persimilis individuals on the preselected non-Tetranychid arthropod species as a food source; (d) screening the genome of a sample of P. persimilis individuals for the presence of at least 5% of at least one SNP selected from: (i) SNP of G at position 34739 of SEQ ID NO:84 or a variant, portion or complementary sequence thereof, (ii) at least one SNP selected from C at position 105452, G at position 109775, A at position 115020, T at position 115073, C at position 125940, G at position 131548, G at position 131555, of SEQ ID NO:85 or a variant, portion or complementary sequence thereof, (iii) at least one SNP selected from A at position 99333, G at position 99595, T at position 99605, A at position 115325, C at position 115354, of SEQ ID NO:86 or a variant, portion or complementary sequence thereof, (iv) at least one SNP selected from A at position 4508, A at position 11697, C at position 12229, A at position 13135, C at position 32979, G at position 38081, C at position 38220 of SEQ ID NO:87 or a variant, portion or complementary sequence thereof, (v) at least one SNP selected from A at position 30913, C at position 34950, C at position 38409, A at position 38458, T at position 42427, G at position 42604, T at position 43158 of SEQ ID NO:88 or a variant, portion or complementary sequence thereof, (vi) SNP of G at position 1032 of SEQ ID NO:89 or a variant, portion or complementary sequence thereof, (vii) SNP of A at position 173977 and/or SNP of T at position 228195 of SEQ ID NO:90 or a variant, portion or complementary sequence thereof, and (viii) at least one SNP selected from C at position 857, T at position 15689, C at position 40135, G at position 49492 of SEQ ID NO:91 or a variant, portion or complementary sequence thereof; (e) optionally, alternatingly rearing selected Phytoseiulus individuals in a sequence of:
rearing for at least 2, such as between 5 and 50, generations while using a food source comprising the preselected non-Tetranychid arthropod species;
rearing for at least 2, such as between 5 and 50, generations while using a food source comprising the prey species selected from the Tetranychidae
screening the genome of a sample of P. persimilis individuals for the presence of at least 5% of the at least one SNP.

According to a further embodiment, the present invention provides the method as defined above, further comprises steps of: (a) separating eggs and/or juvenile developmental stages such as larvae and/or nymphs from the preselected non-Tetranychid arthropod prey rearing population; (b) mixing the separated eggs and/or juvenile developmental stages such as larvae and/or nymphs with a carrier material, such as a carrier material selected from sawdust, wheat bran, buckwheat husks, rice husks or millet husks, or comprising a mixture thereof; (c) freezing the mixture of step (b); and (d) rearing the predatory mite individuals on the mixture as a food source.

According to a further embodiment, the present invention provides the method as defined in any of the above, wherein the provided rearing population is a population composed of a number of sub-populations, wherein said sub-populations are from distinct sources, such as from distinct production populations and/or from natural populations isolated from distinct geographical locations.

According to a further embodiment, the present invention provides the method as defined in any of the above, wherein the provided rearing population comprises at least 100 individuals, such as between 200 and 5000 individuals, preferably between 500 and 1500 individuals.

According to a further embodiment, the present invention provides the method as defined in any of the above, wherein the sample for genome screening comprises at least 50 individuals, such as between 100 and 300 individuals.

According to a further embodiment, the present invention provides use of a biological control composition as defined in any of the above, for controlling a pest in a crop.

According to a further embodiment, the present invention provides a method for controlling a pest in a crop comprising providing the crop with a biological control composition as defined in any of the above.

According to a further embodiment, the present invention provides a method for screening for or identifying a biological control composition as defined in any of the above, the method comprises steps of: (a) obtaining a sample of said population of individuals, the sample preferably comprise at least 50 individuals; (b) pooling the genome said individuals and screening the pooled genome for the presence of at least 5% of at least one SNP selected from: (i) SNP of G at position 34739 of SEQ ID NO:84 or a variant, portion or complementary sequence thereof, (ii) at least one SNP selected from C at position 105452, G at position 109775, A at position 115020, T at position 115073, C at position 125940, G at position 131548, G at position 131555, of SEQ ID NO:85 or a variant, portion or complementary sequence thereof, (iii) at least one SNP selected from A at position 99333, G at position 99595, T at position 99605, A at position 115325, C at position 115354, of SEQ ID NO:86 or a variant, portion or complementary sequence thereof, (iv) at least one SNP selected from A at position 4508, A at position 11697, C at position 12229, A at position 13135, C at position 32979, G at position 38081, C at position 38220 of SEQ ID NO:87 or a variant, portion or complementary sequence thereof, (v) at least one SNP selected from A at position 30913, C at position 34950, C at position 38409, A at position 38458, T at position 42427, G at position 42604, T at position 43158 of SEQ ID NO:88 or a variant, portion or complementary sequence thereof, (vi) SNP of G at position 1032 of SEQ ID NO:89 or a variant, portion or complementary sequence thereof, (vii) SNP of A at position 173977 and/or SNP of T at position 228195 of SEQ ID NO:90 or a variant, portion or complementary sequence thereof, and (viii) at least one SNP selected from C at position 857, T at position 15689, C at position 40135, G at position 49492 of SEQ ID NO:91 or a variant, portion or complementary sequence thereof.

According to a further embodiment, the present invention provides a cultivated Phytoseiulus persimilis (P. persimilis) mite individual, wherein said cultivated Phytoseiulus predatory individual comprises within its genome at least one single nucleotide polymorphism (SNP) marker selected from:
a. SNP1 at position corresponding to position 163 of SEQ ID NO:1 or a variant, portion or complementary sequence thereof,
b. SNP2 at position 181 of SEQ ID NO:2 or a variant, portion or complementary sequence thereof,
c. SNP3 at position 197 of SEQ ID NO:3 or a variant, portion or complementary sequence thereof,
d. SNP4 at position 111 and/or SNP5 at position 165 of SEQ ID NO:4 or a variant, portion or complementary sequence thereof,
e. SNP6 at position 193 of SEQ ID NO:5 or a variant, portion or complementary sequence thereof,
f. SNP7 at position 186 and/or SNP8 at position 193 of SEQ ID NO:6 or a variant, portion or complementary sequence thereof,
g. SNP9 at position 161 of SEQ ID NO:7 or a variant, portion or complementary sequence thereof,
h. SNP10 at position 203 and/or SNP11 at position 213 of SEQ ID NO:8 or a variant, portion or complementary sequence thereof,
i. SNP12 at position 163 and/or SNP13 at position 192 of SEQ ID NO:9 or a variant, portion or complementary sequence thereof,
j. SNP14 at position 208 of SEQ ID NO:10 or a variant, portion or complementary sequence thereof,
k. SNP15 at position 199 of SEQ ID NO:11 or a variant, portion or complementary sequence thereof,
l. SNP16 at position 266 of SEQ ID NO:12 or a variant, portion or complementary sequence thereof,
m. SNP17 at position 216 of SEQ ID NO:13 or a variant, portion or complementary sequence thereof,
n. SNP18 at position 187 of SEQ ID NO:14 or a variant, portion or complementary sequence thereof,
o. SNP19 at position 279 of SEQ ID NO:15 or a variant, portion or complementary sequence thereof,
p. SNP20 at position 188 of SEQ ID NO:16 or a variant, portion or complementary sequence thereof,
q. SNP21 at position 165 of SEQ ID NO:17 or a variant, portion or complementary sequence thereof,
r. SNP22 at position 183 of SEQ ID NO:18 or a variant, portion or complementary sequence thereof,
s. SNP23 at position 133 and/or SNP24 at position 182 of SEQ ID NO:19 or a variant, portion or complementary sequence thereof,
t. SNP25 at position 146 and/or SNP26 position 323 of SEQ ID NO:20 or a variant, portion or complementary sequence thereof,
u. SNP27 at position 241 of SEQ ID NO:21 or a variant, portion or complementary sequence thereof,
v. SNP28 at position 236 of SEQ ID NO:22 or a variant, portion or complementary sequence thereof,
w. SNP29 at position 199 of SEQ ID NO:23 or a variant, portion or complementary sequence thereof,
x. SNP30 at position 185 of SEQ ID NO:24 or a variant, portion or complementary sequence thereof,
y. SNP31 at position 158 of SEQ ID NO:25 or a variant, portion or complementary sequence thereof,
z. SNP32 at position 143 of SEQ ID NO:26 or a variant, portion or complementary sequence thereof,
aa. SNP33 at position 157 of SEQ ID NO:27 or a variant, portion or complementary sequence thereof, and
bb. SNP34 at position 161 of SEQ ID NO:28 or a variant, portion or complementary sequence thereof, and any combination thereof.

According to a further embodiment, the present invention provides the cultivated P. persimilis mite individual as defined in any of the above, wherein said variant comprises at least 90% sequence similarity to the corresponding nucleotide SEQ ID sequence.

According to a further embodiment, the present invention provides the cultivated P. persimilis mite individual as defined in any of the above, wherein said P. persimilis predatory individual comprising within it's genome a polynucleotide sequence selected from SEQ ID NO:1 to SEQ ID NO:28, and any combination thereof.

According to a further embodiment, the present invention provides the cultivated P. persimilis mite individual as defined in any of the above, wherein the at least one SNP is detected using primers comprising pairs of sequences selected from: SEQ ID NO:29 and SEQ ID NO:30 for detection of SNP1, SEQ ID NO:31 and SEQ ID NO:32 for detection of SNP2, SEQ ID NO:33 and SEQ ID NO:34 for detection of SNP3, SEQ ID NO:35 and SEQ ID NO:36 for detection of SNP4-5, SEQ ID NO:37 and SEQ ID NO:38 for detection of SNP6, SEQ ID NO:39 and SEQ ID NO:40 for detection of SNP7-8, SEQ ID NO:41 and SEQ ID NO:42 for detection of SNP9, SEQ ID NO:43 and SEQ ID NO:44 for detection of SNP10-11, SEQ ID NO:45 and SEQ ID NO:46 for detection of SNP12-13, SEQ ID NO:47 and SEQ ID NO:48 for detection of SNP14, SEQ ID NO:49 and SEQ ID NO:50 for detection of SNP15-16, SEQ ID NO:51 and SEQ ID NO:50 for detection of SNP16, SEQ ID NO:52 and SEQ ID NO:53 for detection of SNP17, SEQ ID NO:54 and SEQ ID NO:55 for detection of SNP18, SEQ ID NO:56 and SEQ ID NO:57 for detection of SNP19, SEQ ID NO:58 and SEQ ID NO:59 for detection of SNP20, SEQ ID NO:60 and SEQ ID NO:61 for detection of SNP21, SEQ ID NO:62 and SEQ ID NO:63 for detection of SNP22, SEQ ID NO:64 and SEQ ID NO:65 for detection of SNP23-24, SEQ ID NO:66 and SEQ ID NO:67 for detection of SNP25-26, SEQ ID NO:68 and SEQ ID NO:69 for detection of SNP27, SEQ ID NO:70 and SEQ ID NO:71 for detection of SNP28, SEQ ID NO:72 and SEQ ID NO:73 for detection of SNP29, SEQ ID NO:74 and SEQ ID NO:75 for detection of SNP30, SEQ ID NO:76 and SEQ ID NO:77 for detection of SNP31, SEQ ID NO:78 and SEQ ID NO:79 for detection of SNP32, SEQ ID NO:80 and SEQ ID NO:81 for detection of SNP33, and SEQ ID NO:82 and SEQ ID NO:83 for detection of SNP34.

According to a further embodiment, the present invention provides an isolated cell of the cultivated Phytoseiulus persimilis (P. persimilis) predatory mite as defined in any of the above.

According to a further embodiment, the present invention provides a Phytoseiulus persimilis rearing mite population, wherein at least 5% of the population are the cultivated P. persimilis mite individuals as defined in any of the above.

According to a further embodiment, the present invention provides the Phytoseiulus persimilis rearing mite population as defined in any of the above, wherein the population further comprises a food source for the P. persimilis predatory mites, said food source comprises a non-Tetranychid arthropod as a factitious host, preferably an immobilized non-Tetranychid arthropod prey, such as on an Astigmatid prey, most preferably on an immobilized Astigmatid prey.

According to a further embodiment, the present invention provides the Phytoseiulus persimilis rearing mite population as defined in any of the above, wherein the population is characterized by improved rearing properties on a non-Tetranychid arthropod prey as a food source, preferably on an immobilized non-Tetranychid arthropod prey, such as on an Astigmatid prey, most preferably on an immobilized Astigmatid prey.

According to a further embodiment, the present invention provides the Phytoseiulus persimilis rearing mite population as defined in any of the above, wherein the improved rearing properties on a non-Tetranychid arthropod prey are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals characterized by less than 5% individuals comprising within their genome said at least one SNP marker.

According to a further embodiment, the present invention provides the Phytoseiulus persimilis rearing mite population as defined in any of the above, wherein the population is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

According to a further embodiment, the present invention provides use of a Phytoseiulus persimilis rearing mite population as defined in any of the above, for controlling a pest in a crop.

According to a further embodiment, the present invention provides a method for controlling a pest in a crop comprising providing the crop with a Phytoseiulus persimilis rearing mite population as defined in any of the above.

According to a further embodiment, the present invention provides device for releasing mite individuals of a Phytoseiulus persimilis predatory mite species, said device comprising a container holding a biological control composition as defined in any of the above, or a Phytoseiulus persimilis rearing mite population as defined in any of the above, wherein the device comprises an exit for mobile life stages of the P. persimilis predatory mite species, preferably an exit suitable for providing a sustained release of a number of mobile life stages.

According to a further embodiment, the present invention provides use of a biological control composition as defined in any of the above, or a Phytoseiulus persimilis rearing mite population as defined in any of the above, preferably in a device as defined in any of the above, for crop protection.

According to a further embodiment, the present invention provides use of a non-Tetranychid arthropod species, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, as a food source, preferably as a rearing prey, for a Phytoseiulus persimilis rearing mite population as defined in any of the above.

According to a further embodiment, the present invention provides the use as defined in any of the above, wherein said use comprises applying individuals of a non-Tetranychid arthropod species to a target plant, preferably an immobilized non-Tetranychid arthropod species, such as a non-phytophagous prey, preferably an Astigmatid species, most preferably an immobilized Astigmatid species, such as most preferably an immobilized Astigmatid species, in particular a Carpoglyhus species, having immobilized life stages comprising immobilized eggs, or a mixture of immobilized life stages comprising eggs and mobile stages of the non-Tetranychid arthropod species.

According to a further embodiment, the present invention provides a method for identifying and/or selecting Phytoseiulus persimilis (P. persimilis) predatory mite individuals characterized by improved rearing properties on a non-Tetranychid arthropod prey as a food source, preferably on an immobilized non-Tetranychid arthropod prey, such as on an Astigmatid prey, most preferably on an immobilized Astigmatid prey, wherein said method comprises detecting in the genome of the P. persimilis predatory mite individuals, the presence of a genomic haplotype comprising a single nucleotide polymorphism (SNP) marker at a position corresponding to at least one of: SNP1 at position 163 of SEQ ID NO:1 or a variant, portion or complementary sequence thereof, SNP2 at position 181 of SEQ ID NO:2 or a variant, portion or complementary sequence thereof, SNP3 at position 197 of SEQ ID NO:3 or a variant, portion or complementary sequence thereof, SNP4 at position 111 of SEQ ID NO:4 or a variant, portion or complementary sequence thereof, SNP5 at position 165 of SEQ ID NO:4 or a variant, portion or complementary sequence thereof, SNP6 at position 193 of SEQ ID NO:5 or a variant, portion or complementary sequence thereof, SNP7 at position 186 of SEQ ID NO:6 or a variant, portion or complementary sequence thereof, SNP8 at position 193 of SEQ ID NO:6 or a variant, portion or complementary sequence thereof, SNP9 at position 161 of SEQ ID NO:7 or a variant, portion or complementary sequence thereof, SNP10 at position 203 of SEQ ID NO:8 or a variant, portion or complementary sequence thereof, SNP11 at position 213 of SEQ ID NO:8 or a variant, portion or complementary sequence thereof, SNP12 at position 163 of SEQ ID NO:9 or a variant, portion or complementary sequence thereof, SNP13 at position 192 of SEQ ID NO:9 or a variant, portion or complementary sequence thereof, SNP14 at position 208 of SEQ ID NO:10 or a variant, portion or complementary sequence thereof, SNP15 at position 199 of SEQ ID NO:11 or a variant, portion or complementary sequence thereof, SNP16 at position 266 of SEQ ID NO:12 or a variant, portion or complementary sequence thereof, SNP17 at position 216 of SEQ ID NO:13 or a variant, portion or complementary sequence thereof, SNP18 at position 187 of SEQ ID NO:14 or a variant, portion or complementary sequence thereof, SNP19 at position 279 of SEQ ID NO:15 or a variant, portion or complementary sequence thereof, SNP20 at position 188 of SEQ ID NO:16 or a variant, portion or complementary sequence thereof, SNP21 at position 165 of SEQ ID NO:17 or a variant, portion or complementary sequence thereof, SNP22 at position 183 of SEQ ID NO:18 or a variant, portion or complementary sequence thereof, SNP23 at position 133 of SEQ ID NO:19 or a variant, portion or complementary sequence thereof, SNP24 at position 182 of SEQ ID NO:19 or a variant, portion or complementary sequence thereof, SNP25 at position 146 of SEQ ID NO:20 or a variant, portion or complementary sequence thereof, SNP26 at position 323 of SEQ ID NO:20 or a variant, portion or complementary sequence thereof, SNP27 at position 241 of SEQ ID NO:21 or a variant, portion or complementary sequence thereof, SNP28 at position 236 of SEQ ID NO:22 or a variant, portion or complementary sequence thereof, SNP29 at position 199 of SEQ ID NO:23 or a variant, portion or complementary sequence thereof, SNP30 at position 185 of SEQ ID NO:24 or a variant, portion or complementary sequence thereof, SNP31 at position 158 of SEQ ID NO:25 or a variant, portion or complementary sequence thereof, SNP32 at position 143 of SEQ ID NO:26 or a variant, portion or complementary sequence thereof, SNP33 at position 157 of SEQ ID NO:27 or a variant, portion or complementary sequence thereof, SNP34 at position 161 of SEQ ID NO:28 or a variant, portion or complementary sequence thereof, and any combination thereof.

According to a further embodiment, the present invention provides the method as defined in any of the above, wherein said variant comprises at least 90% similarity to the corresponding nucleotide SEQ ID sequence.

According to a further embodiment, the present invention provides the method as defined in any of the above, wherein said Phytoseiulus predatory individual comprising a polynucleotide genomic sequence selected from SEQ ID NO:1 to SEQ ID NO:28, and any combination thereof.

According to a further embodiment, the present invention provides the method as defined in any of the above, wherein the detection is performed using a primer pair selected from: SEQ ID NO:29 and SEQ ID NO:30 for detection of SNP1, SEQ ID NO:31 and SEQ ID NO:32 for detection of SNP2, SEQ ID NO:33 and SEQ ID NO:34 for detection of SNP3, SEQ ID NO:35 and SEQ ID NO:36 for detection of SNP4-5, SEQ ID NO:37 and SEQ ID NO:38 for detection of SNP6, SEQ ID NO:39 and SEQ ID NO:40 for detection of SNP7-8, SEQ ID NO:41 and SEQ ID NO:42 for detection of SNP9, SEQ ID NO:43 and SEQ ID NO:44 for detection of SNP10-11, SEQ ID NO:45 and SEQ ID NO:46 for detection of SNP12-13, SEQ ID NO:47 and SEQ ID NO:48 for detection of SNP14, SEQ ID NO:49 and SEQ ID NO:50 for detection of SNP15-16, SEQ ID NO:51 and SEQ ID NO:50 for detection of SNP16, SEQ ID NO:52 and SEQ ID NO:53 for detection of SNP17, SEQ ID NO:54 and SEQ ID NO:55 for detection of SNP18, SEQ ID NO:56 and SEQ ID NO:57 for detection of SNP19, SEQ ID NO:58 and SEQ ID NO:59 for detection of SNP20, SEQ ID NO:60 and SEQ ID NO:61 for detection of SNP21, SEQ ID NO:62 and SEQ ID NO:63 for detection of SNP22, SEQ ID NO:64 and SEQ ID NO:65 for detection of SNP23-24, SEQ ID NO:66 and SEQ ID NO:67 for detection of SNP25-26, SEQ ID NO:68 and SEQ ID NO:69 for detection of SNP27, SEQ ID NO:70 and SEQ ID NO:71 for detection of SNP28, SEQ ID NO:72 and SEQ ID NO:73 for detection of SNP29, SEQ ID NO:74 and SEQ ID NO:75 for detection of SNP30, SEQ ID NO:76 and SEQ ID NO:77 for detection of SNP31, SEQ ID NO:78 and SEQ ID NO:79 for detection of SNP32, SEQ ID NO:80 and SEQ ID NO:81 for detection of SNP33, SEQ ID NO:82 and SEQ ID NO:83 for detection of SNP34 and any combination of primer pairs.

According to a further embodiment, the present invention provides the method as defined in any of the above, wherein the improved rearing properties on a non-Tetranychid arthropod prey are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals lacking the at least one SNP and or the polynucleotide sequence selected from SEQ ID NO:1 to SEQ ID NO:28.

According to a further embodiment, the present invention provides a method for identifying and/or selecting a cultivated rearing population of Phytoseiulus persimilis (P. persimilis) predatory mite individuals characterized by improved rearing properties on a non-Tetranychid arthropod prey as a food source, preferably on an immobilized non-Tetranychid arthropod prey, such as on an Astigmatid prey, most preferably on an immobilized Astigmatid prey, wherein said method comprises detecting in the genome of at least 5% of said P. persimilis individuals at least one Single Nucleotide Polymorphism (SNP) marker selected from:
a. SNP of G at position 34739 of SEQ ID NO:84 or a variant, portion or complementary sequence thereof,
b. at least one SNP selected from C at position 105452, G at position 109775, A at position 115020, T at position 115073, C at position 125940, G at position 131548, G at position 131555, of SEQ ID NO:85 or a variant, portion or complementary sequence thereof,
c. at least one SNP selected from A at position 99333, G at position 99595, T at position 99605, A at position 115325, C at position 115354, of SEQ ID NO:86 or a variant, portion or complementary sequence thereof,
d. at least one SNP selected from A at position 4508, A at position 11697, C at position 12229, A at position 13135, C at position 32979, G at position 38081, C at position 38220 of SEQ ID NO:87 or a variant, portion or complementary sequence thereof,
e. at least one SNP selected from A at position 30913, C at position 34950, C at position 38409, A at position 38458, T at position 42427, G at position 42604, T at position 43158 of SEQ ID NO:88 or a variant, portion or complementary sequence thereof,
f. SNP of G at position 1032 of SEQ ID NO:89 or a variant, portion or complementary sequence thereof,
g. SNP of A at position 173977 and/or SNP of T at position 228195 of SEQ ID NO:90 or a variant, portion or complementary sequence thereof, and
h. at least one SNP selected from C at position 857, T at position 15689, C at position 40135, G at position 49492 of SEQ ID NO:91 or a variant, portion or complementary sequence thereof.

According to a further embodiment, the present invention provides the method as defined in any of the above, wherein said variant comprises at least 90% sequence similarity to the corresponding SEQ ID nucleotide sequence.

According to a further embodiment, the present invention provides the method as defined in any of the above, wherein at least 5.5%, such as at least 10%, 20%, 30%, 40%, 50%, 60%, 70%, 80%, at least 90% or 100% of said P. persimilis predatory individuals of said cultivated population comprise said at least one SNP marker.

According to a further embodiment, the present invention provides the method as defined in any of the above, wherein the at least 5% of said P. persimilis predatory individuals comprise within their genome a polynucleotide genomic sequence selected from SEQ ID NO:1-28 and SEQ ID NO:84-91, and any combination thereof.

According to a further embodiment, the present invention provides the method as defined in any of the above, wherein the at least 5% of said P. persimilis predatory individuals comprise within their genome a polynucleotide genomic sequence selected from SEQ ID NO:1 to SEQ ID NO:91, and any combination thereof.

According to a further embodiment, the present invention provides the method as defined in any of the above, wherein the cultivated rearing population of P. persimilis predatory mite individuals is characterized by improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

According to a further embodiment, the present invention provides the method as defined in any of the above, wherein the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

According to a further embodiment, the present invention provides a method for obtaining a Phytoseiulus persimilis rearing mite population characterized by improved rearing properties on a non-Tetranychid arthropod prey as a factious host, preferably on an immobilized non-Tetranychid arthropod prey, such as on an Astigmatid prey, most preferably on an immobilized Astigmatid prey, wherein at least 5% of the population are the cultivated P. persimilis mite individuals as defined in any of the above, said method comprises steps of:
a. providing a rearing population of a P. persimilis predatory mite species, the rearing population comprising individuals of the P. persimilis species, reared on a suitable food source for the Phytoseiulus individuals, said food source comprising a prey species selected from the Tetranychidae species;
b. providing a population of individuals of preselected non-Tetranychid arthropod species, preferably an Astigmatid mite species, most preferably an immobilized Astigmatid mite species having immobilized life stages comprising immobilized eggs;
c. rearing the P. persimilis individuals on the preselected non-Tetranychid arthropod species as a food source;
d. screening the genome of a sample of P. persimilis individuals for the presence of at least 5% of at least one SNP selected from:
   i SNP of G at position 34739 of SEQ ID NO:84 or a variant, portion or complementary sequence thereof,
   ii at least one SNP selected from C at position 105452, G at position 109775, A at position 115020, T at position 115073, C at position 125940, G at position 131548, G at position 131555, of SEQ ID NO:85 or a variant, portion or complementary sequence thereof,
   iii at least one SNP selected from A at position 99333, G at position 99595, T at position 99605, A at position 115325, C at position 115354, of SEQ ID NO:86 or a variant, portion or complementary sequence thereof,
   iv at least one SNP selected from A at position 4508, A at position 11697, C at position 12229, A at position 13135, C at position 32979, G at position 38081, C at position 38220 of SEQ ID NO:87 or a variant, portion or complementary sequence thereof,
   v at least one SNP selected from A at position 30913, C at position 34950, C at position 38409, A at position 38458, T at position 42427, G at position 42604, T at position 43158 of SEQ ID NO:88 or a variant, portion or complementary sequence thereof,
   vi SNP of G at position 1032 of SEQ ID NO:89 or a variant, portion or complementary sequence thereof,
   vii SNP of A at position 173977 and/or SNP of T at position 228195 of SEQ ID NO:90 or a variant, portion or complementary sequence thereof, and
   viii at least one SNP selected from C at position 857, T at position 15689, C at position 40135, G at position 49492 of SEQ ID NO:91 or a variant, portion or complementary sequence thereof;
e. optionally, alternatingly rearing selected Phytoseiulus individuals in a sequence of:
   rearing for at least 2, such as between 5 and 50, generations while using a food source comprising the preselected non-Tetranychid arthropod species;
   rearing for at least 2, such as between 5 and 50, generations while using a food source comprising the prey species selected from the Tetranychidae
   screening the genome of a sample of P. persimilis individuals for the presence of at least 5% of the at least one SNP.

According to a further embodiment, the present invention provides the method as defined in any of the above, further comprises steps of:
a. separating eggs and/or juvenile developmental stages such as larvae and/or nymphs from the preselected non-Tetranychid arthropod prey rearing population;
b. mixing the separated eggs and/or juvenile developmental stages such as larvae and/or nymphs with a carrier material, such as a carrier material selected from sawdust, wheat bran, buckwheat husks, rice husks or millet husks, or comprising a mixture thereof;
c. freezing the mixture of step (b); and
d. rearing the predatory mite individuals on the mixture as a food source.

According to a further embodiment, the present invention provides the method as defined in any of the above, wherein the provided rearing population is a population composed of a number of sub-populations, wherein said sub-populations are from distinct sources, such as from distinct production populations and/or from natural populations isolated from distinct geographical locations.

According to a further embodiment, the present invention provides the method as defined in any of the above, wherein the provided rearing population comprises at least 100 individuals, such as between 200 and 5000 individuals, preferably between 500 and 1500 individuals.

According to a further embodiment, the present invention provides the method as defined in any of the above, wherein the sample for genome screening comprises at least 50 individuals, such as between 100 and 300 individuals.

According to a further embodiment, the present invention provides the method as defined in any of the above, wherein the cultivated rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

According to a further embodiment, the present invention provides an isolated nucleic acid sequence comprising at least 90% similarity to a polynucleotide sequence selected from SEQ ID NO:1 to SEQ ID NO:91 or a portion thereof and any combination thereof.

According to a further embodiment, the present invention provides use of a nucleic acid sequence selected from SEQ ID NO:84 to SEQ ID NO:91 or a portion thereof to identify a rearing population of Phytoseiulus persimilis (P. persimilis) predatory mite individuals characterized by improved rearing properties on a non-Tetranychid arthropod prey as a factitious host, preferably on an immobilized non-Tetranychid arthropod prey, such as on an Astigmatid prey, most preferably on an immobilized Astigmatid prey, wherein at least 5% of said P. persimilis individuals comprise within their genome at least one Single Nucleotide Polymorphism (SNP) marker selected from:
a. SNP of G at position 34739 of SEQ ID NO:84 or a variant, portion or complementary sequence thereof,
b. at least one SNP selected from C at position 105452, G at position 109775, A at position 115020, T at position 115073, C at position 125940, G at position 131548, G at position 131555, of SEQ ID NO:85 or a variant, portion or complementary sequence thereof,
c. at least one SNP selected from A at position 99333, G at position 99595, T at position 99605, A at position 115325, C at position 115354, of SEQ ID NO:86 or a variant, portion or complementary sequence thereof,
d. at least one SNP selected from A at position 4508, A at position 11697, C at position 12229, A at position 13135, C at position 32979, G at position 38081, C at position 38220 of SEQ ID NO:87 or a variant, portion or complementary sequence thereof,
e. at least one SNP selected from A at position 30913, C at position 34950, C at position 38409, A at position 38458, T at position 42427, G at position 42604, T at position 43158 of SEQ ID NO:88 or a variant, portion or complementary sequence thereof,
f. SNP of G at position 1032 of SEQ ID NO:89 or a variant, portion or complementary sequence thereof,
g. SNP of A at position 173977 and/or SNP of T at position 228195 of SEQ ID NO:90 or a variant, portion or complementary sequence thereof, and
h. at least one SNP selected from C at position 857, T at position 15689, C at position 40135, G at position 49492 of SEQ ID NO:91 or a variant, portion or complementary sequence thereof.

According to a further embodiment, the present invention provides the use as defined in any of the above, wherein said variant comprises at least 90% sequence similarity to the corresponding SEQ ID nucleotide sequence.

According to a further embodiment, the present invention provides the use as defined in any of the above, wherein at least 5.5%, such as at least 10%, 20%, 30%, 40%, 50%, 60%, 70%, 80%, at least 90% or 100% of said P. persimilis predatory individuals of said population comprise said at least one SNP marker.

According to a further embodiment, the present invention provides the use as defined in any of the above, wherein the rearing population of P. persimilis predatory mite individuals is characterized by a daily reproduction rate (λ) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey.

According to a further embodiment, the present invention provides use of a nucleic acid sequence selected from SEQ ID NO:1 to SEQ ID NO:28 or a portion thereof to identify Phytoseiulus persimilis (P. persimilis) predatory mite individuals characterized by improved rearing properties on a non-Tetranychid arthropod prey as a factitious host, preferably on an immobilized non-Tetranychid arthropod prey, such as on an Astigmatid prey, most preferably on an immobilized Astigmatid prey, as compared to Phytoseiulus persimilis (P. persimilis) predatory mite individuals lacking the at least one sequence.

According to a further embodiment, the present invention provides the use as defined in any of the above, wherein the improved rearing properties on a non-Tetranychid arthropod prey are selected from the following parameters: increased daily reproduction rate (λ), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals lacking the at least one SNP and or the polynucleotide sequence selected from SEQ ID NO:1 to SEQ ID NO:28.

According to a further embodiment, the present invention provides an isolated nucleic acid primer pair sequence comprising at least 90% similarity to a polynucleotide sequence selected from SEQ ID NO:29 to SEQ ID NO:83 and any combination thereof.

According to a further embodiment, the present invention provides use of the isolated nucleic acid primer pair sequence as defined above, for identifying and/or selecting and/or obtaining Phytoseiulus persimilis (P. persimilis) predatory mite individual characterized by improved rearing properties on a non-Tetranychid arthropod prey as a factitious host, preferably on an immobilized non-Tetranychid arthropod prey, such as on an Astigmatid prey, most preferably on an immobilized Astigmatid prey, and/or for obtaining the Phytoseiulus persimilis (P. persimilis) predatory mite population as defined in any of the above, or mite individual as defined in any of the above.

Descriptions of embodiments of the invention in the present application are provided by way of example and are not intended to limit the scope of the invention. The described embodiments comprise different features, not all of which are required in all embodiments. Some embodiments utilize only some of the features or possible combinations of the features. Variations of embodiments of the invention that are described, and embodiments comprising different combinations of features noted in the described embodiments, will occur to a person having ordinary skill in the art. Unless explicitly stated, the method embodiments described herein are not constrained to a particular order in time or chronological sequence. Additionally, some of the described method elements may be skipped, or they may be repeated, during a sequence of operations of a method.

In order to understand the invention and to see how it may be implemented in practice, a plurality of preferred embodiments will now be described, by way of non-limiting example only, with reference to the following examples.

### Example 1

Identification of Genetic Markers Unique for P. persimilis Populations with the Trait of Improved Rearing Properties on a Non-Tetranychid Arthropod Prey

In this example, the genetic profile of P. persimilis population reared upon its natural Tetranychid prey (i.e. spider mites) (P−), was compared to the P. persimilis population of the present invention reared and reproduced on non-Tetranychid arthropod prey after selective breeding, preferably on Astigmatid prey such as C. lactis (P+).

### 1. Reference Genome Assembly:

A. Sample collection: For the purpose of assembling the genome (de novo genome assembly), it was aimed to obtain a population with low genetic variability. This was obtained by forcing sib mating for consecutive generations—Females originating from a P+ population were isolated and allowed to oviposit, then removed from their offspring. The offspring were allowed to mate with their siblings, and then an individual female was isolated to repeat the procedure. This was repeated for 15 generations. One resulting female of this process was the founder of a population that was maintained on frozen C. lactis as food for 9 months. A sample of this population was collected from the rearing.
B. Genomic DNA isolation protocol: The sampled population was subdivided to pools of ˜300 individuals. In total 3 pools were treated. Each pool was subjected to genomic DNA extraction. The QIAGEN DNeasy® Blood & Tissue Kit was used for DNA extraction according to manufacturer instructions with a modification: prior to incubation, samples were lysed using a mechanical homogenizer. Following extraction, an additional step of RNA degradation was done using ZYMO RESEARCH RNase A according to manufacturer instructions. Finally, extracts of the pools were joined and concentrated using the Genomic DNA Clean & Concentrator™-25 (ZYMO RESEARCH) according to manufacturer instructions. DNA quality and quantity were verified by automated electrophoresis, and Fluorescence-based quantification, respectively.
C. Genomic DNA library preparation and sequencing: Genomic DNA library preparation and sequencing were performed at GENEWIZ Inc. (NJ, USA). Illumina compatible paired-end library preparation and sequencing. Genomic DNA was fragmented and used for library preparation using Illumina Compatible Paired-End Sequencing kit. Libraries were sequenced on an Illumina HiSeq platform using High Output mode with a 2×150 PE sequencing configuration. In total, 81,229,004 pair-end reads were produced (24 Giga base of data). With mean quality score of 35.77 (Phred score scale).
D. Assembly of reference genome: SOAPdenovo v2.41, de-novo assembler, was used for de-novo assembly using the sequenced data as input. De-novo assembled genome was created with a minimum contig length of 1000 bp. QUAST was used to generate statistics for the de-novo assembled genome. The total length of the assembly was 167,708,956 bases (15,621 contigs; N50 of contig length was 68,160 bases). BUSCO was used to assess the assembly quality based on representation of an array of arthropods-typical single-copy genes (https://academic.oup.com/bioinformatics/article/31/19/3210/211866). BUSCO analysis indicated 72% completeness for the assembled genome.

### 2. Identification of P+ Specific Genetic Markers:

In order to identify genetic markers specific to P+ line, a variant calling strategy was applied as follows:
A. P. persimilis populations sampling: Sixteen different P. persimilis populations were sampled; three represented P+ line (One population that went through the sib-mating procedure described in section 1 above, and two P+ populations obtained as described in WO 20121/044404 incorporated herein by its entirety), and thirteen populations represented the traditional, conventionally reared P− line. The P− populations were not applied to the P+ breeding and selection processes, and represent a range of different commercial producers/populations, and geographic origins. From each population, 100-300 specimens were collected.
B. Genomic DNA extraction and high-throughput sequencing: Genomic DNA from each of the different populations was isolated in the method described in section 1b with the exception that no pooling of DNA from multiple sub samples was required. Library preparation and sequencing was done using the same methodology described in section 1c. In total, 2,895 million pair-end reads were sequenced (868 Giga base of data) with average read pairs per sample of 193 million. This sequencing depth corresponds to an ≥300 average coverage of the assembled genome. This allows for detailed depiction of genetic variants at the population level.
C. Variant calling: Data analysis for SNP/INDEL was performed using DRAGEN pipeline (https://genomemedicine.biomedcentral.com/articles/10.1186/si3073-015-0221-8). The DRAGEN (Dynamic Read Analysis for GENomics) platform is based on the highly reconfigurable DRAGEN Bio-IT Processor. The DRAGEN platform provides hardware-accelerated implementations of genome pipeline algorithms, including variant calling. The DRAGEN platform is optimized to handle Illumina HiSeq High data to detect comprehensively and accurately genomic variants including single nucleotide variants (SNV) and large INDELs with an option for accelerated GATK (Genome Analysis Toolkit). The P+ assembled draft genome was used as reference input and the sixteen samples' data sets were mapped to it. Following, variants were detected and analyzed.
D. Selection of P+ genetic markers: Following mapping and variant calling, a set of criteria were used in order to select for genetic markers unique to the P+ populations line. Those included:
   high quality score for the base called in the position (QUAL>99), high coverage of the variant in all populations (n>50; N>700),
   zero occurrence of the reference allele in non-P+ populations (13 populations), i.e., the reference allele, representing P+ genome was not found even once in any of the other populations, and,
   the reference allele is the designated allele in all tested P+ populations (three populations). Based on these criteria, a set of 34 P+ genetic markers was defined.
      3. Listing of genetic markers specific to P. persimilis line (P+):

Based on the above, a list of genetic markers unique to the P+ line of P. persimilis was generated and provided herewith (see Tables 1-3). Table 1 lists the details of each of the 34 genetic markers unique to P+ line of P. persimilis in contig-level.

TABLE 1
List of unique genetic markers identified
for P+ line positioned in contig-level

| | | Genomic | | Marker |
|---|---|---|---|---|
| | Contig SEQ | marker | Position in | allele |
| Contig name | ID No. | name | contig | (P+) |
| PP534_contig_2906 | 84 | SNP1 | 34739 | G |
| PP534_contig_4004 | 85 | SNP2 | 105452 | C |
| PP534_contig_4004 | 85 | SNP3 | 109775 | G |
| PP534_contig_4004 | 85 | SNP4 | 115020 | A |
| PP534_contig_4004 | 85 | SNP5 | 115073 | T |
| PP534_contig_4004 | 85 | SNP6 | 125940 | C |
| PP534_contig_4004 | 85 | SNP7 | 131548 | G |
| PP534_contig_4004 | 85 | SNP8 | 131555 | G |
| PP534_contig_11764 | 86 | SNP9 | 99333 | A |
| PP534_contig_11764 | 86 | SNP10 | 99595 | G |
| PP534_contig_11764 | 86 | SNP11 | 99605 | T |
| PP534_contig_11764 | 86 | SNP12 | 115325 | A |
| PP534_contig_11764 | 86 | SNP13 | 115354 | C |
| PP534_contig_11839 | 87 | SNP14 | 4508 | A |
| PP534_contig_11839 | 87 | SNP15 | 11697 | A |
| PP534_contig_11839 | 87 | SNP16 | 12229 | C |
| PP534_contig_11839 | 87 | SNP17 | 13135 | A |
| PP534_contig_11839 | 87 | SNP18 | 32979 | C |
| PP534_contig_11839 | 87 | SNP19 | 38081 | G |
| PP534_contig_11839 | 87 | SNP20 | 38220 | C |
| PP534_contig_12109 | 88 | SNP21 | 30913 | A |
| PP534_contig_12109 | 88 | SNP22 | 34950 | C |
| PP534_contig_12109 | 88 | SNP23 | 38409 | C |
| PP534_contig_12109 | 88 | SNP24 | 38458 | A |
| PP534_contig_12109 | 88 | SNP25 | 42427 | T |
| PP534_contig_12109 | 88 | SNP26 | 42604 | G |
| PP534_contig_12109 | 88 | SNP27 | 43158 | T |
| PP534_contig_12116 | 89 | SNP28 | 1032 | G |
| PP534_contig_12321 | 90 | SNP29 | 173977 | A |
| PP534_contig_12321 | 90 | SNP30 | 228195 | T |
| PP534_contig_12532 | 91 | SNP31 | 857 | C |
| PP534_contig_12532 | 91 | SNP32 | 15689 | T |
| PP534_contig_12532 | 91 | SNP33 | 40135 | C |
| PP534_contig_12532 | 91 | SNP34 | 49492 | G |

Genomic regions (reference sequences) of P. persimilis P+ line containing the genomic marker sites: In order to enable accurate comparison of any given P. persimilis population, to the P+ line, genomic sequences corresponding to the regions containing the genomic markers discovered by the present invention, are provided herein. These sequences are derived from the draft genome assembly referred to in section 1. Genomic regions for P. persimilis reference sequence containing P+ line specific markers, are set forth in SEQ ID NO:1-28 (see Table 2).

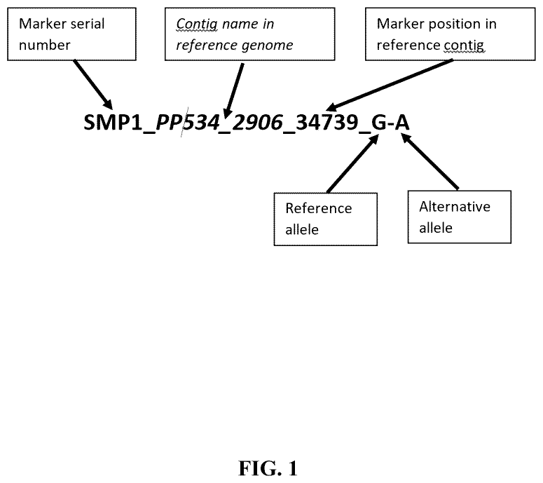

P+ line genomic regions containing markers nomenclature: Each genomic region containing a unique marker is denoted by a unique name that included the marker number (SNP01 to SNP34), the name of the contig in the reference genome assembly detailed in Table 1, the position of the marker along the contig (numerical value), and the reference and alternative (containing the unique P+ line SNP) allele bases. See an example of genomic region containing markers denomination presented in FIG. 1.

TABLE 2
List of unique genetic markers identified for P+ line in genomic regions- level

| | | Genomic | | Position | |
|---|---|---|---|---|---|
| Genomic | | region | Genomic | in | Marker |
| marker | | SEQ | region | Genomic | allele |
| name | Genomic region name | ID NO. | length | region | (P+) |
| SNP1 | SNP1_PP534_contig_2906_34739_G-A | SEQ ID | 1742 | 163 | G |
| | | NO: 1 | | | |
| SNP2 | SNP2_PP534_contig_4004_105452_C-T | SEQ ID | 778 | 181 | C |
| | | NO: 2 | | | |
| SNP3 | SNP3_PP534_contig_4004_109775_G-A | SEQ ID | 1818 | 197 | G |
| | | NO: 3 | | | |
| SNP4 | SNP4_PP534_contig_4004_115020_A-G \| | SEQ ID | 1560 | 111 | A |
| | SNP5_PP534_contig_4004_115073_T-C | NO: 4 | | | |
| SNP5 | SNP4_PP534_contig_4004_115020_A-G \| | SEQ ID | 1560 | 165 | T |
| | SNP5_PP534_contig_4004_115073_T-C | NO: 4 | | | |
| SNP6 | SNP6_PP534_contig_4004_125940_C-G | SEQ ID | 652 | 193 | C |
| | | NO: 5 | | | |
| SNP7 | SNP7_PP534_contig_4004_131548_G-A \| | SEQ ID | 968 | 186 | G |
| | SNP8_PP534_contig_4004_131555_G-A | NO: 6 | | | |
| SNP8 | SNP7_PP534_contig_4004_131548_G-A \| | SEQ ID | 968 | 193 | G |
| | SNP8_PP534_contig_4004_131555_G-A | NO: 6 | | | |
| SNP9 | SNP9_PP534_contig_11764_99333_A-G | SEQ ID | 533 | 161 | A |
| | | NO: 7 | | | |
| SNP10 | SNP10_PP534_contig_11764_99595_G-A \| | SEQ ID | 890 | 203 | G |
| | SNP11_PP534_contig_11764_99605_T-G | NO: 8 | | | |
| SNP11 | SNP10_PP534_contig_11764_99595_G-A \| | SEQ ID | 890 | 213 | T |
| | SNP11_PP534_contig_11764_99605_T-G | NO: 8 | | | |
| SNP12 | SNP12_PP534_contig_11764_115325_A-G \| | SEQ ID | 1652 | 163 | A |
| | SNP13_PP534_contig_11764_115354_C-T | NO: 9 | | | |
| SNP13 | SNP12_PP534_contig_11764_115325_A-G \| | SEQ ID | 1652 | 192 | C |
| | SNP13_PP534_contig_11764_115354_C-T | NO: 9 | | | |
| SNP14 | SNP14_PP534_contig_11839_4508_A-G | SEQ ID | 3251 | 208 | A |
| | | NO: 10 | | | |
| SNP15 | SNP15_PP534_contig_11839_11697_A-G | SEQ ID | 1541 | 199 | A |
| | | NO: 11 | | | |
| SNP16 | SNP16_PP534_contig_11839_12229_C-T | SEQ ID | 1076 | 266 | C |
| | | NO: 12 | | | |
| SNP17 | SNP17_PP534_contig_11839_13135_A-T | SEQ ID | 1495 | 216 | A |
| | | NO: 13 | | | |
| SNP18 | SNP18_PP534_contig_11839_32979_C-G | SEQ ID | 2397 | 187 | C |
| | | NO: 14 | | | |
| SNP19 | SNP19_PP534_contig_11839_38081_G-A | SEQ ID | 1565 | 279 | G |
| | | NO: 15 | | | |
| SNP20 | SNP20_PP534_contig_11839_38220_C-G | SEQ ID | 1402 | 188 | C |
| | | NO: 16 | | | |
| SNP21 | SNP21_PP534_contig_12109_30913_A-G | SEQ ID | 1704 | 165 | A |
| | | NO: 17 | | | |
| SNP22 | SNP22_PP534_contig_12109_34950_C-A | SEQ ID | 1938 | 183 | C |
| | | NO: 18 | | | |
| SNP23 | SNP23_PP534_contig_12109_38409_C-T \| | SEQ ID | 1471 | 133 | C |
| | SNP24_PP534_contig_12109_38458_A-T | NO: 19 | | | |
| SNP24 | SNP23_PP534_contig_12109_38409_C-T \| | SEQ ID | 1471 | 182 | A |
| | SNP24_PP534_contig_12109_38458_A-T | NO: 19 | | | |
| SNP25 | SNP25_PP534_contig_12109_42427_T-C \| | SEQ ID | 2196 | 146 | T |
| | SNP26_PP534_contig_12109_42604_G-A | NO: 20 | | | |
| SNP26 | SNP25_PP534_contig_12109_42427_T-C \| | SEQ ID | 2196 | 323 | G |
| | SNP26_PP534_contig_12109_42604_G-A | NO: 20 | | | |
| SNP27 | SNP27_PP534_contig_12109_43158_T-G | SEQ ID | 2298 | 241 | T |
| | | NO: 21 | | | |
| SNP28 | SNP28_PP534_contig_12116_1032_G-A | SEQ ID | 1941 | 236 | G |
| | | NO: 22 | | | |
| SNP29 | SNP29_PP534_contig_12321_173977_A-C | SEQ ID | 2018 | 199 | A |
| | | NO: 23 | | | |
| SNP30 | SNP30_PP534_contig_12321_228195_T-C | SEQ ID | 1779 | 185 | T |
| | | NO: 24 | | | |
| SNP31 | SNP31_PP534_contig_12532_857_C-G | SEQ ID | 2654 | 158 | C |
| | | NO: 25 | | | |
| SNP32 | SNP32_PP534_contig_12532_15689_T-C | SEQ ID | 2094 | 143 | T |
| | | NO: 26 | | | |
| SNP33 | SNP33_PP534_contig_12532_40135_C-T | SEQ ID | 2280 | 157 | C |
| | | NO: 27 | | | |
| SNP34 | SNP34_PP534_contig_12532_49492_G-A | SEQ ID | 1932 | 161 | G |
| | | NO: 28 | | | |

### 4. Assay for High-Throughput Sequencing-Based Verification:

An assay that enables the identification of alleles for the genetic markers in P. persimilis population is here detailed. This assay was calibrated and is relevant to population level assessment of allele status using samples of specimens that are equal or greater than 100 individuals.
A. Sample collection: Living samples of P. persimilis populations should be collected and stored in clean, dry and sealed receptacle, such as an Eppendorf tube. Samples should be either processed immediately or kept frozen at −70° C. or below until processing. Any treatment or handling of the sample that may damage the integrity of the sample and, specifically of DNA should be avoided. These include but are not limited to heating, UV irradiation, and emersion in bioactive liquids among other.
B. Genomic DNA isolation: The protocol specified in section 1b should be followed. The minimal quantity of genomic DNA required for continuation of the assay is 500 ng in total, as measured by fluorescence-based quantification (e.g., QuBit 3.0 instrument, ds-DNA assay kit), but preferably quantity would be above 1 μg of genomic DNA. The quality of genomic DNA extract should be tested by automated electrophoresis (e.g., TapeStation system, Agilent, CA, USA) and verified to be above DIN value of 8.0.
C. Genomic DNA library preparation and sequencing: Genomic DNA library should be prepared (e.g. using Illumina-compatible library preparation kits). Libraries quality should be verified before sequencing. Sequencing of ≥20 Giga bases of sequence per population (e.g. enabled by Illumina sequencing platform), allowing for ˜ 100 fold average coverage of the genome (based on an estimate of 160-180 M bp genome size) should be performed. Quality of the resulting sequences should be verified (e.g., by FastqC analysis).
D. Determination of allele status: The sequence data presented in Table 1 (SEQ ID NO:84-91) should be used as a reference for processing. Sequence data obtained for the tested population should be mapped to this reference sequence using designated tools such as Bowtie2 (https://www.nature.com/articles/nmeth.1923) or HISAT (https://www.nature.com/articles/nmeth.3317 #:˜:text=HISAT %20(hierarchical%20indexing%20for%20spliced,reads %20from%20RNA%20sequencing%20experiments). Mapping results should be formatted and viewed and inspected using a genome viewer such as IGV (https://cancerres.aacrjournals.org/content/77/21/e31.long). Variant statistics can be achieved automatically using GATK tools (https://www.biorxiv.org/content/10.1101/201178v3).

### 5. Assay for Sanger Sequencing-Based Verification:

An assay that enables the identification of alleles of the genetic markers in P. persimilis population is here detailed. This assay was calibrated and is relevant for individual specimen level assessment of allele status. In general, in the assay, DNA of individual mites from different populations was amplified by PCR using the specific primers for each marker. The product was sequenced and compared to the reference genome to assure the predicted SNPs are found.
A. Sample collection: Living samples of P. persimilis populations should be collected and stored in clean, dry and sealed receptacle, such as an Eppendorf tube. Samples should be either processed immediately or kept frozen at −70° C. or below until processing. Any treatment or handling of the sample that may damage the integrity of the sample and, specifically of DNA should be avoided. These include but are not limited to heating, UV irradiation, and emersion in bioactive liquids among other.
B. Genomic DNA isolation: Mites were squashed in 35 μl lysis buffer (5 mM Tris pH8, 0.5 mM EDTA pH8, 0.5% IGEPAL, 1 mg Proteinase K) and incubated at 65° C. for 15 minutes followed by 95° C. for 10 minutes.
C. Polymerase change reaction (PCR) amplification of target regions and sequencing: A set of PCR primers was designed in order to enable amplification of genomic regions containing a single or multiple sites of P+ line genomic markers. Table 3 contains the list of targets and respective primer pairs and their specifications (within the table, F denotes forward primer and R denotes reverse primer). PCR amplification was conducted using amplification enzyme (polymerase) with High-fidelity properties, such as Takara PrimeSTAR Max DNA Polymerase. Following successful amplification of specific fragments of denoted size, amplicons were sequenced using the Sanger methodology. Resulting sequences were inspected for quality.
D. Determination of allele status: Resulting high-quality sequences have been aligned to the reference sequences listed in Table 2 (SEQ ID NO:1-28). Following alignment, allele status have been determined by inspecting the nucleotide sequence at the position of the genomic marker. It was found that the genetic markers unique to P+ line were verified as such within individual specimens. In other words, alleles containing P+ specific genomic markers (i.e. SEQ ID NO:1-28) were verified at the single mite level of P. persimilis P+ populations. These markers were not found in non-P+P. persimilis populations (P− populations).

TABLE 3
List of primer pairs for the amplification by polymerase chain reaction of
genomic regions containing P+ line specific genomic markers

| | | | | Product |
|---|---|---|---|---|
| Primer | | | Template | length |
| pair name | Primer SEQ ID NO. and Sequence (5′→3′) | | strand | (bp) |
| SNP_1 | F | CCCTGACCATCGGTTCTCAC | Plus | 1718 |
| | SEQ ID NO: 29 | | | |
| | R | TATCTCTTTCAGTCAGTTCCTATGC | Minus | |
| | SEQ ID NO: 30 | | | |
| SNP_2 | F | GTCTAACACAGAGCGGGCAT | Plus | 759 |
| | SEQ ID NO: 31 | | | |
| | R | GTGGAAACTCCGATGCTGGA | Minus | |
| | SEQ ID NO: 32 | | | |
| SNP_3 | F | GAACGAGGCTCCGTGTACTT | Plus | 1799 |
| | SEQ ID NO: 33 | | | |
| | R | TCTGTGTTTACCTGCCCGTC | Minus | |
| | SEQ ID NO: 34 | | | |
| SNP_4-5 | F | AGGTGTCCGCCAATGGAATC | Plus | 1541 |
| | SEQ ID NO: 35 | | | |
| | R | ACGGGAGACTTGGGCTATCT | Minus | |
| | SEQ ID NO: 36 | | | |
| SNP_6 | F | ACACGGAACCATCCCATCAG | Plus | 633 |
| | SEQ ID NO: 37 | | | |
| | R | TGGAAGAAAGTCAGGTCGCC | Minus | |
| | SEQ ID NO: 38 | | | |
| SNP_7-8 | F | ACTCCTGGTCCCTGTCTCTC | Plus | 949 |
| | SEQ ID NO: 39 | | | |
| | R | GCTGAATAGCCGCTCCGTAT | Minus | |
| | SEQ ID NO: 40 | | | |
| SNP_9 | F | AACGCTTCCAGACCCTCATC | Plus | 514 |
| | SEQ ID NO: 41 | | | |
| | R | TTTCGGAAGAACGCGACAGA | Minus | |
| | SEQ ID NO: 42 | | | |
| SNP_10-11 | F | TCACTTCACTGTGCTGAGGC | Plus | 871 |
| | SEQ ID NO: 43 | | | |
| | R | CGCTAGTTTTGCATCCTCGC | Minus | |
| | SEQ ID NO: 44 | | | |
| SNP_12-13 | F | GCGGAGAGAGCTGGAATCTC | Plus | 1633 |
| | SEQ ID NO: 45 | | | |
| | R | TGCAGTCTCGAAGCGAAGTT | Minus | |
| | SEQ ID NO: 46 | | | |
| SNP_14 | F | AGTCCCCTTTACTACCCGCT | Plus | 3232 |
| | SEQ ID NO: 47 | | | |
| | R | TGAAGTTTCGCAGGTTCGTG | Minus | |
| | SEQ ID NO: 48 | | | |
| SNP_15-16 | F | AACGAAGCTAGATCGGCTGG | Plus | 1522 |
| | SEQ ID NO: 49 | | | |
| | R | CGGCTGGCATTTTGGCTATC | Minus | |
| | SEQ ID NO: 50 | | | |
| SNP_16 | F | CGAGCATTGGGTGAGAGCAT | Plus | 1057 |
| | SEQ ID NO: 51 | | | |
| | R | CGGCTGGCATTTTGGCTATC | Minus | |
| | SEQ ID NO: 50 | | | |
| SNP_17 | F | CGAAGTGAGCCCGATCCAAA | Plus | 1476 |
| | SEQ ID NO: 52 | | | |
| | R | TCAAACTCCCCCTCGTTTCG | Minus | |
| | SEQ ID NO: 53 | | | |
| SNP_18 | F | TTAGTCTACGGCTGCTCACG | Plus | 2378 |
| | SEQ ID NO: 54 | | | |
| | R | CCATACTCTTCGCTCGCCAT | Minus | |
| | SEQ ID NO: 55 | | | |
| SNP_19 | F | GCTCGGGAGTTCGAGCATAC | Plus | 1546 |
| | SEQ ID NO: 56 | | | |
| | R | TGCGTTTGAGTGGAATCCGA | Minus | |
| | SEQ ID NO: 57 | | | |
| SNP_20 | F | TCACGGTGCGACTTCCATTC | Plus | 1383 |
| | SEQ ID NO: 58 | | | |
| | R | TCCAAAACCGTCGCTCAAGA | Minus | |
| | SEQ ID NO: 59 | | | |
| SNP_21 | F | CTCGACAAAGTGCCGTCTGA | Plus | 1685 |
| | SEQ ID NO: 60 | | | |
| | R | AGCAATAAGGGGGACACAGC | Minus | |
| | SEQ ID NO: 61 | | | |
| SNP_22 | F | GCTCATGTCGAGGCTCTCTC | Plus | 1919 |
| | SEQ ID NO: 62 | | | |
| | R | ATAGGGTTCGGAGTTCGGGA | Minus | |
| | SEQ ID NO: 63 | | | |
| SNP_23-24 | F | GTGTGGTTGTCACAGGGGAT | Plus | 1452 |
| | SEQ ID NO: 64 | | | |
| | R | CTAGGTGAGTCGATGGCGTC | Minus | |
| | SEQ ID NO: 65 | | | |
| SNP_25-26 | F | GGCGATACAGACAACGCTCT | Plus | 2177 |
| | SEQ ID NO: 66 | | | |
| | R | GTACCCGCGAATCGTAGGAG | Minus | |
| | SEQ ID NO: 67 | | | |
| SNP_27 | F | CTGTCGCACACGAGGAGATA | Plus | 2279 |
| | SEQ ID NO: 68 | | | |
| | R | CTCAGAGGTTGCTGGAAGGG | Minus | |
| | SEQ ID NO: 69 | | | |
| SNP_28 | F | GGTGCTCGCTTTCGCTATTG | Plus | 1922 |
| | SEQ ID NO: 70 | | | |
| | R | TTTCACGGGGCTACATTGCT | Minus | |
| | SEQ ID NO: 71 | | | |
| SNP_29 | F | TCGCCTGAGAGAGGTACTGG | Plus | 1999 |
| | SEQ ID NO: 72 | | | |
| | R | GAGGGACGCAGTGGAATCAA | Minus | |
| | SEQ ID NO: 73 | | | |
| SNP_30 | F | CTGGGATTCGCTGCTCTGTT | Plus | 1760 |
| | SEQ ID NO: 74 | | | |
| | R | TTCTCAGAGGCGCTCGATTC | Minus | |
| | SEQ ID NO: 75 | | | |
| SNP_31 | F | ACTCGGCGAATCCTACGAAC | Plus | 2635 |
| | SEQ ID NO: 76 | | | |
| | R | ACAAGGTGCCGGAATCACAT | Minus | |
| | SEQ ID NO: 77 | | | |
| SNP_32 | F | TAGCATAACTGACCAGCCGC | Plus | 2075 |
| | SEQ ID NO: 78 | | | |
| | R | TCTCGGCTAATCCGCATGAC | Minus | |
| | SEQ ID NO: 79 | | | |
| SNP_33 | F | GCAGCCAGATATACCGCGAA | Plus | 2261 |
| | SEQ ID NO: 80 | | | |
| | R | TTGCGAGTTTTTCCACAGCG | Minus | |
| | SEQ ID NO: 81 | | | |
| SNP_34 | F | GGTCACGCCGAATTTGTGTC | Plus | 1913 |
| | SEQ ID NO: 82 | | | |
| | R | GAGGCAGACCAACTGGAGAC | Minus | |
| | SEQ ID NO: 83 | | | |

As shown above, the newly identified genetic markers of the present invention (SNP1-34) are highly informative, e.g. according to the polymorphism information content (PIC) index, and/or unique for P+P. persimilis populations/individual mites of the present invention, characterized by improved rearing properties on a non-Tetranychid arthropod prey, e.g. Astigmata prey species, preferably immobilized Astigmata prey species, such as immobilized C. lactis.

It is herein demonstrated that the genetic markers and sequences provided by the present invention (SEQ ID NO:1-91) are useful for producing, identifying and selecting P. persimilis predatory mites that have the advantageous trait of effectively developing and reproducing on a non-Tetranychid arthropod prey (e.g. an immobilized Astigmata prey species), over P. persimilis absent of these genetic markers, traditionally grown on its natural Tetranychid prey (spider mites).

In addition, it is shown by the present invention that the protocol herein established for the first time, for identifying the novel and unique genomic markers within P. persimilis (P+) genome, is robust and that the resulted genomic markers and sequences identified via this protocol are also robust and can identify and select for P. persimilis predatory mites that have the advantageous trait of effectively developing and reproducing a on non-Tetranychid arthropod prey, such as an Astigmata prey species.

The genetic profiling results of the present invention summarized in Tables 1 and 2 (and primer sequences for identifying the genetic markers in Table 3) present newly identified haplotypes comprising combinations of genetic variants depicted in these tables. The haplotype combinations are unique for and associated with the P+P. persimilis population type. The newly identified genetic variants can be used to produce, identify, detect, select and/or screen for the P+ population which exhibits significantly improved development and reproduction parameters preying on Astigmatid species, as compared with non-P+ population (P− population), which is absent of this allelic haplotype combination. These reproduction parameters may include increased reproduction rate, increased oviposition rate and high percentage of female individuals capable of development and reproduction on the non-Tetranychid arthropod prey, and enhanced juvenile and adult female survival rate. The selected P. persimilis population of the present invention exhibits a similar and even improved ability to control their natural prey, spider mites.

To conclude, the present invention provides for the first time genomic biomarkers associated with the trait of increased reproduction rate of P. persimilis population on an alternative diet of non-Tetranychid arthropod prey, preferably Astigmatid mites such as C. lactis. This enables the production and identification of P. persimilis predatory mites characterized by a highly desirable, revolutionary, indoor rearing, exhibiting increased yield, feeding on Astigmata species.

### Example 2

Validation of Identified Genetic Markers Associated with P. persimilis Populations Characterized by Improved Rearing Properties on Non-Tetranychid Arthropod Prey

To validate the genetic markers (SNPs) defined in Example 1, different P. persimilis populations were sampled and tested for their genetic profile using the two methods described herein above—either by population-wide high-throughput genomic DNA sequencing for all of the markers, performed on a pool from each population, or by testing individual mites from different populations by sequencing the PCR product of a selected marker. Three validations (separated by seven and six months from each other) were conducted for the population-wide high-throughput genomic DNA sequencing method, and two validations (separated by 10 months from each other), were conducted for the PCR method.

### Validation by Population-Wide High-Throughput Genomic DNA Sequencing (Population Level)

Reference is now made to Table 4, presenting rate of P+ allele in the different P. persimilis populations tested. P. persimilis populations of P+, Alternation, and P− (including ‘other producer’ population) were sampled (sampling events 1, 2 and 3). Each sample consisted of 300 individual specimens. Total DNA was extracted, sequenced by High-throughput sequencing (population-wide high-throughput genomic DNA sequencing method), and mapped to the P+ reference genome assembly. Percentage of alleles in the population that are identical to P+ were calculated and presented for the 34 P+ unique SNPs (SNP1-34, see Tables 1 and 2).

TABLE 4
Rate of P+ allele in P+, Alternation, and P− P. persimilis populations using the
population-wide high-throughput genomic DNA sequencing method
producer

| | Line | | | | | | | | | | |
|---|---|---|---|---|---|---|---|---|---|---|---|
| | | | | | | | | | | | Other |
| Sampling | P+ | | | Alternation | | P− | | | | | producer |
| event | 1 | 2 | 3 | 2 | 3 | 1 | 1 | 1 | 2 | 3 | 1 |
| SNP1 | 85.71 | 85.09 | 100 | 83.10811 | 80.00 | 1.54 | 0 | 4.94 | 1.15 | 5.15 | 4.35 |
| SNP2 | 100 | 100 | 100 | 60.80 | 73.91 | 0 | 0 | 0 | 0.68 | 0 | 1.43 |
| SNP3 | 100 | 100 | 100 | 67.02 | 82.29 | 1.67 | 0 | 0 | 1.55 | 0 | 0 |
| SNP4 | 100 | 100 | 100 | 54.73 | 81.16 | 0 | 0 | 0 | 0.66 | 0 | 0 |
| SNP5 | 100 | 100 | 100 | 58.82 | 84.00 | 0 | 0 | 0 | 0 | 0 | 0 |
| SNP6 | 89.86 | 91.34 | 100 | 66.87 | 70.45 | 0 | 0 | 0 | 0.65 | 0 | 0 |
| SNP7 | 100 | 100 | 100 | 79.41 | 80.95 | 1.47 | 1.56 | 0 | 0.79 | 0 | 0 |
| SNP8 | 100 | 100 | 100 | 78.20 | 78.05 | 0 | 0 | 0 | 0.78 | 0 | 0 |
| SNP9 | 100 | 89.23 | 100 | 85.71 | 79.71 | 0 | 0 | 0 | 0 | 0 | 1.22 |
| SNP10 | 88.71 | 100 | 91.38 | 84.75 | 60.47 | 0 | 0 | 0 | 0 | 0 | 3.03 |
| SNP11 | 86.76 | 100 | 91.67 | 85.95 | 62.79 | 0 | 0 | 0 | 0 | 0 | 0 |
| SNP12 | 80.30 | 100 | 87.63 | 81.82 | 79.41 | 0 | 0 | 1.41 | 0 | 0 | 0 |
| SNP13 | 80.56 | 100 | 86.60 | 80.31 | 81.48 | 0 | 0 | 1.23 | 0 | 0 | 0 |
| SNP14 | 100 | 100 | 100 | 86.32 | 100 | 0 | 0 | 0 | 0.71 | 0 | 0 |
| SNP15 | 100 | 100 | 100 | 88.97 | 100 | 0 | 0 | 0 | 0.67 | 0.93 | 0 |
| SNP16 | 100 | 100 | 100 | 88.24 | 100 | 0 | 0 | 0 | 2.22 | 0 | 0 |
| SNP17 | 100 | 100 | 100 | 100 | 100 | 0 | 0 | 0 | 1.56 | 0 | 0 |
| SNP18 | 100 | 89.36 | 100 | 83.46 | 86.46 | 0 | 0 | 0 | 1.04 | 0 | 0 |
| SNP19 | 100 | 100 | 100 | 89.23 | 86.17 | 0 | 0 | 0 | 0 | 0 | 1.52 |
| SNP20 | 100 | 100 | 100 | 85.35 | 100 | 0 | 1.61 | 0 | 1.33 | 0 | 1.01 |
| SNP21 | 100 | 100 | 100 | 80.95 | 85.71 | 0 | 0 | 0 | 2.54 | 0 | 0 |
| SNP22 | 100 | 100 | 100 | 76.00 | 68.75 | 4.08 | 0 | 0 | 2.48 | 0.85 | 0 |
| SNP23 | 100 | 100 | 100 | 80.67 | 74.03 | 0 | 0 | 0 | 1.92 | 1.89 | 1.32 |
| SNP24 | 100 | 100 | 100 | 77.37 | 79.73 | 0 | 0 | 0 | 1.72 | 1.96 | 1.19 |
| SNP25 | 100 | 100 | 100 | 74.24 | 83.00 | 0 | 0 | 0 | 3.55 | 1.63 | 0 |
| SNP26 | 100 | 100 | 100 | 74.10 | 87.39 | 0 | 0 | 1.47 | 3.27 | 0 | 2.30 |
| SNP27 | 100 | 100 | 100 | 78.46 | 86.52 | 0 | 0 | 0 | 0.79 | 0 | 3.33 |
| SNP28 | 100 | 100 | 100 | 87.50 | 84.88 | 2.38 | 0 | 1.02 | 1.59 | 0 | 1.19 |
| SNP29 | 80.52 | 81.48 | 87.32 | 62.20 | 67.35 | 0 | 0 | 0 | 0.81 | 0 | 0 |
| SNP30 | 79.49 | 78.29 | 84.34 | 66.00 | 57.53 | 0 | 1.72 | 0 | 1.68 | 0 | 0 |
| SNP31 | 100 | 100 | 100 | 76.71 | 74.36 | 0 | 1.59 | 0 | 0.76 | 0 | 0 |
| SNP32 | 100 | 100 | 100 | 67.97 | 75.29 | 0 | 0 | 0 | 1.32 | 0 | 0 |
| SNP33 | 100 | 100 | 100 | 75.00 | 83.33 | 0 | 0 | 0 | 2.61 | 0 | 1.09 |
| SNP34 | 100 | 85.95 | 100 | 74.15 | 80.99 | 0 | 0 | 0 | 0.69 | 0 | 0 |
| Max % ref | 100 | 100 | 100 | 100 | 100 | 4.08 | 1.72 | 4.94 | 3.55 | 5.15 | 4.35 |

As can be seen, the maximum percentage of identity to the P+ reference allele, in the P− population (incl. other producer P− population), was 5.15% (SNP1). While, the minimum percentage of identity to the P+ reference allele in the P+ examined populations was 80.30% (SNP12). This significant and clear difference shows the uniqueness of the SNPs found by the inventors to the P+ type population, and that they are highly informative and can effectively differentiate between P+ (having the trait of improved rearing on non-Tetranychid arthropod prey) and P− (lacking the trait) populations. With respect to the Alternation population, the minimum identity to the P+ reference allele was 60.47 (SNP10). This value is closer to the P+ type population, and shows that although percentage of the unique SNPs is slightly lower, this population, exposed to alternation of food source, encompass and exhibits the trait of improved rearing on non-Tetranychid arthropod prey, associated with the herein disclosed haplotype of SNPs.

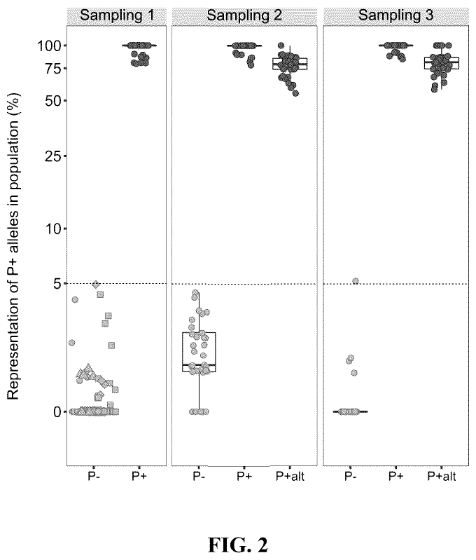

Reference is now made to FIG. 2, graphically presenting the rate of P+ allele in P. persimilis populations. Populations of P+ and P− (incl. other producer P− population) P. persimilis were sampled (sampling 1, 2 and 3). Each sample consisted of 300 individual specimens. Total DNA was extracted, sequenced by High-throughput sequencing (population-wide high-throughput genomic DNA sequencing method) and mapped to the P+ reference genome assembly. Each dot represents an allele (SNP). Dots of different symbols represent different populations at the same sampling date. Dotted line represents cutoff for identification as P+ (e.g. above 5%).

As can be clearly seen, the percentage of each of the P+ reference alleles (SNPs) in P− populations is about 5% or less, while the percentage of each of the P+ reference alleles in the P+ population was at least 80% or higher. In the Alternation population (P+ Alt) the percentage of the P+ allele was 60% or higher. These results demonstrate the correlation or association between the trait of improved rearing on non-Tetranychid arthropod prey (P+ population type) and each of the SNPs (alleles) herein described and any of their combination.

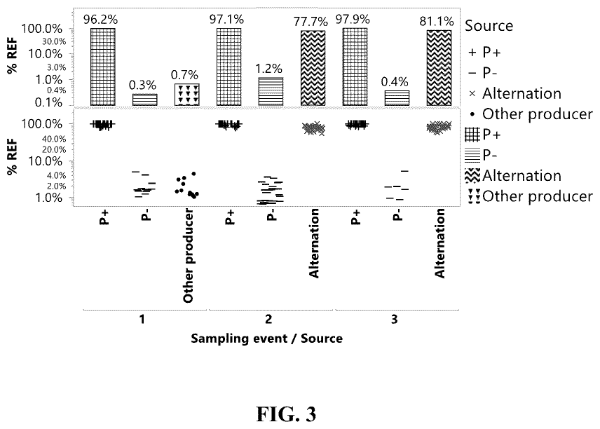

Reference is now made to FIG. 3, graphically alternatively presenting results of population-wide high-throughput genomic DNA sequencing of samples of different sources and different dates. In this graph, the Y axis shows the percentage of similarity to the defined P+ reference allele. The top graph part shows average results of all of the different markers tested, and in the bottom graph part, each dot represents the result for a specific marker for each sample. The X axis shows different sample times (events) and different sources.

As shown in FIG. 3, when populations were tested by population-wide high-throughput genomic DNA sequencing method, the P+ results showed almost 100% similarity to the reference allele, while P− populations (P− and ‘other producer’ sources) showed slightly higher than 0% similarity to the P+ reference allele (see FIG. 3).

With respect to a population maintained on an alternating diet (‘Alternation’ in FIG. 2, see also Example 3), it showed intermediate results, and was more similar or related to the P+ than to the P− population (about 810% similarity to the P+ reference allele, see FIG. 3).

### Validation by PCR (Individual Level)

When tested by amplifying the specific markers SNP2, SNP7 and SNP8, all P+ individuals matched the P+ alleles, while all of the P− individuals (incl. individuals sampled from ‘another producer’ source), did not match the corresponding the P+ alleles. The population maintained on an alternating diet showed, as expected, intermediate results, more closely related to the P+ population.

Reference is now made to Tables 5 and 6, presenting results of genetic similarity of different P. persimilis population types or sources to the P+ reference allele, as found by sequencing the PCR product of three different markers. Table 5 presents the percentage of mites exhibiting the same allele as the P+ reference from different sample events and different sources. Table 6 presents the number of mites individually tested from each group (sample event/source) and marker.

The results clearly show that 100% of the P+ population individuals tested has the P+ reference allele, while 0% of the tested P− population individuals has the P+ reference allele, for all three tested SNP markers. These data strengthen the informativeness and uniqueness of the genetic markers disclosed for the first time by the present invention, as associated with the P+ population trait of improved reproduction and rearing on a non-Tetranychid arthropod prey. The results obtained from individuals of the ‘Alternation’ population show that more than 60% (e.g. 60%-90%) of the tested individuals has the P+ reference allele. This implies that the ‘Alternation’ population is composed of individuals with the trait of improved rearing on a non-Tetranychid arthropod prey, and individuals absent of the trait (see Example 3).

TABLE 5
Percentage of mites exhibiting the P+ reference allele from different sample
events and different sources using the PCR method

| | Sampling event 1 | | | Sampling event 2 | | |
|---|---|---|---|---|---|---|
| Source | SNP2 | SNP7 | SNP8 | SNP2 | SNP7 | SNP8 |
| P+ | 100% | 100% | 100% | 100% | 100% | 100% |
| P− | 0% | 0% | 0% | 0% | 0% | 0% |
| Alternation | . | . | . | 67% | 90% | 90% |
| Other | 0% | 0% | . | . | . | . |
| producer | | | | | | |

TABLE 6
Number of mites individually tested for P+ reference allele from each sampling
event/source group and each marker using the PCR method

| | Sampling event 1 | | | Sampling event 2 | | |
|---|---|---|---|---|---|---|
| Source ↓ | SNP2 | SNP7 | SNP8 | SNP2 | SNP7 | SNP8 |
| P+ | 17 | 28 | 8 | 15 | 14 | 14 |
| P− | 18 | 6 | 4 | 15 | 14 | 14 |
| Alternation | 0 | 0 | 0 | 15 | 10 | 10 |
| Other | 11 | 8 | 0 | 0 | 0 | 0 |
| producer | | | | | | |

### Example 3

Rearing Success of a P. persimilis Population with Alternating Diet Between Carpoglyphus lactis (C. lactis) and Tetranychus urticae (T. urticae).

A P+ population has been reared on an alternating diet—C. lactis (as an exemplified Astigmatid prey mite species) for three months, and T. urticae for three months—these feeding regimes repeatedly alternated for many cycles. This procedure has been maintained for 4 years, after which the population's ability to be reared on C. lactis was quantified two times in comparison with the P+ populations (continuously reared on an Astigmatid prey mite species, such as C. lactis). The rearing of each population started from 1,000 individuals, and the total population was estimated 6 weeks later. A mixture from each population was weighed, and four samples containing about 50 mg each were taken, placed on a black adhesive tape and counted. Total population size was calculated according to these counts. The multiplication factor was calculated by dividing the total number of the individuals found, by 1000, giving the factor by which the population multiplied during the trial. To calculate the daily multiplication rate, Lambda, the 42^{nd} root of this calculated number was taken according to the following formula:

$$λ = \sqrt[t]{\frac{N ( t}{N ( 0 )}}$$

where λ is the daily multiplication rate, N(0) is the initial number of mites left for rearing (i.e. 1000 mites), N(t) is the total number of mites found after rearing for a 6 weeks period of time, and t=42.

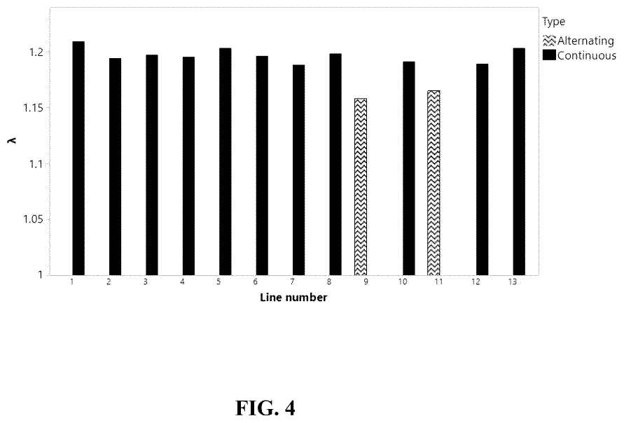

Reference is now made to FIG. 4 graphically presenting Lambda values of different P+‘continuous’ and ‘Alternating’ population lines. As shown in FIG. 4, the results of the ‘Alternating’ population were lower than those maintained on C. lactis continuously (i.e. P+‘continuous’ populations), demonstrating that this population has both lower rearing/breeding capacity on C. lactis, and a lower genetic similarity to the P+ population (as shown above). The P− population, which is showed herein to have the lowest genetic similarity to P+ reference allele, possesses the lowest lambda values (not presented in the figure, demonstrated in WO2019171374 and WO2021044404 publications incorporated in their entirety).

The Lambda values and the genetic similarity to the P+ population of: (1) P− population, (2) the ‘Alternating’ population, and (3) the P+ population, are summarized in Table 7. In Table 7, the average Lambda values and the percentage of similarity to the P+ allele of 3 types of populations, namely, P+, P− and an ‘Alternating’ population, are presented.

TABLE 7
Average Lambda values and percentage of similarity to the P+ allele
of the P+, P− and ‘Alternating’ populations

| | | Percentage similarity |
|---|---|---|
| Population | Lambda | to reference P+ allele |
| P+ | 1.18 | 97.1% |
| Alternating | 1.16 | 84.3% |
| P− | 1.06 | 0.46% |

It can be seen from the table above that the higher the percentage of genetic similarity to the P+ allele was, the higher was the rearing success/rate on a non-Tetranychid arthropod prey, as reflected by the lambda value. This shows the positive correlation and association between the identified unique P+ alleles (at least one of genetic markers SNP 1-34) and the trait of P. persimilis improved rearing and reproduction on a non-Tetranychid arthropod prey.

### REFERENCES

McMurtry, J. A. & Croft, B. A. (1997). Life-styles of phytoseiid mites and their roles in biological control. Annual Review of Entomology, 42, 291-321.
MCMURTRY JAMES A., DE MORAES GILBERTO J. & SOURASSOU NAZER FAMAH (2013) Revision of the lifestyles of phytoseiid mites (Acari: Phytoseiidae) and implications for biological control strategies. Systematic & Applied Acarology 18(4): 297-320. http://dx.doi.org/10.11158/saa.18.4.1
van de Vrie, M., McMurtry J. A. & Huffaker C. B. (1972) Ecology of tetranychid mites and their natural enemies: A review: III. Biology, ecology, and pest status, and host-plant relations of tetranychids. Hilgardia 41(13):343-432.

1. A biological control composition comprising a cultivated rearing population of Phytoseiulus persimilis (P. persimilis) predatory mite individuals, and optionally a carrier material, wherein at least 5% of said P. persimilis individuals comprise within their genome at least one Single Nucleotide Polymorphism (SNP) marker selected from:
a. SNP at position 34739 of a sequence having at least 90% sequence similarity to SEQ ID NO:84 or complementary sequence thereof,
b. at least one SNP at a position selected from 105452, 109775, 115020, 115073, 125940, 131548 and 131555, of a sequence having at least 90% sequence similarity to SEQ ID NO:85 or complementary sequence thereof,
c. at least one SNP at a position selected from 99333, 99595, 99605, 115325 and 115354, of a sequence having at least 90% sequence similarity to SEQ ID NO:86 or complementary sequence thereof,
d. at least one SNP at a position selected from 4508, 11697, 12229, 13135, 32979, 38081, 38220, of a sequence having at least 90% sequence similarity to SEQ ID NO:87 r or complementary sequence thereof,
e. at least one SNP at a position selected from 30913, 34950, 38409, 38458, 42427, 42604 and 43158 of a sequence having at least 90% sequence similarity to SEQ ID NO:88 or a variant, portion or complementary sequence thereof,
f. SNP at position 1032 of a sequence having at least 90% sequence similarity to SEQ ID NO:89 or complementary sequence thereof,
g. SNP at position 173977 and/or SNP at position 228195 of a sequence having at least 90% sequence similarity to SEQ ID NO:90 or complementary sequence thereof, and
h. at least one SNP at a position selected from 857, 15689, 40135 and 49492 of a sequence having at least 90% sequence similarity to SEQ ID NO:91 or complementary sequence thereof.

2. The biological control composition according to claim 1, wherein the at least one SNP is selected from:
a. SNP of G at position 34739 of a sequence having at least 90% sequence similarity to SEQ ID NO:84 or complementary sequence thereof,
b. at least one SNP selected from C at position 105452, G at position 109775, A at position 115020, T at position 115073, C at position 125940, G at position 131548, G at position 131555, of a sequence having at least 90% sequence similarity to SEQ ID NO:85 or complementary sequence thereof,
c. at least one SNP selected from A at position 99333, G at position 99595, T at position 99605, A at position 115325, C at position 115354, of a sequence having at least 90% sequence similarity to SEQ ID NO:86 or complementary sequence thereof,
d. at least one SNP selected from A at position 4508, A at position 11697, C at position 12229, A at position 13135, C at position 32979, G at position 38081, C at position 38220 of a sequence having g at least 90% sequence similarity to SEQ ID NO:87 or complementary sequence thereof,
e. at least one SNP selected from A at position 30913, C at position 34950, C at position 38409, A at position 38458, T at position 42427, G at position 42604, T at position 43158 of a sequence having at least 90% sequence similarity to SEQ ID NO:88 or complementary sequence thereof,
f. SNP of G at position 1032 of a sequence having at least 90% sequence similarity to SEQ ID NO:89 or complementary sequence thereof,
g. SNP of A at position 173977 and/or SNP of T at position 228195 of a sequence having at least 90% sequence similarity to SEQ ID NO:90 or complementary sequence thereof, and
h. at least one SNP selected from C at position 857, T at position 15689, C at position 40135, G at position 49492 of a sequence having at least 90% sequence similarity to SEQ ID NO:91 or complementary sequence thereof.

3-5. (canceled)

6. The biological control composition according to claim 1, wherein the at least 5% of said P. persimilis predatory individuals comprise within their genome a polynucleotide genomic sequence selected from (a) at least one sequence selected from SEQ ID NO:1 to SEQ ID NO:28 and SEQ ID NO:84 to SEQ ID NO: SEQ ID NO:91, and any combination thereof.

7. The biological control composition according to claim 1, wherein the cultivated rearing population of P. persimilis predatory mite individuals is characterized by improved rearing properties on a non-Tetranychid arthropod prey as a factious host, preferably on an immobilized non-Tetranychid arthropod prey, such as on an Astigmatid prey, most preferably on an immobilized Astigmatid prey, as compared to a P. persimilis population characterized by less than 5% individuals comprising within their genome said at least one SNP marker.

8. The biological control composition according to claim 7, wherein the improved rearing properties of the cultivated rearing P. persimilis population on a non-Tetranychid arthropod prey, are selected from
a. a daily reproduction rate (A) of above 1.06, preferably in the range of 1.10-1.40, such as 1.15-1.40, 1.20-1.40, 1.25-1.40, 1.30-1.40, or 1.10-1.35, 1.10-1.30, 1.10-1.25, 1.10-1.20, while preying on a non-Tetranychid arthropod as a food source, preferably on an immobilized non-Tetranychid arthropod prey, such as on an Astigmatid prey, most preferably on an immobilized Astigmatid prey; and
b. at least one of: increased daily reproduction rate (A), increased daily oviposition rate, increased survival rate, lower mortality rate, increased juvenile and/or female survival rate, increased percentage of female individuals reproducing on said prey, and improved or similar predatory behavior towards a Tetranychid species, as compared to a population of P. persimilis individuals characterized by less than 5% individuals comprising within their genome said at least one SNP marker.

9. The biological control composition according to claim 1, wherein the composition further comprises as a food source for the P. persimilis predatory mites of the cultivated rearing population, a non-Tetranychid arthropod prey, preferably an immobilized non-Tetranychid arthropod prey, such as on an Astigmatid prey, most preferably on an immobilized Astigmatid prey comprising immobilized life stages comprising immobilized eggs.

10. The biological control composition according to claim 7, wherein the non Tetranychid non-Tertranychid arthropod prey is selected from an Astigmatid mite species, an Artemia species, species from the Phytoseiidae family, Thrips such as Frankliniella occidentalis, Tarsonemidae mite species, and lepidopteran eggs such as of Ephestia kunhiela.

11-19. (canceled)

20. A method for obtaining a biological control composition comprising a cultivated rearing population of Phytoseiulus persimilis (P. persimilis) predatory mite individuals according to claim 1, wherein said method comprises steps of:
a. providing a rearing population of a P. persimilis predatory mite species, the rearing population comprising individuals of the P. persimilis species, reared on a suitable food source for the Phytoseiulus individuals, said food source comprising a prey species selected from the Tetranychidae species;
b. providing a population of individuals of preselected non-Tetranychid arthropod species, preferably an Astigmatid mite species, most preferably an immobilized Astigmatid mite species having immobilized life stages comprising immobilized eggs;
c. rearing the P. persimilis individuals on the preselected non-Tetranychid arthropod species as a food source;
d. screening the genome of a sample of P. persimilis individuals for the presence of at least 5% of at least one SNP selected from:
i. SNP of G at position 34739 of SEQ ID NO:84 or a variant or complementary sequence thereof,
ii. at least one SNP selected from C at position 105452, G at position 109775, A at position 115020, T at position 115073, C at position 125940, G at position 131548, G at position 131555, of SEQ ID NO:85 or a variant or complementary sequence thereof,
iii. at least one SNP selected from A at position 99333, G at position 99595, T at position 99605, A at position 115325, C at position 115354, of SEQ ID NO:86 or a variant or complementary sequence thereof,
iv. at least one SNP selected from A at position 4508, A at position 11697, C at position 12229, A at position 13135, C at position 32979, G at position 38081, C at position 38220 of SEQ ID NO:87 or a variant or complementary sequence thereof,
v. at least one SNP selected from A at position 30913, C at position 34950, C at position 38409, A at position 38458, T at position 42427, G at position 42604, T at position 43158 of SEQ ID NO:88 or a variant or complementary sequence thereof,
vi. SNP of G at position 1032 of SEQ ID NO:89 or a variant or complementary sequence thereof,
vii. SNP of A at position 173977 and/or SNP of T at position 228195 of SEQ ID NO:90 or a variant or complementary sequence thereof, and
viii. at least one SNP selected from C at position 857, T at position 15689, C at position 40135, G at position 49492 of SEQ ID NO:91 or a variant or complementary sequence thereof;
e. optionally, alternatingly rearing selected Phytoseiulus individuals in a sequence of:
rearing for at least 2, such as between 5 and 50, generations while using a food source comprising the preselected non-Tetranychid arthropod species;
rearing for at least 2, such as between 5 and 50, generations while using a food source comprising the prey species selected from the Tetranychidae
screening the genome of a sample of P. persimilis individuals for the presence of at least 5% of the at least one SNP.

21-26. (canceled)

27. A method for screening for or identifying a biological control composition according to claim 1, the method comprises steps of:
a. obtaining a sample of said population of individuals, the sample preferably comprise at least 50 individuals;
b. pooling the genome said individuals and screening the pooled genome for the presence of at least 5% of at least one SNP selected from:
i. SNP of G at position 34739 of SEQ ID NO:84 or a variant or complementary sequence thereof,
ii. at least one SNP selected from C at position 105452, G at position 109775, A at position 115020, T at position 115073, C at position 125940, G at position 131548, G at position 131555, of SEQ ID NO:85 or a variant or complementary sequence thereof,
iii. at least one SNP selected from A at position 99333, G at position 99595, T at position 99605, A at position 115325, C at position 115354, of SEQ ID NO:86 or a variant or complementary sequence thereof,
iv. at least one SNP selected from A at position 4508, A at position 11697, C at position 12229, A at position 13135, C at position 32979, G at position 38081, C at position 38220 of SEQ ID NO:87 or a variant or complementary sequence thereof,
v. at least one SNP selected from A at position 30913, C at position 34950, C at position 38409, A at position 38458, T at position 42427, G at position 42604, T at position 43158 of SEQ ID NO:88 or a variant or complementary sequence thereof,
vi. SNP of G at position 1032 of SEQ ID NO:89 or a variant or complementary sequence thereof,
vii. SNP of A at position 173977 and/or SNP of T at position 228195 of SEQ ID NO:90 or a variant or complementary sequence thereof, and
viii. at least one SNP selected from C at position 857, T at position 15689, C at position 40135, G at position 49492 of SEQ ID NO:91 or a variant or complementary sequence thereof.

28. A cultivated Phytoseiulus persimilis (P. persimilis) mite individual, wherein said cultivated Phytoseiulus predatory individual comprises within its genome at least one single nucleotide polymorphism (SNP) marker selected from:
a. SNP1 at position corresponding to position 163 of a sequence comprising at least 90% sequence similarity to SEQ ID NO:1 or complementary sequence thereof,
b. SNP2 at position 181 of a sequence having at least 90% sequence similarity to SEQ ID NO:2 or complementary sequence thereof,
c. SNP3 at position 197 of a sequence having at least 90% sequence similarity to SEQ ID NO:3 or complementary sequence thereof,
d. SNP4 at position 111 and/or SNP5 at position 165 of a sequence having at least 90% sequence similarity to SEQ ID NO:4 or complementary sequence thereof,
e. SNP6 at position 193 of a sequence having at least 90% sequence similarity to SEQ ID NO:5 or complementary sequence thereof,
f. SNP7 at position 186 and/or SNP8 at position 193 of a sequence having at least 90% sequence similarity to SEQ ID NO:6 or complementary sequence thereof,
g. SNP9 at position 161 of a sequence having at least 90% sequence similarity to SEQ ID NO:7 or complementary sequence thereof,
h. SNP10 at position 203 and/or SNP11 at position 213 of a sequence having at least 90% sequence similarity to SEQ ID NO:8 or complementary sequence thereof,
i. SNP12 at position 163 and/or SNP13 at position 192 of a sequence having at least 90% sequence similarity to SEQ ID NO:9 or complementary sequence thereof,
j. SNP14 at position 208 of a sequence having at least 90% sequence similarity to SEQ ID NO:10 or complementary sequence thereof,
k. SNP15 at position 199 of a sequence having at least 90% sequence similarity to SEQ ID NO:11 or complementary sequence thereof,
l. SNP16 at position 266 of a sequence having at least 90% sequence similarity to SEQ ID NO:12 or complementary sequence thereof,
m. SNP17 at position 216 of a sequence having at least 90% sequence similarity to SEQ ID NO:13 or complementary sequence thereof,
n. SNP18 at position 187 of a sequence having at least 90% sequence similarity to SEQ ID NO:14 or complementary sequence thereof,
o. SNP19 at position 279 of a sequence having at least 90% sequence similarity to SEQ ID NO:15 or complementary sequence thereof,
p. SNP20 at position 188 of a sequence having at least 90% sequence similarity to SEQ ID NO:16 or complementary sequence thereof,
q. SNP21 at position 165 of a sequence having at least 90% sequence similarity to SEQ ID NO:17 or complementary sequence thereof,
r. SNP22 at position 183 of a sequence having at least 90% sequence similarity to SEQ ID NO:18 or complementary sequence thereof,
s. SNP23 at position 133 and/or SNP24 at position 182 of a sequence having at least 90% sequence similarity to SEQ ID NO:19 or complementary sequence thereof,
t. SNP25 at position 146 and/or SNP26 position 323 of a sequence having at least 90% sequence similarity to SEQ ID NO:20 or complementary sequence thereof,
u. SNP27 at position 241 of a sequence having at least 90% sequence similarity to SEQ ID NO:21 or complementary sequence thereof,
v. SNP28 at position 236 of a sequence having at least 90% sequence similarity to SEQ ID NO:22 or complementary sequence thereof,
w. SNP29 at position 199 of a sequence having at least 90% sequence similarity to SEQ ID NO:23 or complementary sequence thereof,
x. SNP30 at position 185 of a sequence having at least 90% sequence similarity to SEQ ID NO:24 or complementary sequence thereof,
y. SNP31 at position 158 of a sequence having at least 90% sequence similarity to SEQ ID NO: or complementary sequence thereof,
z. SNP32 at position 143 of a sequence having at least 90% sequence similarity to SEQ ID NO:26 or complementary sequence thereof,
aa. SNP33 at position 157 of a sequence having at least 90% sequence similarity to SEQ ID NO:27 or complementary sequence thereof, and
bb. SNP34 at position 161 of a sequence having at least 90% sequence similarity to SEQ ID NO:28 or complementary sequence thereof, and any combination thereof.

29. (canceled)

30. The cultivated P. persimilis mite individual according to claim 28, wherein said P. persimilis predatory individual comprising within it's genome a polynucleotide sequence selected from SEQ ID NO:1 to SEQ ID NO:28, and any combination thereof.

31. The cultivated P. persimilis mite individual according to claim 28, wherein the at least one SNP is detected using primers comprising pairs of sequences selected from:
SEQ ID NO:29 and SEQ ID NO:30 for detection of SNP1,
SEQ ID NO:31 and SEQ ID NO:32 for detection of SNP2,
SEQ ID NO:33 and SEQ ID NO:34 for detection of SNP3,
SEQ ID NO:35 and SEQ ID NO:36 for detection of SNP4-5,
SEQ ID NO:37 and SEQ ID NO:38 for detection of SNP6,
SEQ ID NO:39 and SEQ ID NO:40 for detection of SNP7-8,
SEQ ID NO:41 and SEQ ID NO:42 for detection of SNP9,
SEQ ID NO:43 and SEQ ID NO:44 for detection of SNP10-11,
SEQ ID NO:45 and SEQ ID NO:46 for detection of SNP12-13,
SEQ ID NO:47 and SEQ ID NO:48 for detection of SNP14,
SEQ ID NO:49 and SEQ ID NO:50 for detection of SNP15-16,
SEQ ID NO:51 and SEQ ID NO:50 for detection of SNP16,
SEQ ID NO:52 and SEQ ID NO:53 for detection of SNP17,
SEQ ID NO:54 and SEQ ID NO:55 for detection of SNP18,
SEQ ID NO:56 and SEQ ID NO:57 for detection of SNP19,
SEQ ID NO:58 and SEQ ID NO:59 for detection of SNP20,
SEQ ID NO:60 and SEQ ID NO:61 for detection of SNP21,
SEQ ID NO:62 and SEQ ID NO:63 for detection of SNP22,
SEQ ID NO:64 and SEQ ID NO:65 for detection of SNP23-24,
SEQ ID NO:66 and SEQ ID NO:67 for detection of SNP25-26,
SEQ ID NO:68 and SEQ ID NO:69 for detection of SNP27,
SEQ ID NO:70 and SEQ ID NO:71 for detection of SNP28,
SEQ ID NO:72 and SEQ ID NO:73 for detection of SNP29,
SEQ ID NO:74 and SEQ ID NO:75 for detection of SNP30,
SEQ ID NO:76 and SEQ ID NO:77 for detection of SNP31,
SEQ ID NO:78 and SEQ ID NO:79 for detection of SNP32,
SEQ ID NO:80 and SEQ ID NO:81 for detection of SNP33, and
SEQ ID NO:82 and SEQ ID NO:83 for detection of SNP34.

32. An isolated cell of the cultivated Phytoseiulus persimilis (P. persimilis) predatory mite according to claim 28.

33. A Phytoseiulus persimilis rearing mite population, wherein at least 5% of the population are the cultivated P. persimilis mite individuals according to claim 28.

34. (canceled)

35. The Phytoseiulus persimilis rearing mite population according to claim 33, wherein the population is characterized by improved rearing properties on a non-Tetranychid arthropod prey as a food source, preferably on an immobilized non-Tetranychid arthropod prey, such as on an Astigmatid prey, most preferably on an immobilized Astigmatid prey.

36-38. (canceled)

39. A method for controlling a pest in a crop comprising providing the crop with a Phytoseiulus persimilis rearing mite population according to claim 33.

40. A container for releasing mite individuals of a Phytoseiulus persimilis predatory mite species, holding a biological control composition according to claim 1, comprising wherein the device comprises an exit for mobile life stages of the P. persimilis predatory mite species, preferably an exit suitable for providing a sustained release of a number of mobile life stages.

41-43. (canceled)

44. A method for identifying and/or selecting Phytoseiulus persimilis (P. persimilis) predatory mite individuals according to claim 10, characterized by improved rearing properties on a non-Tetranychid arthropod prey as a food source, preferably on an immobilized non-Tetranychid arthropod prey, such as on an Astigmatid prey, most preferably on an immobilized Astigmatid prey, wherein said method comprises detecting in the genome of the P. persimilis predatory mite individuals, the presence of a genomic haplotype comprising a single nucleotide polymorphism (SNP) marker at a position corresponding to at least one of:
SNP1 at position 163 of a sequence having at least 90% sequence similarity to SEQ ID NO:1 or complementary sequence thereof,
SNP2 at position 181 of a sequence having at least 90% sequence similarity to SEQ ID NO:2 or complementary sequence thereof,
SNP3 at position 197 of a sequence having at least 90% sequence similarity to SEQ ID NO:3 or complementary sequence thereof,
SNP4 at position 111 of a sequence having at least 90% sequence similarity to SEQ ID NO:4 or complementary sequence thereof,
SNP5 at position 165 of a sequence having at least 90% sequence similarity to SEQ ID NO:4 or complementary sequence thereof,
SNP6 at position 193 of a sequence having at least 90% sequence similarity to SEQ ID NO:5 or complementary sequence thereof,
SNP7 at position 186 of a sequence having at least 90% sequence similarity to SEQ ID NO:6 or complementary sequence thereof,
SNP8 at position 193 of a sequence having at least 90% sequence similarity to SEQ ID NO:6 or complementary sequence thereof,
SNP9 at position 161 of a sequence having at least 90% sequence similarity to SEQ ID NO:7 or complementary sequence thereof,
SNP10 at position 203 of a sequence having at least 90% sequence similarity to SEQ ID NO:8 or complementary sequence thereof,
SNP11 at position 213 of a sequence having at least 90% sequence similarity to SEQ ID NO:8 or complementary sequence thereof,
SNP12 at position 163 of a sequence having at least 90% sequence similarity to SEQ ID NO:9 or complementary sequence thereof,
SNP13 at position 192 of a sequence having at least 90% sequence similarity to SEQ ID NO:9 or complementary sequence thereof,
SNP14 at position 208 of a sequence having at least 90% sequence similarity to SEQ ID NO:10 or complementary sequence thereof,
SNP15 at position 199 of a sequence having at least 90% sequence similarity to SEQ ID NO:11 or complementary sequence thereof,
SNP16 at position 266 of a sequence having at least 90% sequence similarity to SEQ ID NO:12 or complementary sequence thereof,
SNP17 at position 216 of a sequence having at least 90% sequence similarity to SEQ ID NO:13 or complementary sequence thereof,
SNP18 at position 187 of a sequence having at least 90% sequence similarity to SEQ ID NO:14 or complementary sequence thereof,
SNP19 at position 279 of a sequence having at least 90% sequence similarity to SEQ ID NO:15 or complementary sequence thereof,
SNP20 at position 188 of a sequence having at least 90% sequence similarity to SEQ ID NO:16 or complementary sequence thereof,
SNP21 at position 165 of a sequence having at least 90% sequence similarity to SEQ ID NO:17 or complementary sequence thereof,
SNP22 at position 183 of a sequence having at least 90% sequence similarity to SEQ ID NO:18 or complementary sequence thereof,
SNP23 at position 133 of a sequence having at least 90% sequence similarity to SEQ ID NO:19 or complementary sequence thereof,
SNP24 at position 182 of a sequence having at least 90% sequence similarity to SEQ ID NO:19 or complementary sequence thereof,
SNP25 at position 146 of a sequence having at least 90% sequence similarity to SEQ ID NO:20 or complementary sequence thereof,
SNP26 at position 323 of a sequence having at least 90% sequence similarity to SEQ ID NO:20 or complementary sequence thereof,
SNP27 at position 241 of a sequence having at least 90% sequence similarity to SEQ ID NO:21 or complementary sequence thereof,
SNP28 at position 236 of a sequence having at least 90% sequence similarity to SEQ ID NO:22 or complementary sequence thereof,
SNP29 at position 199 of a sequence having at least 90% sequence similarity to SEQ ID NO:23 or complementary sequence thereof,
SNP30 at position 185 of a sequence having at least 90% sequence similarity to SEQ ID NO:24 or complementary sequence thereof,
SNP31 at position 158 of a sequence having at least 90% sequence similarity to SEQ ID NO:25 or complementary sequence thereof,
SNP32 at position 143 of a sequence having at least 90% sequence similarity to SEQ ID NO:26 or complementary sequence thereof,
SNP33 at position 157 of a sequence having at least 90% sequence similarity to SEQ ID NO:27 or complementary sequence thereof,
SNP34 at position 161 of a sequence having at least 90% sequence similarity to SEQ ID NO:28 or complementary sequence thereof, and any combination thereof.

45. (canceled)

46. The method according to claim 44, wherein said Phytoseiulus predatory individual comprising a polynucleotide genomic sequence selected from SEQ ID NO:1 to SEQ ID NO:28, and any combination thereof, and/or the detection is performed using at least one primer pair selected from:
SEQ ID NO:29 and SEQ ID NO:30 for detection of SNP1,
SEQ ID NO:31 and SEQ ID NO:32 for detection of SNP2,
SEQ ID NO:33 and SEQ ID NO:34 for detection of SNP3,
SEQ ID NO:35 and SEQ ID NO:36 for detection of SNP4-5,
SEQ ID NO:37 and SEQ ID NO:38 for detection of SNP6,
SEQ ID NO:39 and SEQ ID NO:40 for detection of SNP7-8,
SEQ ID NO:41 and SEQ ID NO:42 for detection of SNP9,
SEQ ID NO:43 and SEQ ID NO:44 for detection of SNP10-11,
SEQ ID NO:45 and SEQ ID NO:46 for detection of SNP12-13,
SEQ ID NO:47 and SEQ ID NO:48 for detection of SNP14,
SEQ ID NO:49 and SEQ ID NO:50 for detection of SNP15-16,
SEQ ID NO:51 and SEQ ID NO:50 for detection of SNP16,
SEQ ID NO:52 and SEQ ID NO:53 for detection of SNP17,
SEQ ID NO:54 and SEQ ID NO:55 for detection of SNP18,
SEQ ID NO:56 and SEQ ID NO:57 for detection of SNP19,
SEQ ID NO:58 and SEQ ID NO:59 for detection of SNP20,
SEQ ID NO:60 and SEQ ID NO:61 for detection of SNP21,
SEQ ID NO:62 and SEQ ID NO:63 for detection of SNP22,
SEQ ID NO:64 and SEQ ID NO:65 for detection of SNP23-24,
SEQ ID NO:66 and SEQ ID NO:67 for detection of SNP25-26,
SEQ ID NO:68 and SEQ ID NO:69 for detection of SNP27,
SEQ ID NO:70 and SEQ ID NO:71 for detection of SNP28,
SEQ ID NO:72 and SEQ ID NO:73 for detection of SNP29,
SEQ ID NO:74 and SEQ ID NO:75 for detection of SNP30,
SEQ ID NO:76 and SEQ ID NO:77 for detection of SNP31,
SEQ ID NO:78 and SEQ ID NO:79 for detection of SNP32,
SEQ ID NO:80 and SEQ ID NO:81 for detection of SNP33,
SEQ ID NO:82 and SEQ ID NO:83 for detection of SNP34.

47-61. (canceled)

62. An isolated nucleic acid sequence comprising at least 90% similarity to a polynucleotide sequence selected from (a) at least one sequence selected from SEQ ID NO:1 to SEQ ID NO: 28 and SEQ ID NO:84 to SEQ ID NO:91 and any combination thereof, and/or (b) at least one sequence pair selected from
SEQ ID NO:29 and SEQ ID NO:30,
SEQ ID NO:31 and SEQ ID NO:32,
SEQ ID NO:33 and SEQ ID NO:34,
SEQ ID NO:35 and SEQ ID NO:36
SEQ ID NO:37 and SEQ ID NO:38,
SEQ ID NO:39 and SEQ ID NO:40,
SEQ ID NO:41 and SEQ ID NO:42,
SEQ ID NO:43 and SEQ ID NO:44,
SEQ ID NO:45 and SEQ ID NO:46,
SEQ ID NO:47 and SEQ ID NO:48,
SEQ ID NO:49 and SEQ ID NO:50,
SEQ ID NO:51 and SEQ ID NO:50,
SEQ ID NO:52 and SEQ ID NO:53,
SEQ ID NO:54 and SEQ ID NO:55,
SEQ ID NO:56 and SEQ ID NO:57,
SEQ ID NO:58 and SEQ ID NO:59,
SEQ ID NO:60 and SEQ ID NO:61,
SEQ ID NO:62 and SEQ ID NO:63,
SEQ ID NO:64 and SEQ ID NO:65,
SEQ ID NO:66 and SEQ ID NO:67,
SEQ ID NO:68 and SEQ ID NO:69,
SEQ ID NO:70 and SEQ ID NO:71,
SEQ ID NO:72 and SEQ ID NO:73,
SEQ ID NO:74 and SEQ ID NO:75,
SEQ ID NO:76 and SEQ ID NO:77,
SEQ ID NO:78 and SEQ ID NO:79,
SEQ ID NO:80 and SEQ ID NO:81, and
SEQ ID NO:82 and SEQ ID NO:83,
for use in genetic-characterization of improved rearing properties in predatory mites.

63-70. (canceled)
